(19) United States
(12) Patent Application Publication
(10) Pub. No.: US 2010/0276301 A1
(43) Pub. Date: Nov. 4, 2010

(54) Method and Apparatus for Treating a Liquid

(75) Inventor: Bruce F. Field, Golden Valley, MN (US)

Correspondence Address: WESTMAN CHAMPLIN & KELLY, P.A., SUITE 1400, 900 SECOND AVENUE SOUTH, MINNEAPOLIS, MN, 55402, US

(73) Assignee: TENNANT COMPANY, Minneapolis, MN (US)

(21) Appl. No.: 12/835,441

(22) Filed: Jul. 13, 2010

Related U.S. Application Data
(63) Continuation of application No. 12/639,628, filed on Dec. 16, 2009.
(60) Provisional application No. 61/138,465, filed on Dec. 17, 2008.
(60) Provisional application No. 61/248,557, filed on Oct. 5, 2009.

(51) Int. Cl. B08B 3/04; C02F 1/461; C25B 9/04; A61L 2/03; C25F 1/00

(52) U.S. Cl. 205/701; 204/224 R; 204/232; 204/230.2; 210/282; 210/259; 205/687

(57) ABSTRACT

An apparatus and method are provided. The apparatus includes first and second liquid lines, a container and a dispenser. The first liquid line is configured to receive a liquid. The container is coupled to the first liquid line and is configured to retain media of at least one liquid-activating material. The media of the at least one liquid-activating material is configured to modify an oxidation reduction potential of the received liquid to provide a treated liquid. The second liquid line is coupled to the container and is configured to receive the treated liquid from the container. The dispenser is operably coupled to the second liquid line at a location downstream from the container and is configured to dispense the treated liquid.

### CROSS-REFERENCE TO RELATED APPLICATIONS

The present application is a continuation of, and claims the benefit of U.S. patent application Ser. No. 12/639,628, filed Dec. 16, 2009, which is based on and claims the benefit of U.S. Provisional Patent Application No. 61/138,465, filed Dec. 17, 2008, and U.S. Provisional Patent Application No. 61/248,557, filed Oct. 5, 2009, the contents of which are hereby incorporated by reference in their entireties.

### FIELD OF THE DISCLOSURE

The present disclosure relates to, for example, apparatus and methods for cleaning and/or sanitizing surfaces. In one particular example, the disclosure relates to apparatus and methods that modify an oxidation-reduction potential of a liquid used for cleaning and/or sanitizing.

### BACKGROUND

Electrolysis cells are used in a variety of different applications for changing one or more characteristics of a fluid. For example, electrolysis cells have been used in cleaning/sanitizing applications, medical industries, and semiconductor manufacturing processes. Electrolysis cells have also been used in a variety of other applications and have had different configurations.

For cleaning/sanitizing applications, electrolysis cells are used to create anolyte electrochemically activated (EA) liquid and catholyte EA liquid. Anolyte EA liquids have known sanitizing properties, and catholyte EA liquids have known cleaning properties. Examples of cleaning and/or sanitizing systems are disclosed in Field et al. U.S. Publication No. 2007/0186368 A1, published Aug. 16, 2007.

However, the sanitizing capabilities of anolyte EA liquids can be limited in some applications. An aspect, among others, of the present application is directed to improved methods, systems and/or apparatus for enhancing sanitizing properties of a liquid.

### SUMMARY

An aspect of the disclosure relates to an apparatus, which includes a mobile body configured to travel over a surface, a first liquid line configured to receive a liquid, and a container coupled to the first liquid line. The container retains media of at least one liquid-activating material, the media of the at least one liquid-activating material being configured to modify an oxidation reduction potential of the received liquid to provide a treated liquid. A second liquid line is coupled to the container and is configured to receive the treated liquid from the container. A dispenser is operably coupled to the second liquid line at a location downstream from the container and is configured to dispense the treated liquid.

Another aspect of the disclosure relates to a cartridge for use with a surface cleaning apparatus. The cartridge includes a housing that defines an interior chamber, a first housing interface configured to provide access to the interior chamber and to be removably engaged with an upstream liquid line of the surface cleaning apparatus, and a second housing interface configured to provide access to the interior chamber and be removably engaged with a downstream liquid line of the surface cleaning apparatus. The container further includes media of at least one liquid-activating material retained in the interior chamber of the housing. The media of the at least one liquid-activating material is configured to modify an oxidation reduction potential of liquid flowing through the interior chamber of the cartridge.

Another aspect of the disclosure relates to a method for operating a surface cleaning apparatus. The method includes: moving the surface cleaning apparatus relative to a surface; introducing a liquid from a first liquid line of the surface cleaning apparatus to a container carried by the surface cleaning apparatus, the container retaining media of at least one liquid-activating material; treating the introduced liquid with the media to modify an oxidation reduction potential of the introduced liquid to provide a treated liquid; and dispensing the treated liquid from a dispenser of the surface cleaning apparatus located downstream from the container.

An additional aspect of the disclosure relates to an apparatus, which includes first and second liquid lines, a container and a dispenser. The first liquid line is configured to receive a liquid. The container is coupled to the first liquid line and is configured to retain media of at least one liquid-activating material. The media of the at least one liquid-activating material is configured to modify an oxidation reduction potential of the received liquid to provide a treated liquid. The second liquid line is coupled to the container and is configured to receive the treated liquid from the container. The dispenser is operably coupled to the second liquid line at a location downstream from the container and is configured to dispense the treated liquid.

### BRIEF DESCRIPTION OF THE DRAWINGS

FIG. 1 is a simplified, schematic diagram of an example of a hand-held spray bottle according to an exemplary aspect of the present disclosure.

FIG. 2 illustrates an example of an electrolysis cell having an ion-selective membrane.

FIG. 3 illustrates an electrolysis cell having no ion-selective membrane according to a further example of the disclosure.

FIGS. 4A-4D are diagrams illustrating an example of a dirt cleaning mechanism performed by a liquid that is electrochemically-activated according to an aspect of the disclosure.

FIG. 5 illustrates an example of an electrolysis cell having a tubular shape according to an illustrative example.

FIG. 6 is an exploded, perspective view of an electroporation electrode according to an illustrative example of the disclosure.

FIG. 7A is a diagram illustrating an example of conductive paths formed between a spray head and a surface by an electrically charged output spray.

FIG. 7B is a diagram illustrating an example of an electroporation mechanism, whereby a cell suspended in a medium is subjected to an electric field.

FIG. 7C is a diagram illustrating an example of a cell membrane having pores expanded by electroporation.

FIG. 8 is a diagram illustrating an example of a spray bottle spraying an electrically charged liquid to a surface.

FIG. 9 is a diagram illustrating an example of a surface being sprayed and wetted with an electrically charged liquid.

FIG. 10A is a perspective view of a hand-held spray bottle according to an embodiment of the disclosure.

FIG. 10B is a perspective view of an exposed left-half of the hand-held spray bottle according to an embodiment of the disclosure.

FIG. 10C is a side view of an exposed spray head of the hand-held spray bottle according to an embodiment of the disclosure.

FIG. 11 is a waveform diagram illustrating an example of the voltage pattern applied to the anode and cathode of an electrolysis cell in the spray bottle according to an exemplary aspect of the present disclosure.

FIG. 12 is a block diagram of an example of a control circuit for controlling the electrolysis cell on the spray bottle according to an exemplary aspect of the disclosure.

FIG. 13A is an example of a waveform diagram illustrating the voltage pattern applied to an electroporation electrode in the spray bottle according to an exemplary aspect of the present disclosure.

FIG. 13B is an example of a waveform diagram illustrating a frequency pattern applied to an electroporation electrode in the spray bottle according to an exemplary aspect of the present disclosure.

FIG. 13C is an example of a waveform diagram illustrating a frequency pattern applied to an electroporation electrode in the spray bottle according to an exemplary aspect of the present disclosure.

FIG. 14 is a block diagram of an example of a control circuit for controlling the electroporation electrode on the spray bottle according to an exemplary aspect of the disclosure.

FIG. 15 is a perspective view of an example of a mobile floor cleaning machine according to another embodiment of the disclosure.

FIG. 16 is a perspective view of an example of an all-surface cleaner according to another embodiment of the disclosure.

FIG. 17 is a diagram illustrating an example of a flat mop embodiment, which includes at least one electrolysis cell and/or at least one electroporation electrode, such as those described in the present disclosure.

FIG. 18 is a diagram illustrating an example device, which can be stationary or movable relative to a surface.

FIG. 19 is a block diagram, which illustrates a system according to an example embodiment of the disclosure, which can be incorporated into any of the embodiments disclosed herein, for example.

FIGS. 20A and 20B are graphs, which plot examples of the potential field and electric field, respectively, as a function of distance from the nozzle for the embodiment shown in FIGS. 5-6 and 10-14, for example.

FIG. 21 is a diagram illustrating a system according to an example embodiment of the disclosure in which a suspension additive is added to a liquid dispensed from an apparatus to enhance suspension properties of the dispensed liquid.

FIG. 22 is a schematic illustration of a spray bottle configured to retain one or more liquid-activating materials for altering the oxidation-reduction potential (ORP) of liquids retained and dispensed by the spray bottle, for example.

FIG. 23 is a schematic illustration of a cartridge containing a liquid-activating material, which may be installed in a fluid line of a flow-through system, for example.

### DETAILED DESCRIPTION OF ILLUSTRATIVE EMBODIMENTS

The following is provided as additional description of examples of one or more aspects of the present disclosure. The below detailed description and above-referenced Figures should not to be read as limiting or narrowing the scope of the invention as will be claimed in issued claims. It will be appreciated that other embodiments of the invention covered by one or more of the claims may have structure and function which are different in one or more aspects from the figures and examples discussed herein, and may embody different structures, methods and/or combinations thereof of making or using the invention as claimed in the claims, for example.

Also, the following description is divided into sections with one or more section headings. These sections and headings are provided for ease of reading only and, for example, do not limit one or more aspects of the disclosure discussed in a particular section and/or section heading with respect to a particular example and/or embodiment from being combined with, applied to, and/or utilized in another particular example, and/or embodiment which is described in another section and/or section heading. Elements, features and other aspects of one or more examples may be combined and/or interchangeable with elements, features and other aspects of one or more other examples described herein.

An aspect of the present disclosure for example relates to enhancing sanitization properties of an output fluid (including a liquid stream and/or a gas/liquid mixture, water vapor, gaseous liquid, mist, spray or aerosol mixture for example) that is dispensed from an apparatus. In one example, the disclosure relates to enhancing sanitization properties of an output liquid (including a liquid stream and/or a gas/liquid mixture, gaseous liquid, mist, spray or aerosol mixture for example). An exemplary basis for sanitization in one or more examples of the present disclosure includes applying an electric field, such as an alternating electric field, to cells of a microorganism on a surface being treated, wherein the electric field meets or surpasses a threshold such that the cells become permanently damaged by a process known as irreversible electroporation, for example. If the electric field threshold is reached or surpassed, electroporation will compromise the viability of the cells, resulting in irreversible electroporation.

In one or more examples, the microorganisms are suspended from the surface by liquid dispensed from the apparatus and through which an electric field is applied. Such suspension can be enhanced, for example by altering the oxidation-reduction potential of the liquid to exceed about +/−50 milivolts, for example. Suspension of the microorganisms may enhance application of the electric field to cells of the microorganism.

In a particular example, an aspect of the present disclosure relates to a method and apparatus for enhancing sanitization properties of electrolyzed liquids produced by an electrolysis cell carried by a stationary or movable apparatus, such as a hand-held spray bottle or device, a mobile floor cleaner, a hand sanitizing station or device, a food sanitizer, fabric or dish washing machine, and/or other apparatus for generating or applying a liquid and/or gas/liquid mixture to a surface or volume of space. The electrolysis cell can, for example, increase the ORP of a liquid to aid in suspension of the microorganisms through the action of charged nanobubbles, for example. Other mechanisms can also be used to alter a liquid's ORP and/or enhance suspension of particles and microorganisms from a surface.

Embodiments of the present disclosure can be used in a variety of different applications and housed in a variety of different types of apparatus, including but not limited to apparatus that are hand-held, mobile, immobile, wall-mounted, motorized or non-motorized, wheeled or non-wheeled, etc. In the following example, an electrolysis cell and an electroporation electrode are incorporated in a hand-held spray bottle. It will be appreciated that one or more of the various aspects of one or more of the examples discussed in the present disclosure may be combined with and/or substituted for other aspects in alternate embodiments as appropriate. The headings set out herein are utilized for convenience and are not intended, for example, to limit aspects of an embodiment discussed under that to that or a particular embodiment or example. Also, for example, although the term “electroporation electrode” is used in the description to refer to an electrode, this term is used for convenience only and is not intended to limit its operation or effect on microorganisms to a process of electroporation.

In the one or more examples of the present disclosure, instead of using traditional electrical probes for example to deliver an applied electric field, an apparatus may be configured to deliver such an applied electric field through a charged output liquid.

### 1. Hand-Held Spray Device Example

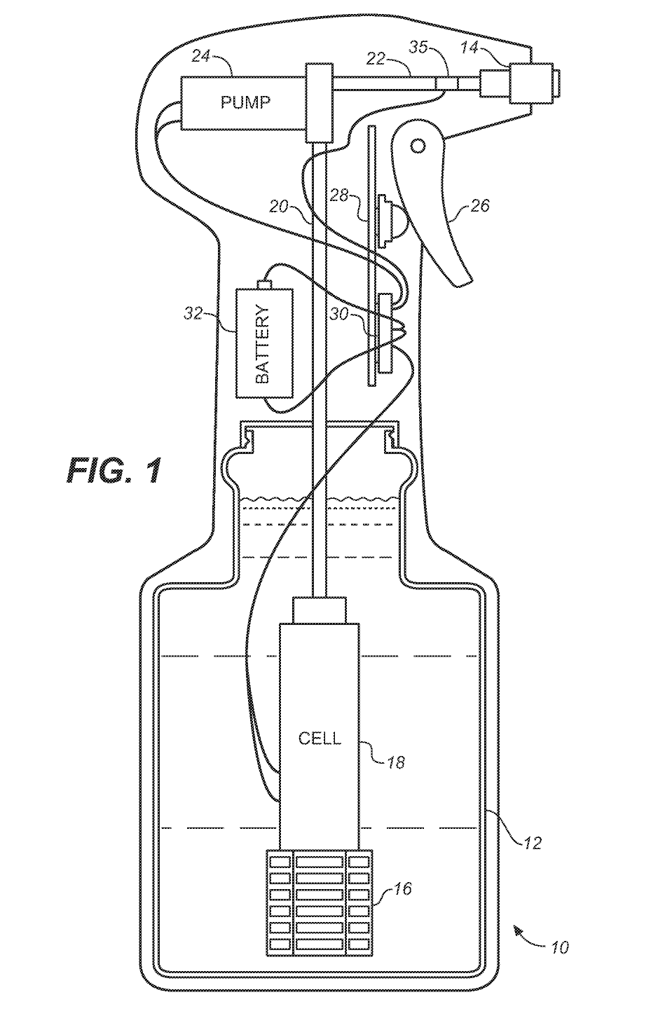

FIG. 1 is a simplified, schematic diagram of an example of a hand-held spray device, here in the form of a hand-held spray bottle 10 according to an exemplary aspect of the present disclosure. In another example, the spray device may form part of a larger device or system. In the example shown in FIG. 1, spray bottle 10 includes a reservoir 12 for containing a liquid to be treated and then dispensed through a nozzle 14. In an example, the liquid to be treated includes an aqueous composition, such as regular tap water.

Spray bottle 10 further includes an inlet filter 16, one or more electrolysis cells 18, tubes 20 and 22, pump 24, actuator 26, switch 28, circuit board and control electronics 30 and batteries 32. Although not shown in FIG. 1, tubes 20 and 22 may be housed within a neck and barrel, respectively of bottle 10, for example. A cap seals reservoir 12 around the neck of bottle 10. Batteries 32 can include disposable batteries and/or rechargeable batteries, for example, or other appropriate portable or corded electrical source in addition to or in place of batteries, to provide electrical power to electrolysis cell 18 and pump 24 when energized by circuit board and control electronics 30.

In the example shown in FIG. 1, actuator 26 is a trigger-style actuator, which actuates momentary switch 28 between open and closed states. For example, when the user squeezes the hand trigger, the trigger actuates the switch from the open state to the closed state. When the user releases the hand trigger, the trigger actuates the switch into the open state. However, actuator 26 can have other styles or structure in alternative embodiments and can be eliminated in further embodiments. In embodiments that lack a separate actuator, switch 28 for example can be actuated directly by a user. When switch 28 is in the open, non-conducting state, control electronics 30 de-energizes electrolysis cell 18 and pump 24. When switch 28 is in the closed, conducting state, control electronics 30 energizes electrolysis cell 18 and pump 24. Pump 24 draws liquid from reservoir 12 through filter 16, electrolysis cell 18, and tube 20 and forces the liquid out tube 22 and nozzle 14. Depending on the sprayer, nozzle 14 may or may not be adjustable, so as to select between squirting a stream, aerosolizing a mist, or dispensing a spray, for example.

Switch 28, itself, can have any suitable actuator type, such as a push-button switch as shown in FIG. 1, a toggle, a rocker, any mechanical linkage, and/or any sensor to sense input, including for example capacitive, resistive plastic, thermal, inductive, mechanical, non-mechanical, electro-mechanical, or other sensor, etc. Switch 28 can have any suitable contact arrangement, such as momentary, single-pole single throw, etc.

In an alternative embodiment, pump 24 is replaced with a mechanical pump, such as a hand-triggered positive displacement pump, wherein actuator trigger 26 acts directly on the pump by mechanical action. In this embodiment, switch 28 could be separately actuated from the pump 24, such as a power switch, to energize electrolysis cell 18. In a further embodiment, batteries 32 are eliminated and power is delivered via another portable source, e.g., a rotating dynamo, shaker or solar source etc., or delivered to spray bottle 10 from an external source, such as through a power cord, plug, and/or contact terminals. For example, in an alternate embodiment a user may actuate an internal dynamo while squeezing the trigger in order to generate electrical power. The spray bottle can comprise any suitable power source, such as a portable power source carried by the bottle or terminals carried by the bottle for connecting to an external power source.

The arrangement shown in FIG. 1 is provided merely as a non-limiting example. Spray bottle 10 can have any other structural and/or functional arrangement. For example, pump 24 can be located downstream of cell 18, as shown in FIG. 1, or upstream of cell 18 with respect to the direction of fluid flow from reservoir 12 to nozzle 14. Spray bottle 10 may be any other appropriate hand-held device for example, and need not be in the shape of a bottle, or spray bottle. Other form factors or ergonomic shapes for example may be utilized in other embodiments. For example, the spray device may have the form of a wand, which may or may not be connected to a cleaning device, such as a mop bucket, a motorized or non-motorized all-purpose cleaner, a mobile cleaning device with or without a separate cleaning head, a vehicle, etc.

As described in more detail below, the spray bottle contains a liquid to be sprayed on a surface or into a volume of space to be cleaned and/or sanitized. In one non-limiting example, electrolysis cell 18 converts the liquid to an anolyte EA liquid and a catholyte EA liquid prior to being dispensed from the nozzle 14 as an output spray (or stream, for example). The anolyte and catholyte EA liquids can be dispensed as a combined mixture or as separate spray outputs, such as through separate tubes and/or nozzles. In the embodiment shown in FIG. 1, the anolyte and catholyte EA liquids are dispensed as a combined mixture. With a small and intermittent output flow rate provided the spray bottle, electrolysis cell 18 can have a small package and be powered by batteries carried by the package or spray bottle, for example.

Spray bottle 10 can further include a separate electrical conductor, lead, or other electrical and/or electromagnetic component, for example an electrode, e.g., high voltage electrode 35, which is positioned in, or in appropriate relation to, the liquid or liquid path to impart, induce or otherwise cause an electrical potential in the liquid output spray relative to Earth ground, for example. If a liquid forming a liquid output spray, for instance, already carries a charge, such an electrical potential can be a separate or additional electrical potential in the liquid output spray, for example. In the example shown in FIG. 1, electrode 35 is positioned along tube 22 and is configured to make electrical contact with the liquid flowing through the tube. However, electrode 35 can be located at any position along the liquid flow path from reservoir 12 to nozzle 14 (or even external to spray bottle 10) for example. Control circuit 30 energizes electrode 35 when trigger 26 actuates switch 28 into the closed state, and de-energizes electrode 35 when trigger 26 actuates switch 28 into the open state. It will be appreciated that other energizing, de-energizing states or patterns could be used in other embodiments, such as de-energizing electrode 35 even during part of the time trigger 26 is operated and/or liquid is being dispensed, for example. In this example, electrode 35 has no corresponding return electrode of opposite polarity. Further, in other embodiments more than one electrical conductor, lead, or other electrical component or combination thereof could be utilized to impart, induce or otherwise cause an electrical potential.

Electrical potential created and/or supplemented by electrode 35 is applied to microorganisms on the surface being cleaned through liquid dispensed and, if the charge delivery is of a sufficient magnitude, such a charge can cause irreversible damage, destruction to or otherwise eliminate microorganisms through a mechanism such as electroporation and/or elecrohydraulic shock, as discussed in examples in more detail below. This enhances sanitization properties of the liquid output spray during use.

### 2. Electrolysis Cells Example

An electrolysis cell includes any fluid treatment cell that is adapted to apply an electric field across the fluid between at least one anode electrode and at least one cathode electrode. An electrolysis cell can have any suitable number of electrodes, any suitable number of chambers for containing the fluid, and any suitable number of fluid inputs and fluid outputs. The cell can be adapted to treat any fluid (such as a liquid or gas-liquid combination). The cell can include one or more ion-selective membranes between the anode and cathode or can be configured without any ion selective membranes. An electrolysis cell having an ion-selective membrane is referred to in this example as a “functional generator”. This term is not intended to limiting; it will be appreciated that other appropriate device and/or structure may qualify as a functional generator.

Electrolysis cells can be used in a variety of different applications and can have a variety of different structures, such as but not limited to a spray bottle as discussed with reference to FIG. 1, and/or the structures disclosed in Field et al. U.S. Patent Publication No. 2007/0186368, published Aug. 16, 2007 and incorporated herein in its entirety. Thus, although various elements and processes relating to electrolysis are described herein relative to the context of a spray bottle, these elements and processes can be applied to, and incorporated in, other, non-spray bottle applications.

### 2.1 Electrolysis Cell Having a Membrane Example

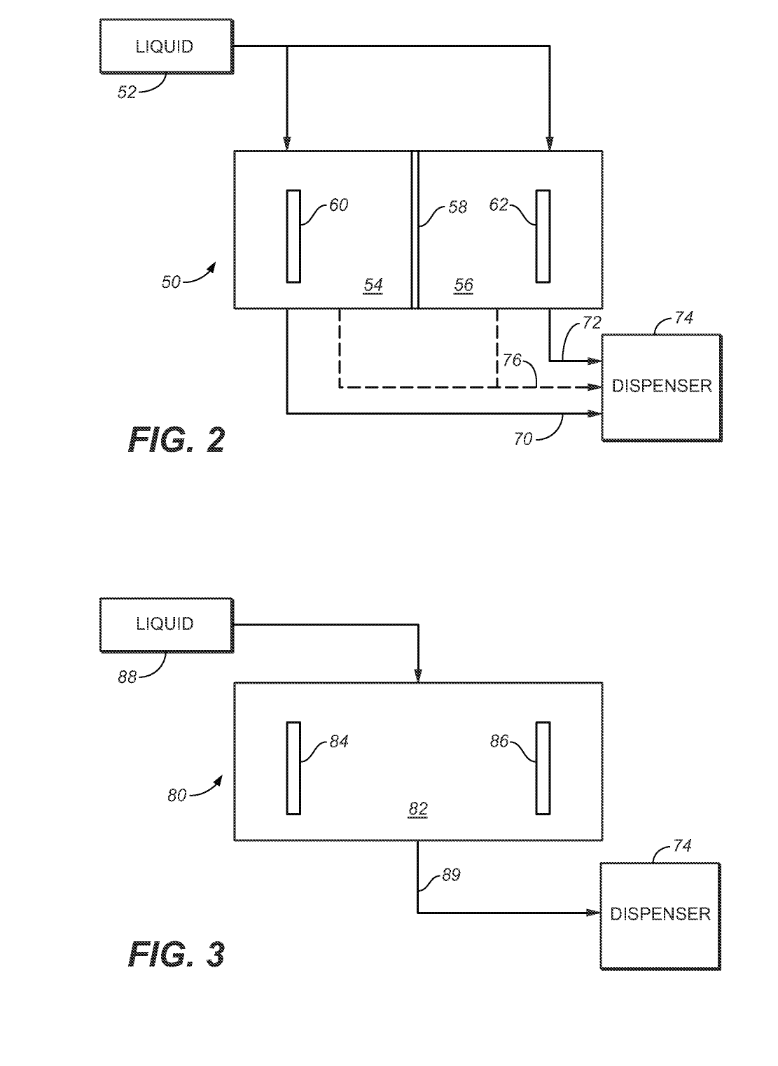

FIG. 2 is a schematic diagram illustrating an example of an electrolysis cell 50 that can be used in the spray bottle shown in FIG. 1, for example. Electrolysis cell 50 receives liquid to be treated from a liquid source 52. Liquid source 52 can include a tank or other solution reservoir, such as reservoir 12 in FIG. 1, or can include a fitting or other inlet for receiving a liquid from an external source.

Cell 50 has one or more anode chambers 54 and one or more cathode chambers 56 (known e.g. as reaction chambers), which are separated by an ion exchange membrane 58, such as a cation (e.g., a proton exchange membrane) or anion exchange membrane. One or more anode electrodes 60 and cathode electrodes 62 (one of each electrode shown) are disposed in each anode chamber 54 and each cathode chamber 56, respectively. The anode and cathode electrodes 60, 62 can be made from any suitable material, for example stainless steel, a conductive polymer, titanium and/or titanium coated with a precious metal, such as platinum, or any other suitable electrode material. In one example, at least one of the anode and cathode is at least partially or wholly made from a conductive polymer. The electrodes and respective chambers can have any suitable shape and construction. For example, the electrodes can be flat plates, coaxial plates, rods, or a combination thereof. Each electrode can have, for example, a solid construction or can have one or more apertures. In one example, each electrode is formed as a mesh. In addition, multiple cells 50 can be coupled in series or in parallel with one another, for example. The electrodes 60, 62 are electrically connected to opposite terminals of a conventional power supply (not shown).

Ion exchange membrane 58 is located between electrodes 60 and 62. The ion exchange membrane 58 can include a cation exchange membrane (e.g., a proton exchange membrane) or an anion exchange membrane. Suitable cation exchange membranes for membrane 38 include partially and fully fluorinated ionomers, polyaromatic ionomers, and combinations thereof. Examples of suitable commercially available ionomers for membrane 38 include sulfonated tetrafluorethylene copolymers available under the trademark “NAFION” from E.I. du Pont de Nemours and Company, Wilmington, Del.; perfluorinated carboxylic acid ionomers available under the trademark “FLEMION” from Asahi Glass Co., Ltd., Japan; perfluorinated sulfonic acid ionomers available under the trademark “ACIPLEX” Aciplex from Asahi Chemical Industries Co. Ltd., Japan; and combinations thereof. Other examples of suitable membranes include, for example, those available from Membranes International Inc. of Glen Rock, N.J., such as the CMI-7000S cation exchange membrane and the AMI-7001S anion exchange membrane. However, any ion exchange membrane can be used in other examples.

The power supply can provide a constant DC output voltage, a pulsed or otherwise modulated DC output voltage, and/or a pulsed or otherwise modulated AC output voltage to the anode and cathode electrodes, for example. The power supply can have any suitable output voltage level, current level, duty cycle or waveform, etc.

For example in one embodiment, the power supply applies the voltage supplied to the plates at a relative steady state. The power supply (and/or control electronics) includes a DC/DC converter that uses a pulse-width modulation (PWM) control scheme to control voltage and current output. Other types of power supplies can also be used, which can be pulsed or not pulsed and at other voltage and power ranges. The parameters may vary depending on a specific application and/or embodiment.

During operation, feed water (or other liquid to be treated) is supplied from source 52 to both anode chamber 54 and cathode chamber 56. In the case of a cation exchange membrane, upon application of a DC voltage potential across anode 60 and cathode 62, such as a voltage in a range of about 5 Volts (V) to about 28V, or for example about 5V to about 38V, cations originally present in the anode chamber 54 move across the ion-exchange membrane 58 towards cathode 62 while anions in anode chamber 54 move towards anode 60. However, anions present in cathode chamber 56 are not able to pass through the cation-exchange membrane, and therefore remain confined within cathode chamber 56.

As a result, cell 50 can electrochemically activate the feed water by at least partially utilizing electrolysis and produces electrochemically-activated water in the form of an acidic anolyte composition 70 and a basic catholyte composition 72. In one example, the anolyte composition 70 has an oxidation-reduction potential (ORP) of at least about +50 mV (e.g., in a range of +50 mV to +1200 mV), and the catholyte composition 72 has an ORP of at least about −50 mV (e.g., in a range of −50 mV to −1000 mV).

If desired, the anolyte and catholyte can be generated in different ratios to one another through modifications to the structure of the electrolysis cell, for example. For example, the cell can be configured to produce a greater volume of catholyte than anolyte if the primary function of the EA water is cleaning. Alternatively, for example, the cell can be configured to produce a greater volume of anolyte than catholyte if the primary function of the EA water is sanitizing. Also, the concentrations of reactive species in each can be varied.

For example, the cell can have a 3:2 ratio of cathode plates to anode plates for producing a greater volume of catholyte than anolyte. Each cathode plate is separated from a respective anode plate by a respective ion exchange membrane. Thus, in this embodiment there are three cathode chambers for two anode chambers. This configuration produces roughly 60% catholyte to 40% anolyte. Other ratios can also be used.

Also, the duty cycle of the applied voltage and/other electrical characteristics can be modified to modify the relative amounts of catholyte and anolyte produced by the cell.

### 2.2. Electrolysis Cell with No Ion-Selective Membrane Example

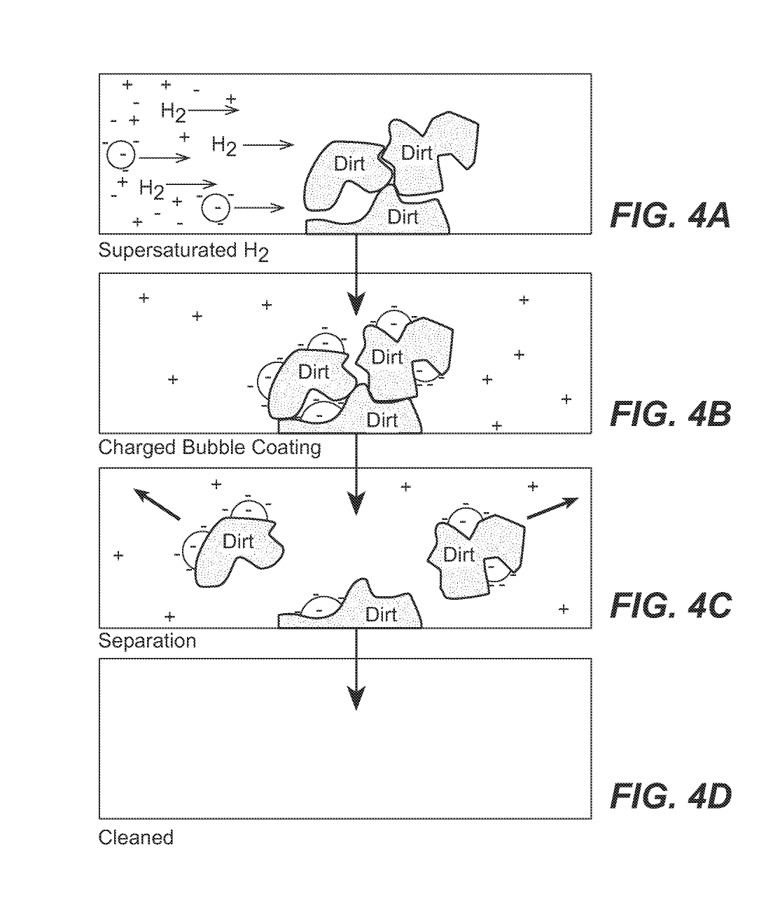

FIG. 3 illustrates an electrolysis cell 80 having no ion-selective membrane according to a further example of the disclosure. Cell 80 includes a reaction chamber 82, an anode 84 and a cathode 86. Chamber 82 can be defined by the walls of cell 80, by the walls of a container or conduit in which electrodes 84 and 86 are placed, or by the electrodes themselves, for example. Anode 84 and cathode 86 may be made from any suitable material or a combination of materials, for example stainless steel, a conductive polymer, titanium and/or titanium coated with a precious metal, such as platinum. Anode 84 and cathode 86 are connected to a conventional electrical power supply, such as batteries 32 shown in FIG. 1. In one embodiment, electrolytic cell 80 includes its own container that defines chamber 82 and is located in the flow path of the liquid to be treated, such as within the flow path of a hand-held spray bottle or mobile floor cleaning apparatus.

During operation, liquid for example is supplied by a source 88 and introduced into reaction chamber 82 of electrolysis cell 80. In the embodiment shown in FIG. 3, electrolysis cell 80 does not include an ion exchange membrane that separates reaction products at anode 84 from reaction products at cathode 86. In the example in which tap water is used as the liquid to be treated for use in cleaning, after introducing the water into chamber 82 and applying a voltage potential between anode 84 and cathode 86, water molecules in contact with or near anode 84 are electrochemically oxidized to oxygen (O₂) and hydrogen ions (H⁺) while water molecules in contact or near cathode 86 are electrochemically reduced to hydrogen gas (H₂) and hydroxyl ions (OH⁻). Other reactions can also occur and the particular reactions depend on the components of the liquid. The reaction products from both electrodes are able to mix and form an oxygenated fluid 89 (for example) since there is no physical barrier, for example, separating the reaction products from each other. Alternatively, for example, anode 84 can be separated from cathode 84 by using a dielectric barrier such as a non-permeable or other membrane (not shown) disposed between the anode and cathode.

### 2.3. Dispenser Example

The anolyte and catholyte EA liquid outputs from FIG. 2 or the oxygenated fluid 89 in FIG. 3 can be coupled to a dispenser 74, which can include any type of dispenser or dispensers, including for example an outlet, fitting, spigot, spray head, a cleaning/sanitizing tool or head, or combination thereof, etc. In the example shown in FIG. 1, dispenser 74 includes spray nozzle 14. There can be a dispenser for each output 70 and 72 in FIG. 2 or a combined dispenser for both outputs.

In one example, the anolyte and catholyte outputs in FIG. 2 are blended into a common output stream 76, which is supplied to dispenser 74. As described in Field et al. U.S. Patent Publication No. 2007/0186368, it has been found that the anolyte and catholyte can be blended together within the distribution system of a cleaning apparatus and/or on the surface or item being cleaned while at least temporarily retaining beneficial cleaning and/or sanitizing properties. Although the anolyte and catholyte are blended, in this example they are initially not in equilibrium and therefore can temporarily retain their enhanced cleaning and/or sanitizing properties.

For example, in one embodiment, the catholyte EA water and the anolyte EA water maintain their distinct electrochemically activated properties for at least 30 seconds, for example, even though the two liquids are blended together. During this time, the distinct electrochemically activated properties of the two types of liquids do not neutralize immediately. This allows the advantageous properties of each liquid in this example to be utilized during a common cleaning operation. After a relatively short period of time, the blended anolyte and catholyte EA liquid on the surface being cleaned may quickly neutralize substantially to the original pH and ORP of the source liquid (e.g., those of normal tap water). In one example, the blended anolyte and catholyte EA liquid neutralize substantially to a pH between pH6 and pH8 and an ORP between ±50 mV within a time window of less than 1 minute or other combinations from the time the anolyte and catholyte EA outputs are produced by the electrolysis cell. Other appropriate pH ranges may result. Thereafter, the recovered liquid can be disposed in any suitable manner.

In other embodiments, the blended anolyte and catholyte EA liquid can maintain e.g. pHs outside of the range between pH6 and pH8 and ORPs outside the range of ±50 mV for a time greater than 30 seconds, and/or can neutralize after a time range that is outside of 1 minute, depending on an embodiment and the properties of the liquid.

### 3. Dirt, and Cleaning with Electrolyzed Water Example

The following discussion as with the other example discussions herein is provided as an example only and not intended to limit the present disclosure, operation of examples described herein and/or the scope of any issued claims appended hereto.

### 3.1 Example of Basic Concepts

Dirt consists of mixtures of dried-on previously-soluble matter, oily material and/or insoluble particles, for example. Generally dirt has a greater affinity for more dirt than it has for water.

To remove dirt, the affinity between dirt particles and other dirt particles, and between the dirt particles and the surface being cleaned, should be reduced and the affinity of dirt particles for water should be increased.

Usually, soaps and detergents are used on oily dirt to form micelles, and polyanions are used to suspend dirt particles. In one exemplary embodiment of the disclosure, neither of these are present in the electrolyzed water dispensed from nozzle 14.

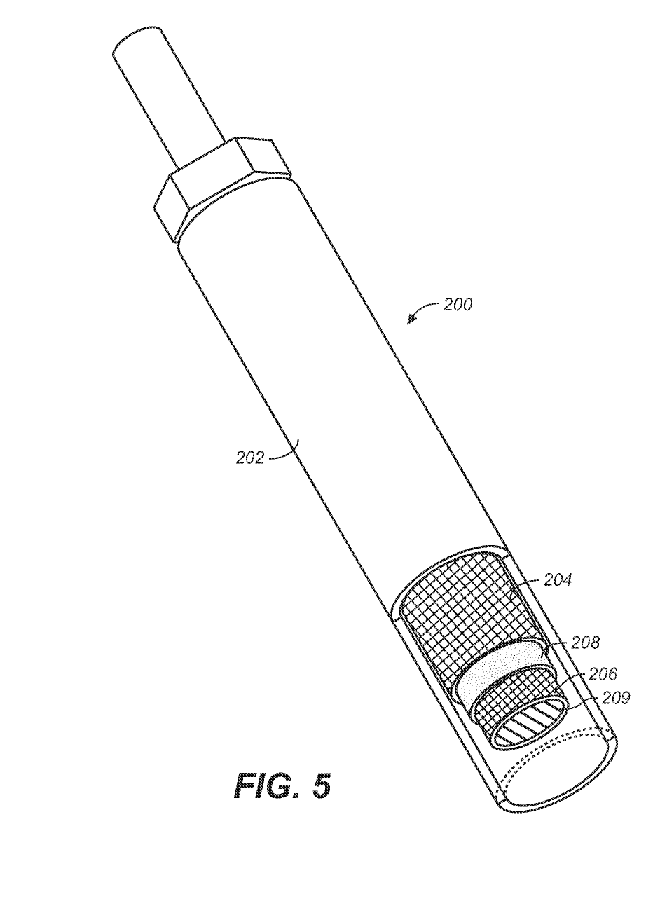

However during the electrolysis process, some nanobubbles are created at the electrode surfaces and then slowly dissipate within the anolyte and catholyte EA liquids produced by the electrolysis cell, as shown in FIG. 4A. Other nanobubbles are created at the dirt surface from the supersaturated EA water solution that is dispensed from the spray bottle. These nanobubbles can exist for significant periods of time both in the aqueous solution and at submerged solid/liquid surfaces.

The nanobubbles tend to form and stick to hydrophobic surfaces, such as those that are found on typical dirt particles, as shown in FIG. 4B. This process is energetically favored as the attachment of the gas bubbles releases water molecules from the high energy water/hydrophobic surface interface with a favorable negative free energy change.

Also, as the bubbles contact the surface, the bubbles spread out and flatten, which reduces the bubbles' curvatures; giving additional favorable free energy release.

Further, the presence of nanobubbles on the surface of dirt particles increases the pick-up of the particle by larger micron-plus sized gas bubbles, possibly introduced by mechanical cleaning/wiping action and/or the prior electrolytic sparging process, as shown in FIG. 4C. The presence of surface nanobubbles also reduces the size of the dirt particle that can be picked up by this action.

Such pick-up helps float away the dirt particles from the surfaces being cleaned and prevents re-deposition, as shown in FIG. 4D.

A further property of nanobubbles is their vast gas/liquid surface area for their volume. Water molecules at this interface are held by fewer hydrogen bonds, as recognized by water's high surface tension. Due to this reduction in hydrogen bonding to other water molecules, the interface water is more reactive than ‘normal’ water and will hydrogen bond to other molecules more rapidly, showing faster hydration.

Due at least in part to these illustrative (example) properties, the combined anolyte and catholyte EA liquid in certain embodiments that is created and dispensed from the spray bottle shown in FIG. 1 has enhanced cleaning properties as compared to non-electrolyzed water.

### 3.2 Example Reactions

With respect to the electrolysis cell 50 shown in FIG. 2, water molecules in contact with anode 60 are electrochemically oxidized to oxygen (O₂) and hydrogen ions (H⁺) in the anode chamber 54 while water molecules in contact with the cathode 62 are electrochemically reduced to hydrogen gas (H₂) and hydroxyl ions (OH⁻) in the cathode chamber 56. The hydrogen ions in the anode chamber 54 are allowed to pass through the cation-exchange membrane 58 into the cathode chamber 56 where the hydrogen ions are reduced to hydrogen gas while the oxygen gas in the anode chamber 54 oxygenates the feed water to form the anolyte 70. Furthermore, since regular tap water typically includes sodium chloride and/or other chlorides, the anode 60 oxidizes the chlorides present to form chlorine gas. As a result, a substantial amount of chlorine is produced and the pH of the anolyte composition 70 becomes increasingly acidic over time.

As noted, water molecules in contact with the cathode 62 are electrochemically reduced to hydrogen gas and hydroxyl ions (OH⁻) while cations in the anode chamber 54 pass through the cation-exchange membrane 58 into the cathode chamber 56 when the voltage potential is applied. These cations are available to ionically associate with the hydroxyl ions produced at the cathode 62, while hydrogen gas bubbles form in the liquid. A substantial amount of hydroxyl ions accumulates over time in the cathode chamber 56 and reacts with cations to form basic hydroxides. In addition, the hydroxides remain confined to the cathode chamber 56 since the cation-exchange membrane does not allow the negatively charged hydroxyl ions pass through the cation-exchange membrane. Consequently, a substantial amount of hydroxides is produced in the cathode chamber 56, and the pH of the catholyte composition 72 becomes increasingly alkaline over time.

The electrolysis process in the functional generator 50 allows concentration of reactive species and the formation of metastable ions and radicals in the anode chamber 54 and cathode chamber 56.

The electrochemical activation process typically occurs by either e.g. electron withdrawal (at anode 60) or electron introduction (at cathode 62), which leads to alteration of physiochemical (including structural, energetic and catalytic) properties of the feed water. It is believed that the feed water (anolyte or catholyte) gets activated in the immediate proximity of the electrode surface where the electric field intensity can reach a very high level. This area can be referred to as an electric double layer (EDL).

While the electrochemical activation process continues, the water dipoles generally align with the field, and a proportion of the hydrogen bonds of the water molecules consequentially break. Furthermore, singly-linked hydrogen atoms bind to the metal atoms (e.g., platinum atoms) at cathode electrode 62, and single-linked oxygen atoms bind to the metal atoms (e.g., platinum atoms) at the anode electrode 60. These bound atoms diffuse around in two dimensions on the surfaces of the respective electrodes until they take part in further reactions. Other atoms and polyatomic groups may also bind similarly to the surfaces of anode electrode 60 and cathode electrode 62, and may also subsequently undergo reactions. Molecules such as oxygen (O₂) and hydrogen (H₂) produced at the surfaces may enter small cavities in the liquid phase of the water (e.g., bubbles) as gases and/or may become solvated by the liquid phase of the water. These gas-phase bubbles are thereby dispersed or otherwise suspended throughout the liquid phase of the feed water.

The sizes of the gas-phase bubbles may vary depending on a variety of factors, such as the pressure applied to the feed water, the composition of the salts and other compounds in the feed water, and the extent of the electrochemical activation. Accordingly, the gas-phase bubbles may have a variety of different sizes, including, but not limited to macrobubbles, microbubbles, nanobubbles, and/or mixtures thereof. In embodiments including macrobubbles, examples of suitable average bubble diameters for the generated bubbles include diameters ranging from about 500 micrometers to about one millimeter. In embodiments including microbubbles, examples of suitable average bubble diameters for the generated bubbles include diameters ranging from about one micrometer to less than about 500 micrometers. In embodiments including nanobubbles, examples of suitable average bubble diameters for the generated bubbles include diameters less than about one micrometer, with particularly suitable average bubble diameters including diameters less than about 500 nanometers, and with even more particularly suitable average bubble diameters including diameters less than about 100 nanometers.

Surface tension at a gas-liquid interface is produced by the attraction between the molecules being directed away from the surfaces of anode electrode 60 and cathode electrode 62 as the surface molecules are more attracted to the molecules within the water than they are to molecules of the gas at the electrode surfaces. In contrast, molecules of the bulk of the water are equally attracted in all directions. Thus, in order to increase the possible interaction energy, surface tension causes the molecules at the electrode surfaces to enter the bulk of the liquid.

In the embodiments in which gas-phase nanobubbles are generated, the gas contained in the nanobubbles (i.e., bubbles having diameters of less than about one micrometer) are also believed to be stable for substantial durations in the feed water, despite their small diameters. While not wishing to be bound by theory, it is believed that the surface tension of the water, at the gas/liquid interface, drops when curved surfaces of the gas bubbles approach molecular dimensions. This reduces the natural tendency of the nanobubbles to dissipate.

Furthermore, nanobubble gas/liquid interface is charged due to the voltage potential applied across membrane 58. The charge introduces an opposing force to the surface tension, which also slows or prevents the dissipation of the nanobubbles. The presence of like charges at the interface reduces the apparent surface tension, with charge repulsion acting in the opposite direction to surface minimization due to surface tension. Any effect may be increased by the presence of additional charged materials that favor the gas/liquid interface.

The natural state of the gas/liquid interfaces appears to be negative. Other ions with low surface charge density and/or high polarizability (such as Cl⁻, ClO⁻, HO₂⁻, and O₂⁻) also favor the gas/liquid interfaces, as do hydrated electrons. Aqueous radicals also prefer to reside at such interfaces. Thus, it is believed that the nanobubbles present in the catholyte (i.e., the water flowing through cathode chamber 56) are negatively charged, but those in the anolyte (i.e., the water flowing through anode chamber 54) will possess little charge (the excess cations cancelling out the natural negative charge). Accordingly, catholyte nanobubbles are not likely to lose their charge on mixing with the anolyte.

Additionally, gas molecules may become charged within the nanobubbles (such as O₂⁻), due to the excess potential on the cathode, thereby increasing the overall charge of the nanobubbles. The surface tension at the gas/liquid interface of charged nanobubbles can be reduced relative to uncharged nanobubbles, and their sizes stabilized. This can be qualitatively appreciated as surface tension causes surfaces to be minimized, whereas charged surfaces tend to expand to minimize repulsions between similar charges. Raised temperature at the electrode surface, due to the excess power loss over that required for the electrolysis, may also increase nanobubble formation by reducing local gas solubility.

As the repulsion force between like charges increases inversely as the square of their distances apart, there is an increasing outwards pressure as a bubble diameter decreases. The effect of the charges is to reduce the effect of the surface tension, and the surface tension tends to reduce the surface whereas the surface charge tends to expand it. Thus, equilibrium is reached when these opposing forces are equal. For example, assuming the surface charge density on the inner surface of a gas bubble (radius r) is Φ(e⁻/meter²), the outwards pressure (“Pₒᵤₜ”), can be found by solving the NavierStokes equations to give:

Pₒᵤₜ=Φ²/2D∈₀ (Equation 1)

where “D” is the relative dielectric constant of the gas bubble (assumed unity), “∈₀” is the permittivity of a vacuum (i.e., 8.854 pF/meter). The inwards pressure (“Pᵢₙ”) due to the surface tension on the gas is:

Pᵢₙ=2g/rPₒᵤₜ (Equation 2)

where “g” is the surface tension (0.07198 Joules/meter² at 25° C.). Therefore if these pressures are equal, the radius of the gas bubble is:

r=0.28792∈₀/Φ². (Equation 3)

Accordingly, for nanobubble diameters of 5 nanometers, 10 nanometers, 20 nanometers, 50 nanometers, and 100 nanometers the calculated charge density for zero excess internal pressure is 0.20, 0.14, 0.10, 0.06 and 0.04 e⁻/nanometer² bubble surface area, respectively, for example. Such charge densities are readily achievable with the use of an electrolysis cell (e.g., electrolysis cell 18). The nanobubble radius increases as the total charge on the bubble increases to the power ⅔. Under these circumstances at equilibrium, the effective surface tension of the liquid at the nanobubble surface is zero, and the presence of charged gas in the bubble increases the size of the stable nanobubble. Further reduction in the bubble size would not be indicated as it would cause the reduction of the internal pressure to fall below atmospheric pressure.

In various situations within the electrolysis cell (e.g., electrolysis cell 18), the nanobubbles may divide into even smaller bubbles due to the surface charges. For example, assuming that a bubble of radius “r” and total charge “q” divides into two bubbles of shared volume and charge (radius r½=r/2^{1/3}, and charge q_{1/2}=q/2), and ignoring the Coulomb interaction between the bubbles, calculation of the change in energy due to surface tension (ΔE_{sT}) and surface charge (ΔE_{q}) gives:

$$\begin{matrix} Δ {E}_{ST} = + 2 ( 4 πγ {r}_{1 / 2}^{2} ) - 4 πγ {r}^{2} = 4 πγ {r}^{2} ( {2}^{1 / 3} - 1 ) & ( Equation 3 ) \\ and & \\ Δ {E}_{q} = - 2 [ \frac{1}{2} \times \frac{{[ q / 2 ]}^{2}}{4 {πɛ}_{0} {r}_{1 / 2}} ] - \frac{1}{2} \times \frac{{q}^{2}}{4 {πɛ}_{0} r} = \frac{{q}^{2}}{8 {πɛ}_{0} r} [ 1 - {2}^{- 2 / 3} ] & ( Equation 4 )\end{matrix}$$

The bubble is metastable if the overall energy change is negative which occurs when ΔE_{ST}+ΔE_{q} is negative, thereby providing:

$$\frac{{q}^{2}}{8 {πɛ}_{0} r} [ 1 - {2}^{- 2 / 3} ] + 4 πγ {r}^{2} [ {2}^{1 / 3} - 1 ) \leq 0 \qquad ( Equation 5 )$$

which provides the relationship between the radius and the charge density (Φ):

$$Φ = \frac{q}{4 π {r}^{2}} \geq \sqrt{\frac{2 {γɛ}_{0}}{r} \frac{[ {2}^{1 / 3} - 1 ]}{[ 1 - {2}^{- 2 / 3} ]}} \qquad ( Equation 6 )$$

Accordingly, for nanobubble diameters of 5 nanometers, 10 nanometers, 20 nanometers, 50 nanometers, and 100 nanometers the calculated charge density for bubble splitting 0.12, 0.08, 0.06, 0.04 and 0.03 e⁻/nanometer² bubble surface area, respectively. For the same surface charge density, the bubble diameter is typically about three times larger for reducing the apparent surface tension to zero than for splitting the bubble in two. Thus, the nanobubbles will generally not divide unless there is a further energy input.

The above-discussed gas-phase nanobubbles are adapted for example to attach to dirt particles, thereby transferring their ionic charges. The nanobubbles stick to hydrophobic surfaces, which are typically found on typical dirt particles, which releases water molecules from the high energy water/hydrophobic surface interface with a favorable negative free energy change. Additionally, the nanobubbles spread out and flatten on contact with the hydrophobic surface, thereby reducing the curvatures of the nanobubbles with consequential lowering of the internal pressure caused by the surface tension. This provides additional favorable free energy release. The charged and coated dirt particles are then more easily separated one from another due to repulsion between similar charges, and the dirt particles enter the solution as colloidal particles.

Furthermore, the presence of nanobubbles on the surface of particles increases the pickup of the particle by micron-sized gas-phase bubbles, which may also be generated during the electrochemical activation process. The presence of surface nanobubbles also reduces the size of the dirt particle that can be picked up by this action. Such pickup assist in the removal of the dirt particles from floor surfaces and prevents re-deposition. Moreover, due to the large gas/liquid surface area-to-volume ratios that are attained with gas-phase nanobubbles, water molecules located at this interface are held by fewer hydrogen bonds, as recognized by water's high surface tension. Due to this reduction in hydrogen bonding to other water molecules, this interface water is more reactive than normal water and will hydrogen bond to other molecules more rapidly, thereby showing faster hydration.

For example, at 100% efficiency a current of one ampere is sufficient to produce 0.5/96,485.3 moles of hydrogen (H₂) per second, which equates to 5.18 micromoles of hydrogen per second, which correspondingly equates to 5.18×22.429 microliters of gas-phase hydrogen per second at a temperature of 0° C. and a pressure of one atmosphere. This also equates to 125 microliters of gas-phase hydrogen per second at a temperature of 20° C. and a pressure of one atmosphere. As the partial pressure of hydrogen in the atmosphere is effectively zero, the equilibrium solubility of hydrogen in the electrolyzed solution is also effectively zero and the hydrogen is held in gas cavities (e.g., macrobubbles, microbubbles, and/or nanobubbles).

Assuming the flow rate of the electrolyzed solution is 0.12 U.S. gallons per minute, there is 7.571 milliliters of water flowing through the electrolysis cell each second. Therefore, there are 0.125/7.571 liters of gas-phase hydrogen within the bubbles contained in each liter of electrolyzed solution at a temperature of 20° C. and a pressure of one atmosphere. This equates to 0.0165 liters of gas-phase hydrogen per liter of solution less any of gas-phase hydrogen that escapes from the liquid surface and any that dissolves to supersaturate the solution.

The volume of a 10 nanometer-diameter nanobubble is 5.24×10⁻²² liters, which, on binding to a hydrophobic surface covers about 1.25×10⁻¹⁶ square meters. Thus, in each liter of solution there would be a maximum of about 3×10⁻¹⁹ bubbles (at 20° C. and one atmosphere) with combined surface covering potential of about 4000 square meters. Assuming a surface layer just one molecule thick, for example, this provides a concentration of active surface water molecules of over 50 millimoles. While this concentration represents an exemplary maximum amount, even if the nanobubbles have greater volume and greater internal pressure, the potential for surface covering remains large. Furthermore, only a small percentage of the dirt particles surfaces need to be covered by the nanobubbles for the nanobubbles to have a cleaning effect.

Accordingly, the gas-phase nanobubbles, generated during the electrochemical activation process, are beneficial for attaching to dirt particles so transferring their charge. The resulting charged and coated dirt particles are more readily separated one from another due to the repulsion between their similar charges. They will enter the solution to form a colloidal suspension. Furthermore, the charges at the gas/water interfaces oppose the surface tension, thereby reducing its effect and the consequent contact angles. Also, the nanobubbles coating of the dirt particles promotes the pickup of larger buoyant gas-phase macrobubbles and microbubbles that are introduced. In addition, the large surface area of the nanobubbles provides significant amounts of higher reactive water, which is capable of the more rapid hydration of suitable molecules.

### 4. Tubular Electrode Example

As mentioned above, the electrolysis cell 18 shown in FIG. 1 can have any suitable shape or configuration, such as those shown in FIGS. 2 and 3. The electrodes themselves can have any suitable shape, such as planar, coaxial plates, cylindrical rods, or a combination thereof.

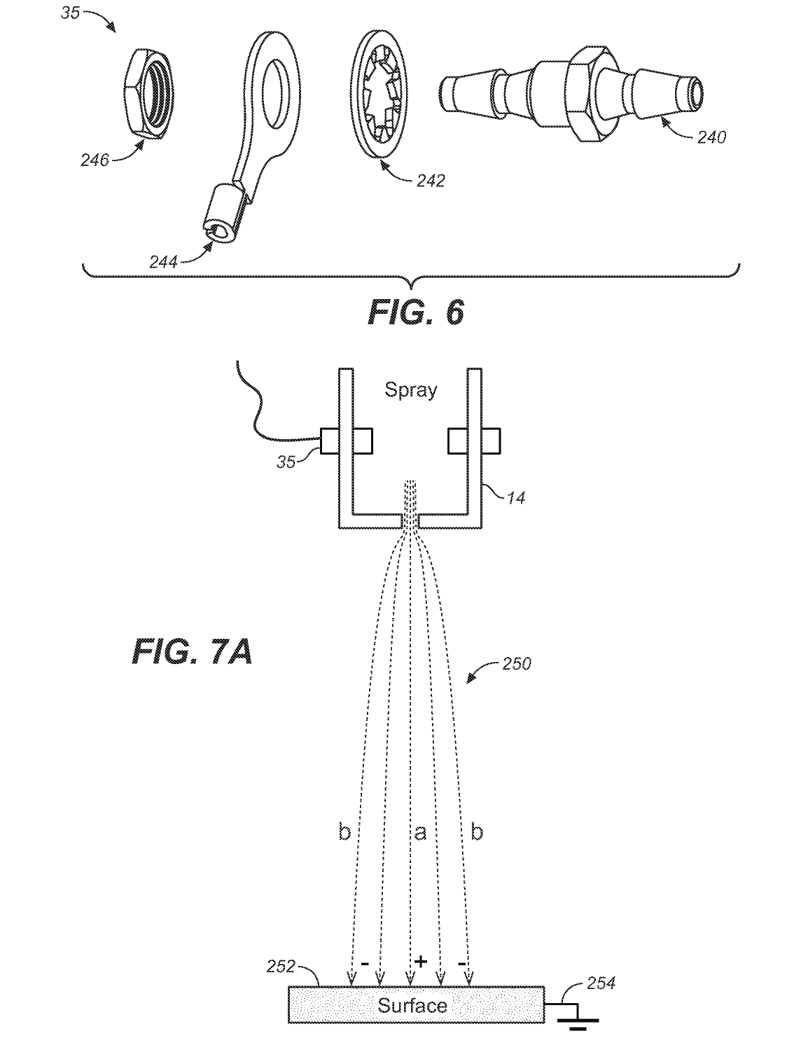

FIG. 5 illustrates an example of an electrolysis cell 200 having a tubular shape according to one illustrative example. For example, cell 200 can include the electrolysis cell contained in a hand-held spray bottle that is distributed by, and available from, a licensee of the assignee of this application, ActiveIon Cleaning Solutions, LLC of St. Josephs, Minn. under the name “Activeion™ Pro.”

Electrolysis cell 200 can be used in any of the embodiments disclosed herein, for example. The radial cross-section of cell 200 can have any shape, such as circular as shown in FIG. 5, or other shapes such as curvilinear shapes having one or more curved edges and/or rectilinear shapes. Specific examples include ovals, polygons, such as rectangles, etc.

Portions of cell 200 are cut away for illustration purposes. In this example, cell 200 is an electrolysis cell having a tubular housing 202, a tubular outer electrode 204, and a tubular inner electrode 206, which is separated from the outer electrode by a suitable gap, such as 0.040 inches. Other gap sizes can also be used, such as but not limited to gaps in the range of 0.020 inches to 0.080 inches. Either of the inner or outer electrode can serve as the anode/cathode, depending upon the relative polarities of the applied voltages.

An ion-selective membrane 208 is positioned between the outer and inner electrodes 204 and 206. In one example, outer electrode 204 and inner electrode 206 have conductive polymer constructions with apertures. However, one or both electrodes can have a solid construction in another example.

The electrodes 204 and 206 can be made from any suitable material, for example a conductive polymer, titanium and/or titanium coated with a precious metal, such as platinum, or any other suitable electrode material. In addition, multiple cells 200 can be coupled in series or in parallel with one another, for example.

In a specific example, at least one of the anode or cathode electrodes is formed of a metallic mesh, with regular-sized rectangular openings in the form of a grid. In one specific example, the mesh is formed of 0.023-inch diameter T316 (or, e.g. 304) stainless steel having a grid pattern of 20×20 grid openings per square inch. However, other dimensions, arrangements and materials can be used in other examples.

An ion-selective membrane 208 is positioned between the outer and inner electrodes 204 and 206. In one specific example, the ion-selective membrane includes a “NAFION” from E.I. du Pont de Nemours and Company, which has been cut to 2.55 inches by 2.55 inches and then wrapped around inner tubular electrode 206 and secured at the seam overlap with a contact adhesive, for example, such as a #1357 adhesive from 3M Company. Again, other dimensions and materials can be used in other examples. Other examples of suitable membranes include the other membranes described herein and, for example, those available from Membranes International Inc. of Glen Rock, N.J., such as the CMI-7000S cation exchange membrane and the AMI-7001S anion exchange membrane.

In this example, at least a portion of the volume of space within the interior of tubular electrode 206 is blocked by a solid inner core 209 to promote liquid flow along and between electrodes 204 and 206 and ion-selective membrane 208, in a direction along the longitudinal axis of housing 202. This liquid flow is conductive and completes an electrical circuit between the two electrodes. Electrolysis cell 200 can have any suitable dimensions. In one example, cell 200 can have a length of about 4 inches long and an outer diameter of about ¾ inch. The length and diameter can be selected to control the treatment time and the quantity of bubbles, e.g., nanobubbles and/or microbubbles, generated per unit volume of the liquid.

Cell 200 can include a suitable fitting at one or both ends of the cell. Any method of attachment can be used, such as through plastic quick-connect fittings. For example, one fitting can be configured to connect to the output tube 20 shown in FIG. 1. Another fitting can be configured to connect to the inlet filter 16 or an inlet tube, for example. In another example, one end of cell 200 is left open to draw liquid directly from reservoir 12 in FIG. 1.

In the example shown in FIG. 5, cell 200 produces anolyte EA liquid in the anode chamber (between one of the electrodes 204 or 206 and ion-selective membrane 208) and catholyte EA liquid in the cathode chamber (between the other of the electrodes 204 or 206 and ion-selective membrane 208). The anolyte and catholyte EA liquid flow paths join at the outlet of cell 200 as the anolyte and catholyte EA liquids enter tube 20 (in the example shown in FIG. 1). As a result, spray bottle 10 dispenses a blended anolyte and catholyte EA liquid through nozzle 14.

In one example, the diameters of tubes 20 and 22 are kept small so that once pump 24 and electrolysis cell 18 (e.g., cell 200 shown in FIG. 5) are energized, tubes 20 and 22 are quickly primed with electrochemically-activated liquid. Any non-activated liquid contained in the tubes and pump are kept to a small volume. Thus, in the embodiment in which the control electronics 30 activate pump and electrolysis cell in response to actuation of switch 28, spray bottle 10 produces the blended EA liquid at nozzle 14 in an “on demand” fashion and dispenses substantially all of the combined anolyte and catholyte EA liquid (except that retained in tubes 20, 22 and pump 24) from the bottle without an intermediate step of storing the anolyte and catholyte EA liquids. When switch 28 is not actuated, pump 24 is in an “off” state and electrolysis cell 18 is de-energized. When switch 28 is actuated to a closed state, control electronics 30 switches pump 24 to an “on” state and energizes electrolysis cell 18. In the “on” state, pump 24 pumps water from reservoir 12 through cell 18 and out nozzle 14.

Other activation sequences, configurations and arrangements can also be used. For example, control circuit 30 can be configured to energize electrolysis cell 18 for a period of time before energizing pump 24 in order to allow the feed water to become more electrochemically activated before dispensing.

The travel time from cell 18 to nozzle 14 can be made very short. In one example, spray bottle 10 dispenses the blended anolyte and catholyte liquid within, e.g., a very small period of time from which the anolyte and catholyte liquids are produced by electrolysis cell 18. For example, the blended liquid can be dispensed within time periods such as within 5 seconds, within 3 seconds, and within 1 second of the time at which the anolyte and catholyte liquids are produced.

If desired, further structures of one or more particular non-limiting examples of the tubular electrolysis cell 200 are shown and described in Field U.S. patent application Ser. No. 12/488,360, filed Jun. 19, 2009, which is hereby incorporated by reference in its entirety. These structures can be used in any of the embodiments disclosed herein and modifications thereof.

### 5. Additional High-Voltage Electrode Enhancing Santitization Properties of Electrolyzed Output Example

While the electrolyzed liquid produced by an electrolysis cell may have enhanced cleaning properties, it may be desired to further enhance the sanitizing properties of the anolyte, catholyte and/or combined anolyte/catholyte liquid that is produced by the cell.

For example, depending on the characteristics of the voltage applied to the electrolysis cell and the properties of the liquid (e.g., tap water) fed to the cell, the chemical properties of the liquid produced by the cell may not be sufficient to produce consistent sanitizing properties. While the electrolysis process produces certain amounts of hydrochlorous acid, which can have sanitizing properties, typical electrolysis processes rely on “salt doping” to effect charge transfer through the liquid, and there can be inconsistent “salts” in tap water. This can lead to unpredictable concentrations of hydrochlorous acid and unpredictable sanitizing properties.

It has been found that in one or more of the embodiments of the present disclosure that the electrodes in the electrolysis cell generate, e.g., a small electrical charge in the liquid. It has also been found that liquid path from the electrolysis cell to the surface or volume being treated by the output spray can be electrically conductive, relative to Earth ground, for example. The electrical potential between one or more of the cell electrodes and Earth ground can enhance sanitization of microorganisms on the surface or in the volume contacted by the liquid.

The electrical potential is applied e.g. through the liquid and/or liquid/gas mixture to the microorganisms and, if the resulting electric field applied across the cells of the microorganism is of a sufficient magnitude, the electric field can cause irreversible damage or destruction to the microorganisms through a mechanism such as electroporation and/or elecrohydraulic shock, as discussed in more detail below.

In an illustrative embodiment of the present disclosure, the electrical charge delivered through the liquid dispensed by the hand-held device shown in FIG. 1 can be further enhanced by a separate electrical conductor, lead, or other electrical and/or electromagnetic component, for example, an electrode, e.g., high voltage (in a relative sense) electrode 35, to impart, apply, induce or otherwise cause an electrical potential in a liquid output spray and/or stream. In the example shown in FIG. 1, electrode 35 is positioned in the liquid path to cause a separate, greater electrical potential relative to Earth ground, as compared to the potential generated by electrolysis cell 18, for example. Also in the example shown in FIG. 1, electrode 35 is positioned along tube 22. However, electrode 35 can be located at any position along the liquid flow path from reservoir 12 to nozzle 14 (or even external to spray bottle 10) or other position as appropriate, e.g., to conduct electrical charge to charge or additionally charge liquid dispensed by the hand-held device.

In one example, electrode 35 is formed by an electrically conductive spike or “barb”, which is inserted through the side wall of tube 22 so a portion of the electrode comes into physical contact with liquid flowing through tube 22. In another example, tube 22 is made at least partially of an electrically conductive material, such as a metal and/or a conductive polymer. For example, tube 22 can include a section made of copper, which is electrically connected to an electrical lead extending from control electronics 30. In an exemplary embodiment, the additional electrode 35 is separate from and external to electrolysis cell 18 and has no corresponding return electrode (e.g., an electrode of opposite polarity and/or an electrode representing a circuit ground for the electroporation electrode). It will be appreciated that other arrangements in other embodiments may be utilized.

The power supply on control electronics 30 can be configured to deliver an AC and/or DC voltage (such as a positive voltage) to lead 35 and thus to the liquid in tube 22. Tube 22 is configured to conduct electricity from lead 35 to liquid being delivered through the tube and thus apply an electrical potential and/or additional electrical potential to liquid entering nozzle 14. This additional electrical potential can increase the electroporation/electrohydraulic shock inflicted on the microorganisms, for example.

Various voltages and voltage patterns can be used in alternative embodiments. Earth ground serves to complete the electrical circuit formed by electrode 35, the liquid stream delivered by nozzle 14, and the surface or volume to which the stream is applied.

The additional voltage (and/or current) can be applied at any location along the flow path of bottle 10, from reservoir 12 to the output of nozzle 14 (or externally to bottle 10) for example. For example, if nozzle 14 is at least partially conductive, lead 35 can be coupled to nozzle 14. In other examples, lead 35 is electrically coupled to a probe tip that is in contact with the liquid at any location along the flow path. In another example, lead 35 is electrically coupled to the housing of pump 24, which, if conductive, delivers the electrical charge to the liquid passed through the pump. In yet a further example, the lead 35 can deliver additional electrical charge to liquid contained within electrolysis cell 18. In yet a further example, the electrolysis cell 18 is eliminated from bottle 10, wherein liquid sprayed from nozzle 14 is not electrochemically activated but can still carry an electrical charge as a result of a conductor such as lead 35 for causing electroporation/electrohydraulic shock.

### 5.1 Example High

### Voltage, Electroporation Electrode

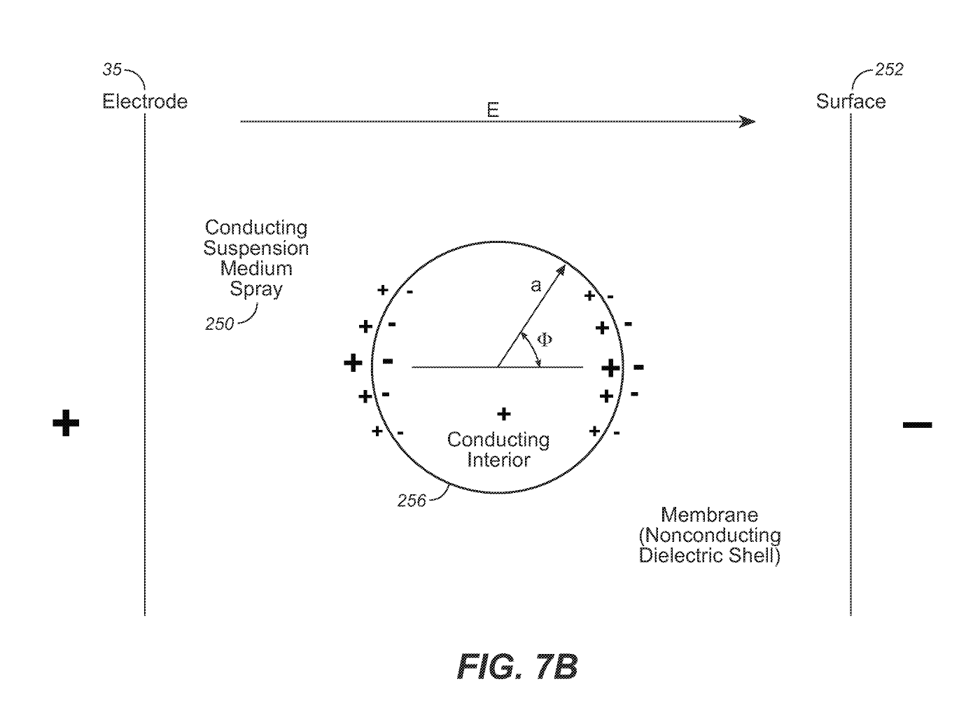

FIG. 6 is an exploded view of a high-voltage electroporation electrode 35 according to an illustrative embodiment of the disclosure. Electrode 35 includes an adapter 240, a washer 242, a terminal 244 and a nut 246. Adapter 240 has two opposing ends with male connectors (e.g., barbs) for connecting between two sections of tube 22 (shown in FIG. 1), for example. Adapter 240 has an internal lumen for passing liquid from one end to the other, along the liquid flow path of the apparatus. Adapter 240 can be formed of any suitable material, such as an electrically-conductive material, such as copper, brass, and/or silver. In one particular embodiment, at least a portion of adapter 240 is formed of or coated with silver. For example, adapter 240 can be formed of brass, wherein at least a portion of the surface in contact with the liquid is coated with silver. For example, the internal and external diameter surfaces are coated with silver.

Nut 246 threads onto one end of adapter 240, thereby holding terminal 244 and washer 244 in tight electrical contact with the adapter. An electrical lead (not shown) can be attached to terminal 244 for electrically connecting the terminal with the control electronics 30 (shown in FIG. 1). Since adapter 240 is electrically conductive, the potential applied to adapter 240, through terminal 244, is applied to the liquid flowing through the adapter, relative to the surface being sprayed.

In another embodiment, electrode 35 is formed by an electrically conductive spike, which extends through a sidewall of tube 22 such that the spike makes electrical contact with liquid flowing through the tube. Other configurations can also be used.

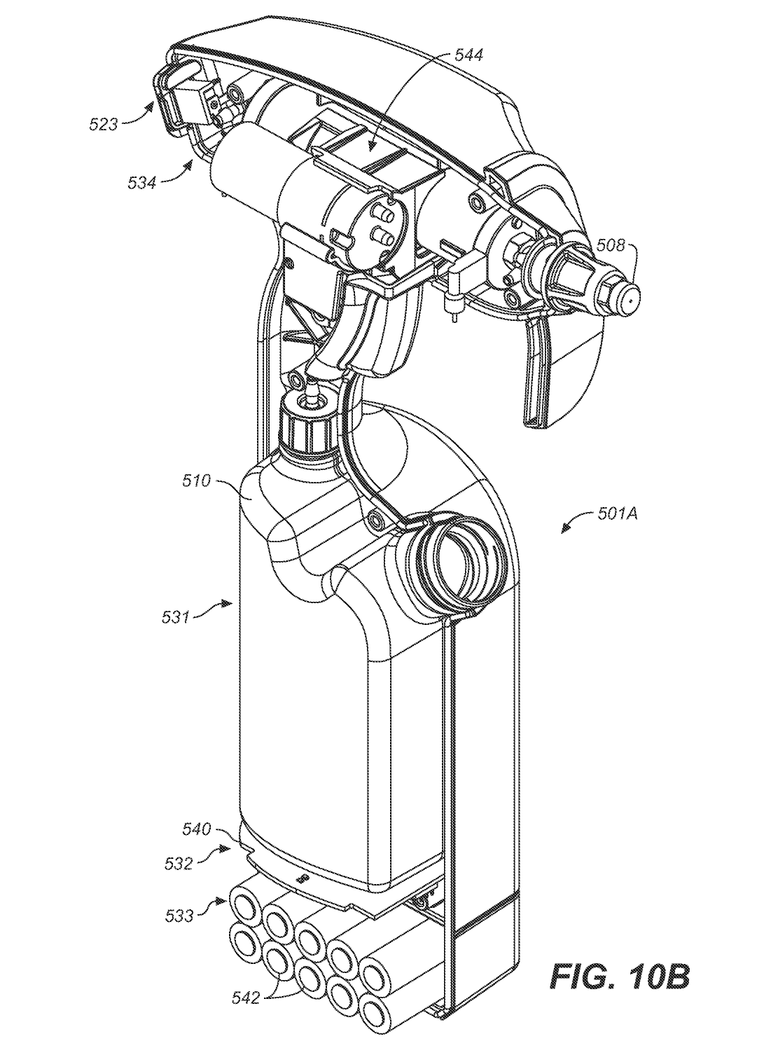

In yet another embodiment, the electrode can be formed by an electrically conductive nozzle. For example, nozzle 14 in FIG. 1 or nozzle 508 in FIG. 10A can be formed of an at least partially conductive material, such as but not limited to, silver-coated brass.

The silver plating may also enhance the sanitization action. Silver may provide good electrical conductivity with the liquid flowing along the flow path. It is also possible that, when an electrical potential is applied to electrode 35 and a current flows from electrode 35 to the surface through the liquid output spray, silver ions can migrate from the electrode into the liquid flow. Silver ions are known to have a toxic effect on some bacteria, viruses, algae and fungi. Therefore, use of a silver electrode can further enhance the sanitization properties of the dispensed liquid and/or liquid/gas mixture.

### 5.2 Electroporation Mechanism Example

The following discussion is provided as an example only and not intended to limit the present disclosure, operation of examples described herein and/or the scope of any issued claims appended hereto.

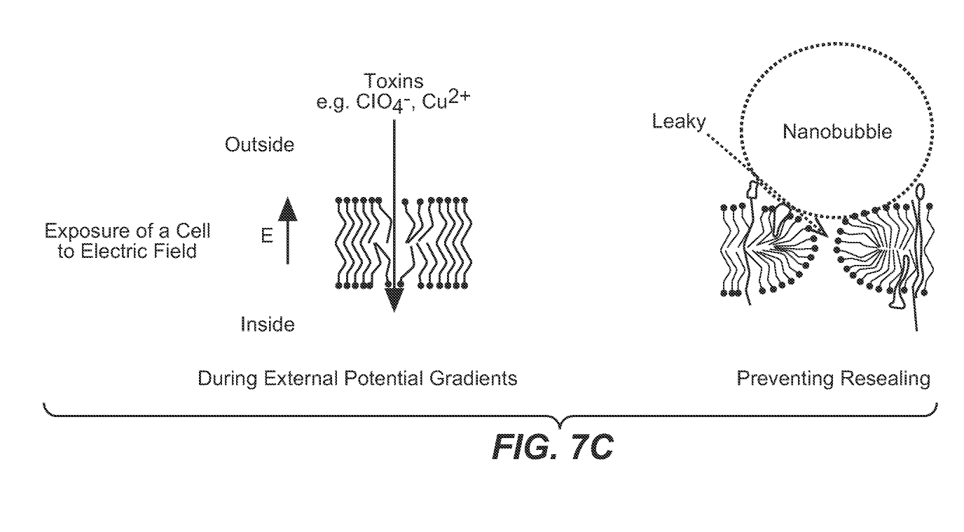

FIG. 7A is a diagram illustrating the spray output 250 from spray nozzle 14, wherein individual droplets may take different paths, e.g., “a” and “b” from the nozzle to the surface 252 being treated. Surface 252 may or may not have an electrical conduction path to ground 254, such as Earth ground.

FIG. 7B is a diagram illustrating an example of the electroporation mechanism achieved by spraying surface 252 (in FIG. 7A) with output spray 250 from spray bottle 10 shown in FIG. 1. The output spray 250 dispensed on surface 35 has been found to form a conducting suspension medium. FIG. 7B illustrates the resulting electric field “E” applied to a cell membrane 256 of a microorganism that is suspended from surface 252 by the dispensed liquid from output spray 250. The output spray 250 and the liquid dispensed on surface 252 together form a conductive path from electrode 35 to surface 252, for example. The addition of an applied alternating potential from electrode 35 to the electrolytic water spray appears to endow the output spray 250 with significantly enhanced sanitizing action. This phenomenon has been associated with irreversible electroporation. In one particular embodiment, the alternating potential appears to be particularly effective at 600 V, 28 kHz with a variable effect for different organisms. However, other voltage and frequencies can be used in other embodiments.

Electroporation followed by cell death is known to be achievable with a transmembrane potential of at least 0.5 V (where a membrane thickness is typically ˜3 nm, for example). Depending on the configuration, such potentials may require a pulse of about 10 kV/cm or more. Lower potentials may be effective, for example in the presence of cell toxins or with the availability of additional mechanisms for preventing normally reversibly-formed pores from resealing. It should be noted that although electroporation is commonly used as a ‘reversible’ tool at lower potentials, it is recognized that, even under these conditions, often only a small percentage of cells recover.

The formation of holes in the cell membranes is generally insufficient in itself to cause cell death, as it is known that cells can survive for relatively long periods with large amounts of membrane missing.

Cell death comes because of disruption to the metabolic state of the cells, which can be caused by electrophoretic and electroosmotic (capillary electrophoretic) movement of materials into and out of the cells. Diffusion by itself is generally too slow. To achieve electrophoresis and electroosmosis, sufficient power must be dissipated within the surface, as shown in the diagram of FIG. 7C.

Different microorganisms have different total surface charges and charge distributions and therefore will react differently to each other in terms of cell death. They will also behave differently in the oscillating potential field and will have different resonant frequencies for maximum absorption (and hence maximum movement relative to the aqueous solution, causing the maximum chaos to their metabolism). Movement in and out depends primarily on potential gradients. Increased effects occur when the system is in resonance.

When considering the potential gradient delivered to the cell and the power dissipated to the sprayed surface, in one particular example, the spray device delivers a fine spray that may be partially a true aerosol (˜1μ droplets), but mostly a mist with droplet sizes much greater than 10μ. The droplet sizes and velocity profiles can vary between different embodiments.

The velocity of the liquid exiting the nozzle is simply calculated from the rate of liquid sprayed divided by the area of the exit orifice. However the subsequent decrease in droplet speed depends on the droplet size (mass to surface area ratio). The terminal velocity of 10μ and 50μ droplets are only about 10⁻³ m/s and 10⁻¹ m/s respectively.

Sprayed water droplets descend at different rates, and the time differences will be significant when related to the rapidly alternating potential (e.g., 28 kHz). For example, in FIG. 7A, pathway (b) will be longer than pathway (a), for example by about 1 cm. The descent velocity (dependent on the drop size, flow rate and nozzle diameter) will determine the difference in time between the drops landing but this is likely to be several to many times the potential cycling time of 36 μs.

If the potential is determined by the time of descent, then significant potential gradients will exist within the two dimensional surface with greater field gradients towards the periphery of the sprayed field. A droplet just 1 cm out from the center still travels an additional about 0.03 cm and, even if travelling at 10 m/s, this is equivalent to one cycle of the potential. These potential gradients might exist if the drops are not in effectively continuous contact with the sprayer electrode. If all the spray has the same potential on impinging the surface in spite of the different routes taken (and consequent times of descent) of the droplets, then the potential gradients are not within the surface as such but between the surface and ‘earth’ and these may not be sufficient to cause electroporosity if the surface is not ‘earthed’.

Cells with open pores are much more prone to the effects of cell toxins in the aqueous solution as they have no barrier to their entry. The potential cell toxins co-delivered with the alternating potential are peroxide, chlorine oxides, and other redox agents such as superoxide, ozone and singlet oxygen, and heavy metal ions such as cupric ions and/or silver ions.

Charged nanobubbles will move in the electric fields and will be capable of picking up materials from the surface. As they are surface-active, they may additionally interfere with pore resealing and preferentially deliver their cytotoxic surface active molecules to the pore sites, as shown in FIG. 7C, for example.

In view of the above, the electrolyzed water produced by spray bottle 10, shown in FIG. 1, for example, acts as a cleaning agent due to production of tiny electrically-charged bubbles. These attach themselves to dirt particles/microorganisms so transferring their charge. The charged and coated particles separate one from another due to the repulsion between their similar charges and enter the solution as a suspension. Coating of the dirt by tiny bubbles promotes their pick-up by larger buoyant bubbles that are introduced during cleaning, thus aiding the cleaning process. Simultaneously, microorganisms can be electroporated and killed or otherwise eliminated by the electric potential generated by the additional electrode 35, e.g. reducing the number of microorganisms on a surface.

Thus, to enhance sanitization ability properties, electroporation can be used for example to accomplish a more consistent and effective destruction of microbial action by discharging (in a relative sense) a high-voltage to a ground (such as Earth ground) through e.g. an aqueous fluid.

It has also been found that the combination of the electrochemically-activated liquid produced by the electrolysis cell and the electric field applied by the electroporation electrode has a synergistic effect. It is believed that as the charged nanobubbles produced in the electrochemically-activated liquid move in the electric fields, they pick up microorganisms and separate them from the surface. By separating the microorganisms from the surface, such that they are suspended in the liquid on the surface, the electric field produced along the surface by the electroporation electrode is applied more easily across the microorganism cells. Whereas, if the microorganism is in contact with the surface, the electric field is more easily discharged into the surface ground and may be less effective in creating irreversible electroporation of the organisms cells. With the cell suspended, the applied alternating field oscillates back and forth causing damage to the cells.

In alternative embodiments, microorganism suspension can be accomplished through mechanisms other than electrochemically-activated liquids produced by electrolysis cells. For example, the microorganisms can be suspended by using a detergent and/or mechanical action or combination. Particular examples of other suspension mechanisms include, for example, any mechanism that alters the ORP of the dispensed liquid (producing dispensed liquid having a positive ORP, a negative ORP or a combination of both). For example, it has been found that regular tap water may be altered to have a negative ORP (such as but not limited to −50 millivolts to −600 millivolts) which has enhanced cleaning effects. These enhanced cleaning effects can serve to suspend microorganisms above the surface within the dispensed liquid, for example. Although negative (and/or positive) ORP can be achieved through an electrolysis cell as described herein, it can also be achieved by other mechanisms such as by use of surfactants (and/or detergents carrying surfactants), and/or by passing the liquid to be dispensed through a filter or other mechanism containing a material, such as zeolites, that alters the ORP of the liquid.

As describe in more detail herein, zeolites, depending on the type, can impart a negative ORP (and/or a positive ORP) on liquids such as regular tap water by ion exchange. Thus, in one or more of the embodiments disclosed herein, the electrolysis cell is replaced for example by a zeolite filter, or a zeolite filter is used in combination with an electrolysis cell. Such a filter can be positioned for example anywhere along the liquid flow and/or within the source liquid container. Other materials or mechanisms suitable for ion exchange, such as a resin or other matrices, may be utilized in other embodiments depending on their ability to impart an altered ORP.

The electroporation electrode may also be used (such as in the various embodiments disclosed herein) in combination with other wet cleaning technologies, such as a chemical-based system that use a chemical within the dispensed liquid for inactivating microorganisms, with or without use of an electrolysis cell. These chemical based wet cleaning technologies might provide longer residence times and thus greater sanitizing effect on some surfaces, such as porous surfaces, for example.

### 5.3 Electroporation by Hand-Held Spray Bottle Example

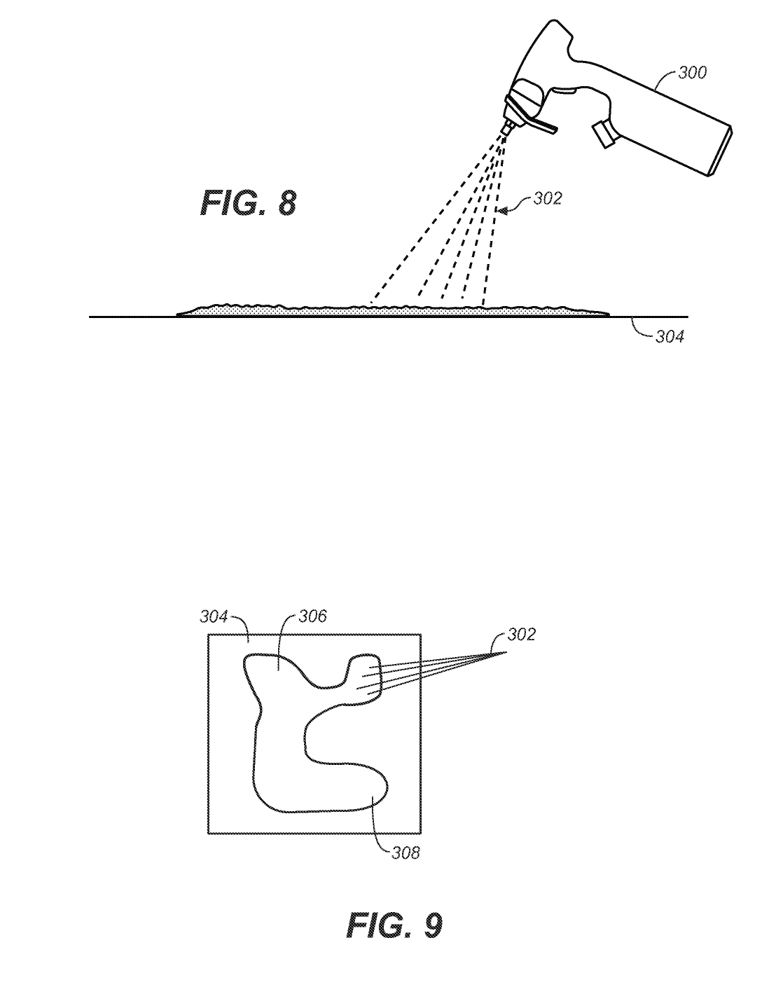

In the example shown in FIG. 8, an aspect of the disclosure relates to a process for deactivating or destroying microorganisms, by applying a potential or electrochemical pressure to microorganisms, in a charged medium such as an atomized spray generated by an electrolysis cell carried by a hand-held spray apparatus 300. However, spray bottle 300 can be replaced with any other apparatus or system having an electrolysis cell and a high-voltage electroporation electrode as described herein.

As shown in FIG. 8, the spray nozzle of the hand-held spray bottle 300 dispenses the electrochemically-activated liquid as a charged output spray 302, which forms an electrically-coupled conduit of spray. As the output spray 302 contacts a surface 304, the electrical conduit of spray 302 becomes electrically coupled to the surface, thus completing an electrically conductive path from the cell electrodes and the high-voltage electroporation electrode to the surface. This path allows electrical charge to be delivered to microorganisms present on the surface.

Further, it has been found that as the surface becomes wet with the liquid carried by the output spray, the electrical charge conducts throughout and along the wetted surface, as long as there exists a conductive path of liquid between the output spray and various areas on the surface that are remote from direct contact by the output spray. It has been found that an electrical charge can be measured at an area remote from direct contact by the output spray if the surface has a continuous path of liquid between the area of direct contact an the remote area at which the measurement is made.

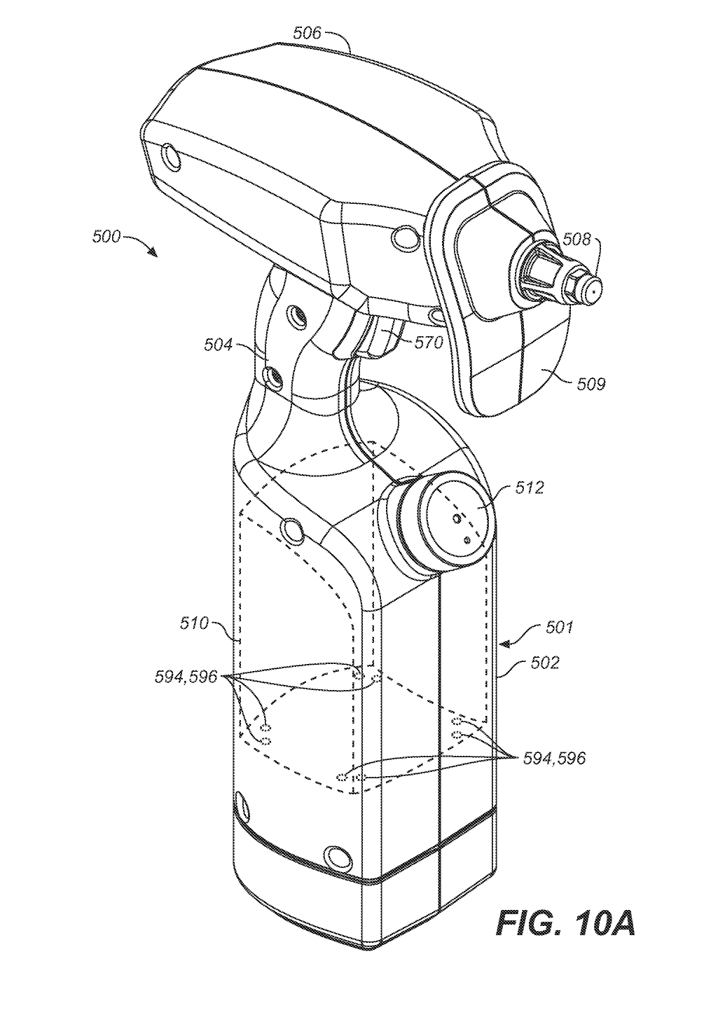

For example, FIG. 9 illustrates a plan view of partially wetted surface 304. As spray 302 contacts surface 304, the liquid carried by spray 302 forms a conductive path 306, which carries electrical charge from the output spray to remote area 308 that is not in direct contact with the output spray. This conductive path can serve to increase the length of time various areas of the surface are treated by the charge as the output spray is advanced along the surface.

In one aspect of the disclosure, spray bottle 300 (or other liquid delivery apparatus) is configured and operated to deliver an electrical charge through the output liquid in a manner that results in a delivered charge magnitude that exceeds a limit of intracellular and extracellular electrostatic capacity possessed by one or more microorganisms on the surface being treated. In one example, the apparatus is configured and operated to achieve a transmembrane potential of at least 0.5 Volts on cells of one or more of the microorganisms on the surface that are in contact with the liquid dispensed from the apparatus.

### 6. Particular Spray Bottle Example

### 6.1 Bottle Configuration Example

FIG. 10A illustrates a specific example of a commercial embodiment of the spray bottle shown schematically in FIG. 1. The particular bottle configurations and constructions shown in the drawings are provided as non-limiting examples only.

If desired, further structures of one or more particular non-limiting examples of spray bottle 500 are shown and described in Field U.S. patent application Ser. No. 12/488,368, filed Jun. 19, 2009, which is hereby incorporated by reference in its entirety. These structures can be used in any of the embodiments disclosed herein and modifications thereof.

A commercial embodiment is presently available in a hand-held spray bottle form, which is distributed by, and available from, ActiveIon Cleaning Solutions, LLC of St. Josephs, Minn. under the name “Activeion™ Pro.” The embodiment in the example shown in FIGS. 10A-10C is similar to the foregoing spray bottle with a modification regarding addition of an electroporation electrode and related control circuitry, etc.

In FIG. 10A, bottle 500 includes a housing 501 forming a base 502, a neck 504, and a barrel or head 506. The tip of barrel 506 includes a nozzle 508 and a drip/splash guard 509. In one example, nozzle 508 is formed of brass. Drip/splash guard 509 also serves as a convenient hook for hanging bottle 500 on a utility cart, for example. Housing 501 has a clamshell-type construction with substantially symmetrical left and right hand sides attached together, such as by screws. Base 502 houses a container 510, which serves as a reservoir for liquid to be treated and then dispensed through nozzle 508. Container 510 has a neck and threaded inlet (with a screw cap) 512 that extends through base 502 to allow container 510 to be filled with a liquid. Inlet 512 is threaded to receive a cap seal.

In this example, the entire housing or a portion of the housing is at least translucent. Similarly, container 510 is formed of a material that is at least translucent. For example, container 510 can be fabricated as a blow mold of a clear polyester material. As explained in more detail below, housing 501 also contains a circuit board carrying a plurality of LED indicator lights 594, 596. In this example, there are four red LEDs 594 and four green LEDs 596 (also shown in phantom), arranged in pairs in each corner of the bottle. The lights are positioned beneath the base of container 510 to transmit light through a base wall of container 510 and into any liquid contained in the container. The liquid diffuses at least a portion of the light, giving an appearance of the liquid being illuminated. The color of the light and/or other illumination characteristics such as on/off modulation, intensity, etc. that are controlled by the control electronics are observable from an exterior of the bottle to give the user an indication of the functional status of the bottle.

For example, the liquid can be illuminated with green LEDs to indicate that the electrolysis cell and/or pump are functioning properly. Thus, the user can be assured that the treated liquid dispensed from nozzle 508 has enhanced cleaning and/or sanitizing properties as compared to the source liquid contained in container 510. Also, illumination of the source liquid in container 510, although not yet treated, gives an impression that the liquid is “special” and has enhanced properties.

Similarly, if the electrolysis cell and/or pump are not functioning properly, the control electronics illuminates the red LEDs, giving the source liquid a red appearance. This gives the user an impression that there is a problem and that the dispensed liquid may not have enhanced cleaning and/or sanitizing properties.

FIG. 10B illustrates various components installed in the left-hand side 501A of housing 501. Container 510 is installed in compartment 531, circuit board 540 is installed in compartment 532, batteries 542 are installed in compartment 533, and pump/cell assembly 544 is installed in compartment 534. The various tubes that connect container 510, pump/cell assembly and nozzle 508 are not shown in FIG. 10B.

The back end of the barrel (or head) 506 of bottle 501 includes an electrical power jack 523 for connecting to the cord of a battery charger (not shown). In the example in which bottle 500 carries rechargeable batteries, these batteries can be recharged through jack 523.

FIG. 10C illustrates a fragmentary, close-up view of pump/cell assembly 544 installed in the barrel 506 of housing half 501A. Pump/cell assembly 544 includes a pump 550 and an electrolysis cell 552 mounted within a bracket 554. Electrolysis cell 552 has an inlet 556 that is fluidically coupled to a tube (not shown) extending from the outlet of container 510 and an outlet 557 that is fluidically coupled through another tube (also not shown) to an inlet 555 of pump 550. Pump 550 has an outlet that is fluidically coupled to the inlet 558 of nozzle 508. In one example, electrolysis cell 552 corresponds to the tubular electrolysis cell 200 discussed with reference to FIG. 5. However, any suitable electrolysis cell in this and other embodiments disclosed herein, such as those disclosed in Field et al. U.S. Publication No. 2007/0186368 A1, including but not limited to the electrolysis cells (e.g., functional generators) disclosed in FIGS. 8A, 8B and 9. O-ring 560 provides a seal about the nozzle 508 for housing 501. Also, pump 550 can be located upstream or downstream of cell 552.

As described above with reference to FIG. 6, in this example, the high voltage electroporation electrode 35 is fluidically coupled between the outlet 557 of cell 552 and the inlet 558 of nozzle 508. The electrode adapter 240 (shown in FIG. 6) is spliced within a tube connecting outlet 557 and inlet 558 to provide an electrical connection to the fluid flowing to nozzle 508. However, the electrode 35 can be located at other locations along the fluid flow paths of bottle 500.

Bottle 500 further includes a trigger 570, which actuates a momentary push-button on/off switch 572. Trigger 570 actuates about pivot when depressed by a user. A spring (not visible in FIG. 10C) biases trigger 570 in a normally released state and thus switch 572 in an off state. Switch 572 has electrical leads for connecting to the control electronics on circuit board 540, shown in FIG. 10A.

When trigger 570 is depressed, switch 572 actuates to the “on” state, thereby providing electrical power to the control electronics, which energizes pump 550 and electrolysis cell 552. When energized, pump 550 draws liquid from container 510 and pumps the liquid through electrolysis cell 552 and Electroporation electrode adapter 240 (FIG. 6), which deliver a combined anolyte and catholyte EA liquid to nozzle 508. When pump 550 and/or electrolysis cell 552 are functioning properly, the control electronics also illuminate the green LEDs installed on the circuit board or another location in or on bottle 500.

In an exemplary embodiment, nozzle 508 maintains a fluid stream during use that is sufficient to conduct an electric field applied by the electroporation electrode 35 to the surface or volume of space being treated, through the dispensed liquid. With some nozzles, it has been found that the nozzle may cause cavitation of the liquid stream that may disrupt electrical conductivity along the output stream, thus potentially reducing the electric field applied to the surface being treated. Using an electrically conductive nozzle (such as brass, another metal, and/or conductive plastic) may help to maintain an electrical conductive path along the relevant or desired liquid path, e.g., from the electroporation electrode 35, through the nozzle, to the output spray that is delivered to the surface, even if some cavitation of the liquid occurs within the nozzle. An illustrative example of a suitable nozzle is a #TT276-1/8M-2 hydraulic atomizing nozzle from Spraying Systems Co., P.O. Box 7900 Wheaton, Ill. Also, this nozzle is used at a pressure of 25-40 psi, for example. Other types of nozzles and pressure ranges can be used in other examples.

When using a conductive nozzle, such as a brass nozzle, it may also be beneficial to insulate the outer surface of the nozzle, e.g., with a dielectric, such as by using a plastic cap over the nozzle, which has an aperture for the spray output. The plastic cap may limit an electrical discharge if the nozzle comes in contact with a conductive surface or a person's skin, for example.

### 6.2 Control Circuits Example

### 6.2.1 Driving Voltage for Electrolysis Cell Example

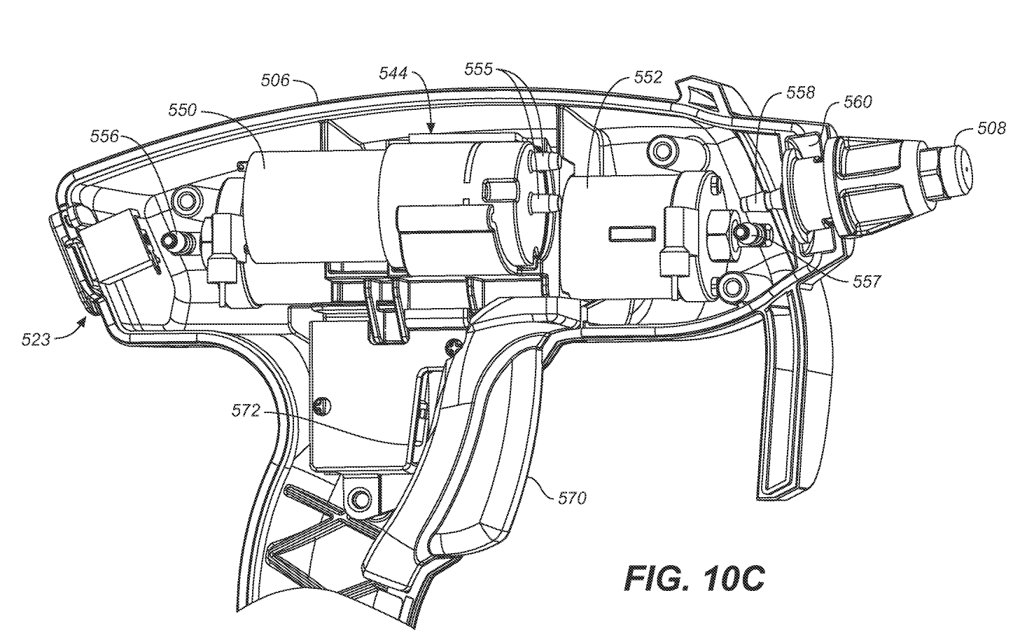

FIG. 11 is a waveform diagram illustrating the voltage pattern applied to the anode and cathode of electrolysis cell 552 (in the bottle shown in FIGS. 10A-10C) according to an exemplary aspect of the present disclosure. A substantially constant, relatively positive voltage is applied to the anode, while a substantially constant, relatively negative voltage is applied to the cathode. However, periodically each voltage is briefly pulsed to a relatively opposite polarity to repel scale deposits. In some examples, there is a desire to limit scale deposits from building on the electrode surfaces. In this example, a relatively positive voltage is applied to the anode and a relatively negative voltage is applied to the cathode from times t0-t1, t2-t3, t4-t5 and t6-t7. During times t1-t2, t3-t4, t5-t6 and t7-t8, the voltage applied to each electrode is reversed. The reversed voltage level can have the same magnitude as the non-reversed voltage level or can have a different magnitude if desired.

The frequency of each brief polarity switch can be selected as desired. As the frequency of reversal increases, the amount of scaling decreases. However, the electrodes may loose small amounts of platinum (in the case of platinum coated electrodes) with each reversal. As the frequency of reversals decreases, scaling may increase. In one example, the time period between reversals, as shown by arrow 300, is in the range of about 1 second to about 600 seconds. Other periods outside this range can also be used. In this example, the time period of normal polarity 303, such as between times t2 and t3, is at least 900 milliseconds.

The time period at which the voltages are reversed can also be selected as desired. In one example, the reversal time period, represented by arrow 302, is in the range of about 50 milliseconds to about 100 milliseconds. Other periods outside this range can also be used.

With these ranges, for example, each anode chamber produces a substantially constant anolyte EA liquid output, and each cathode chamber produces a substantially constant catholyte EA output without requiring valving.

In prior art electrolysis systems, complicated and expensive valving is used to maintain constant anolyte and catholyte through respective outlets while still allowing the polarity to be reversed to minimize scaling.

If the number of anode electrodes is different than the number of cathode electrodes, e.g., a ratio of 3:2, or if the surface area of the anode electrode is different than the surface area of the cathode electrode, then the applied voltage pattern can be used in the above-manner to produce a greater amount of either anolyte or catholyte in the produced liquid. With a tubular electrolysis cell 552 (such as cell 200 shown in FIG. 5), outer cylindrical electrode 204 has a greater diameter and therefore a greater surface area than inner cylindrical electrode 206. To emphasize enhanced cleaning properties, the control circuit can be configured, for example, to drives cell 200 so that, for a majority of period of the driving voltage pattern, outer electrode 204 (or the greater number of electrodes in embodiments having unequal numbers of anodes and cathodes) serves as the cathode and inner electrode 206 (or the lesser number of electrodes in embodiments having unequal numbers of anodes and cathodes) serves as the anode. Since the cathode has a larger surface area (or number of electrodes) than the anode, cell 200 will e.g. generate more catholyte than anolyte per unit of time through the combined outlet of the cell.

If sanitizing is to be emphasized, then outer electrode 204 (or the greater number of electrodes) can be driven to the relatively positive polarity (to produce more anolyte) and the inner electrode (or the lesser number of electrodes) can be driven to the relatively negative polarity (to produce less catholyte).

Referring to FIG. 11, in this example, the control circuit applies a relatively positive voltage to the anode (electrode 206) and a relatively negative voltage to the cathode (electrode 204) from times t0-t1, t2-t3, t4-t5 and t6-t7. During times t1-t2, t3-t4, t5-t6 and t7-t8, the voltages applied to each electrode is briefly reversed.

It has been found that such frequent, brief polarity reversals for de-scaling the electrodes may have a tendency also to shed materials often used for plating the electrodes, such as platinum, from the electrode surface. Thus in one embodiment, electrodes 204 and 206 comprise unplated electrodes, such as metallic electrodes or conductive plastic electrodes. For example, the electrodes can be unplated metallic mesh electrodes.

In one exemplary embodiment, the spray bottle (or other apparatus) can further include a switch that can be used to selectively invert the waveform shown in FIG. 11 (or any other waveform applied to the electrolysis cell). For example, the switch can be set in one position to generate more anolyte than catholyte and in another position to generate more catholyte than anolyte. The control circuit monitors the switch position and adjusts the voltage applied to the electrolysis cell according to the switch position.

However, the electrodes of the electrolysis cell can be driven with a variety of different voltage and current patterns, depending on the particular application of the cell.

In another example, the electrodes are driven at one polarity for a specified period of time (e.g., about 5 seconds) and then driven at the reverse polarity for approximately the same period of time. Since the anolyte and cathotlyte EA liquids are blended at the outlet of the cell, this process produces essentially one part anolyte EA liquid to one part catholyte EA liquid.

In another example, the cell electrodes are driven with a pulsed DC voltage waveform, wherein the polarity applied to the electrodes is not reversed. The “on/off” time periods and applied voltage levels can be set as desired.

### 6.2.2 Control Circuit for Electrolysis Cell Example

The waveform applied to the electrolysis cell is controlled by control circuit 30, shown in FIG. 1, which resides, for example, on circuit board 540 shown in FIG. 10B. Control circuit 30 can include any suitable control circuit and can be implemented in hardware, software, or a combination of both, for example.

Control circuit 30 includes a printed circuit board containing electronic devices for powering and controlling the operation of pump 24 and electrolysis cell 18. In one example, control circuit 30 includes a power supply having an output that is coupled to pump 24 and electrolysis cell 18 and which controls the power delivered to the two devices. Control circuit 30 also includes an H-bridge, for example, that is capable of selectively reversing the polarity of the voltage applied to electrolysis cell 18 as a function of a control signal generated by the control circuit. For example, control circuit 30 can be configured to alternate polarity in a predetermined pattern, such as every 5 seconds with a 50% duty cycle. In another example, described above, control circuit 30 is configured to apply a voltage to the cell with primarily a first polarity and periodically reverse the polarity for only very brief periods of time.

In the context of a hand-held spray bottle, it is inconvenient to carry large batteries. Therefore, the available power to the pump and cell is somewhat limited. In one example, the driving voltage for the cell is in the range of about 18 Volts to about 28 Volts. But since typical flow rates through the spray bottle and electrolysis cell are fairly low, only relatively small currents are necessary to effectively activate the liquid passing through the cell. With low flow rates, the residence time within the cell is relatively large. The longer the liquid resides in the cell while the cell is energized, the greater the electrochemical activation (within practical limits). This allows the spray bottle, for example, to employ smaller capacity batteries and a DC-to-DC converter, which steps the voltage up to the desired output voltage at a low current.

In one particular example in which the spray bottle carries four AA batteries, the batteries may have an output voltage in a range of about 3 Volts to about 9 Volts, or for example. For example, each AA battery may have, for example, a nominal output voltage of 1.5 Volts at about 500 milliampere-hours to about 3 ampere-hours. If the batteries are connected in series, then the nominal output voltage would be about 6V with a capacity of about 500 milliampere-hours to about 3 ampere-hours. This voltage can be stepped up to the range of 18 Volts to 28 Volts, or in a range of 18 Volts to 38 Volts, for example, through the DC-to-DC converter. Thus, the desired electrode voltage can be achieved at a sufficient current.

In another particular example, the spray bottle carries ten nickel-metal hydride batteries, each having a nominal output voltage of about 1.2 Volts. The batteries are connected in series, so the nominal output voltage is about 10 Volts to about 13.8 Volts with a capacity of about 1800 milliampere-hours, for example. This voltage is stepped up/down to a range of 8 Volts to at least 28 Volts or to a range of about 8 Volts to about 38 Volts, for example, through the DC-to-DC converter. Thus, the desired electrode voltage can be achieved at a sufficient current. It will be appreciated that as the sizes of batteries decrease, even smaller battery sizes, numbers, combinations, or capacities thereof or of other related electrical devices such as converters, etc. may be utilized in alternate embodiments.

The ability to produce a large voltage and a suitable current through the cell can be beneficial for applications in which regular tap water is fed through the cell to be converted into a liquid having enhanced cleaning and/or sanitizing properties. Regular tap water has a relatively low electrical conductivity between the electrodes of the cell.

Examples of suitable DC-to-DC converters include the Series A/SM surface mount converter from PICO Electronics, Inc. of Pelham, N.Y., U.S.A. and the NCP3064 1.5 A Step-Up/Down/Inverting Switching regulator from ON Semiconductor of Phoenix, Ariz., U.S.A, connected in a boost application.

In one example, the control circuit controls the DC-to-DC converter based on a sensed current drawn from the electrolysis cell so that the DC-to-DC converter outputs a voltage that is controlled to achieve a current draw through the cell that is within a predetermined current range. For example, the target current draw is about 400 milliamperes in one specific example. In another example, the target current is 350 milliamperes. Other currents and ranges can be used in alternative embodiments. The desired current draw may depend on the geometry of the electrolysis cell, the properties of the liquid being treated and the desired properties of the resulting electrochemical reaction.

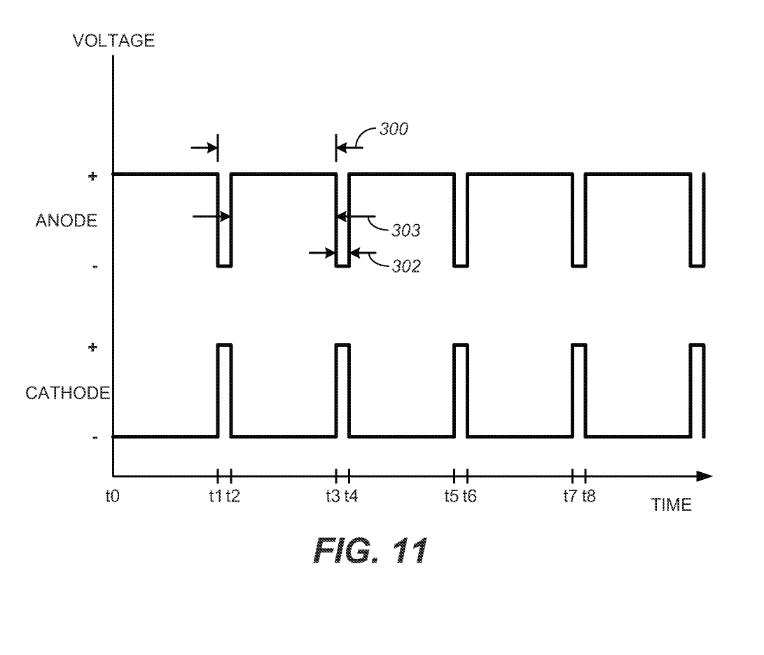

A block diagram illustrating a particular example of the control circuit 30 is shown in FIG. 12. Although the control circuit shown in FIG. 12 is configured to control various components of a spray bottle such as that shown in FIGS. 10A-10C, the control circuit can be used as is or modified as desired to control similar elements on any other apparatus according to alternative embodiments of the present disclosure.

The main components of control circuit 30 include a microcontroller 1000, a DC-to-DC converter 1004, and an output driver circuit 1006.

Power to the various components is supplied by a battery pack 542 carried by the bottle, as shown in FIG. 10B, for example. In a specific example, battery pack 542 includes ten nickel-metal hydride batteries, each having a nominal output voltage of about 1.2 Volts. The batteries are connected in series, so the nominal output voltage is about 10V to 12.5V with a capacity of about 1800 milliampere-hours. Hand trigger 570,572 (shown in FIGS. 10A-10C, for example) selectively applies the 12-volt output voltage from battery pack 542 to voltage regulator 1003 and to DC-to-DC converter 1004. Any suitable voltage regulator can be used, such as an LM7805 regulator from Fairchild Semiconductor Corporation. In a particular example, voltage regulator 1003 provides a 5 Volt output voltage for powering the various electrical components within the control circuit.

DC-to-DC converter 1004 generates an output voltage to be applied across the electrodes of electrolysis cell 552. The converter is controlled by microcontroller 1000 to step the drive voltage up or down in order to achieve a desired current draw through the electrolysis cell. In a particular example, converter 1004 steps the voltage up or down between a range of 8 Volts to 28 Volts (or greater) to achieve a current draw through electrolysis cell 552 of about 400 milliamps, as pump 550 pumps water from container 510, through cell 552 and out nozzle 508 (FIGS. 10A-10C). The required voltage depends in part on the conductivity of the water between the cell's electrodes.

In a particular example, DC-to-DC converter 1004 includes a Series A/SM surface mount converter from PICO Electronics, Inc. of Pelham, N.Y., U.S.A. In another example, converter 1004 includes an NCP3064 1.5 A Step-Up/Down/Inverting Switching regulator from ON Semiconductor of Phoenix, Ariz., U.S.A, connected in a boost application. Other circuits and/or arrangements can be used in alternative embodiments.

Output driver circuit 1006 selectively reverses the polarity of the driving voltage applied to electrolysis cell 552 as a function of a control signal generated by microcontroller 1000. For example, microcontroller 1000 can be configured to alternate polarity in a predetermined pattern, such that shown and/or described with reference to FIG. 11. Output driver 1006 can also provide an output voltage to pump 550. Alternatively, for example, pump 550 can receive its output voltage directly from the output of trigger switch 570, 572.

In a particular example, output driver circuit 1006 includes a DRV 8800 full bridge motor driver circuit available from Texas Instruments Corporation of Dallas, Tex., U.S.A. Other circuits and/or arrangements can be used in alternative embodiments. The driver circuit 1006 has an H-switch inverter that drives the output voltage to electrolysis cell 552 according to the voltage pattern controlled by the microcontroller. The H-switch also has a current sense output that can be used by the microcontroller to sense the current drawn by cell 552. Sense resistor R_{SENSE} develops a voltage that is representative of the sensed current and is applied as a feedback voltage to microcontroller 1000. Microcontroller 1000 monitors the feedback voltage and controls converter 1004 to output a suitable drive voltage to maintain a desired current draw.

Microcontroller 1000 also monitors the feedback voltage to verify that electrolysis cell 552 and/or pump 550 is operating properly. As discussed above, microcontroller 1000 can operate LEDs 594 and 596 as a function of the current levels sensed by output driver circuit 1006. For example, microcontroller 1000 can turn off (or alternatively, turn on) one or both of the sets of LEDs 594 and 596 as a function of whether the current level sensed is above or below a threshold level or within a range.

Output driver circuit 1006 can also deliver a drive voltage to pump 550 under the control of microcontroller 1000, which turns the pump on and off upon actuation of user trigger switch 570, 572. For example, output driver circuit 1006 can selectively apply the 12-volt battery voltage and/or the return voltage to pump 550 through a switch, such as a power MOSFET. In one particular example, the return voltage is selectively gated with an IRF7603pbF power MOSFET available from International Rectifier of El Segundo, Calif.

Microcontroller 1000 can include any suitable controller, processor, and/or circuitry. In a particular embodiment, it includes an MC9S08SH4CTG-ND Microcontroller available from Digi-Key Corporation of Thief River Falls, Minn., U.S.A.

In the example shown in FIG. 12, the illumination control portion of the circuit includes output resistors R1 and R2 and a first, “red” LED control leg formed by pull-up resistor R3, red LED diodes D1-D4, and pull-down transistor Q1. Microcontroller 1000 has a first control output, which selectively turns on and off red LEDs D1-D4 by turning on and off transistor Q1. The illumination control portion of the circuit further a second, “green” LED control leg formed by pull-up resistor R4, green LED diodes D5-D8, and pull-down transistor Q2. Microcontroller 1000 has a second control output, which selectively turns on and off green LEDs D5-D8 by turning on and off transistor Q2.

The control circuit further includes a control header 1002, which provides an input for programming microcontroller 1000.

In one particular example, the elements 1000, 1002, 1003, 1004, 1006, R1-R4, D1-D8 and Q1-Q2 reside on circuit board 540, shown in FIG. 10B.

In addition, the control circuit shown in FIG. 12 can include a charging circuit (not shown) for charging the batteries within battery pack 542 with energy received through the power jack 523 shown in FIGS. 10B and 10C.

One or more of the control functions described herein can be implemented in hardware, software, firmware, etc., or a combination thereof. Such software, firmware, etc. is stored on a computer-readable medium, such as a memory device. Any computer-readable memory device can be used, such as a disc drive, a solid state drive, CD-ROM, DVD, flash memory, RAM, ROM, a set of registers on an integrated circuit, etc.

### 6.2.3 Driving Voltage for Electroporation Electrode Example

The electroporation electrode 35 (such as adapter 240 in FIG. 6) can be driven with any suitable driving voltage pattern to achieve the desired microorganism de-activation level. The electrical characteristics of the driving voltage pattern will be based on the design of the apparatus and the method of application of the liquid to the microorganism.

In one example of a spray bottle disclosed herein, the driving voltage applied to the electrode has a frequency in the range of 25 kilohertz to 800 kilohertz and a voltage of 50 Volts to 1000 Volts root-mean-square (rms). However, the applied current can be very low, such as but not limited to the order of 0.15 milliamps. The voltage pattern can be a DC pattern, and AC pattern or a combination of both. The voltage waveform can be any suitable type such as square, sinusoidal, triangular, sawtooth, and/or arbitrary (from arbitrary pattern generator). In one example, the waveform sequentially changes between various waveforms. The positive (or alternatively negative) side of the voltage potential is applied to the electrode, and the potential of the surface (or volume of space) being treated serves as the circuit ground (such as Earth ground), for example. In addition, the waveforms and voltage levels may affect different microorganisms differently. So these parameters can be modified to enhance killing of particular microorganisms or can be varied during application to treat effectively a variety of different organisms.

Examples of suitable voltages applied to the electroporation electrode include but are not limited to AC voltages in a range of 50 Vrms to 1000 Vrms, 500 Vrms to 700 Vrms, or 550 Vrms to 650 Vrms. One particular embodiment applies an voltage of about 600 Vrms to the electroporation electrode.

Examples of frequencies for the voltage that is applied to the electroporation electrode include but are not limited to those frequencies within a range of 20 KHz to 100 KHz, 25 KHz to 50 KHz, 30 KHz to 60 KHz, or about 28 Khz to about 40 KHz. One particular embodiment applies the voltage at about 30 KHz to the electroporation electrode.

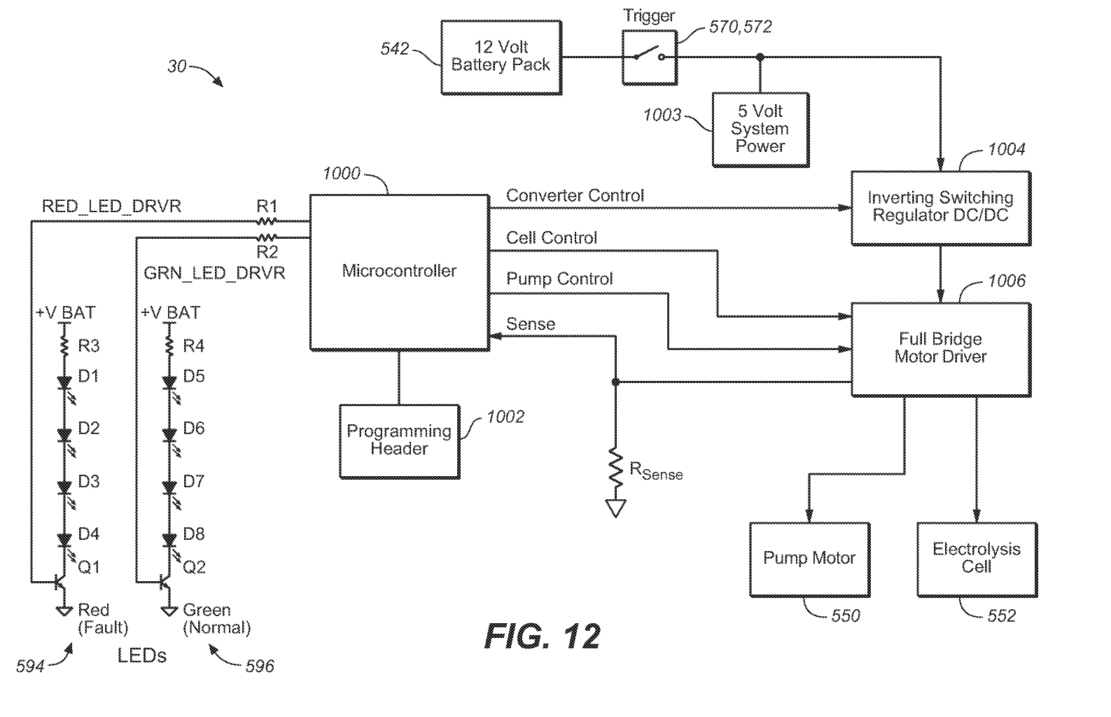

FIG. 13A is a waveform diagram illustrating the voltage pattern applied to electroporation electrode 35 in one particular example. In this example, the shape of the waveform is a combination of a sine wave and a square wave. However, the waveform can have other shapes, such as a sine wave, a square wave, or other waveform. The applied voltage has an AC voltage of 600 Volts rms (about 1000V to 1200 Volts peak-to-peak) when liquid is flowing through adapter 240 of the electrode and has a frequency of about 30 KHz. In this example, the frequency remains substantially constant as the apparatus (e.g., spray bottle) dispenses electrochemically-activated liquid to the surface being treated. In another example, the frequency is maintained in a range of about 41 KHz-46 KHz.

In another example, the frequency varies over a predefined range while the apparatus (e.g., spray bottle) dispenses electrochemically-activated liquid to the surface being treated. For example, the control circuit that drives electroporation electrode 35 can sweep the frequency within a range between a lower frequency limit and an upper frequency limit, such as between 20 KHz and 100 KHz, between 25 KHz and 50 KHz, and between 30 KHz and 60 KHz.

FIG. 13B is a waveform diagram illustrating the frequency with respect to time of the voltage applied to electroporation electrode 35 in another particular example. In this example, the frequency ramps, with a triangular waveform, from the low frequency limit to the high frequency limit and then back down to the low frequency limit over a period of about 1 second, for example. In another example, the control circuit ramps the frequency from the from the low frequency limit to the high frequency limit (and/or from the high frequency limit to the low frequency limit) over a time period of 0.1 second to 10 seconds. Other ramp frequency ranges can also be used, and the respective ramp-up and ramp-down periods can be the same or different from one another. Since different microorganisms might be susceptible to irreversible electroporation at different frequencies, the killing effect of the applied voltage is swept between different frequencies to potentially increase effectiveness on different microorganisms. For example, sweeping the frequency might be effective in applying the potential at different resonant frequencies of different microorganisms.

In the example shown in FIG. 13C, the frequency is swept between 30 KHz and 60 KHz with a sawtooth waveform. Other waveforms can also be used.

### 6.2.4 Control Circuit for Electroporation Electrode Example

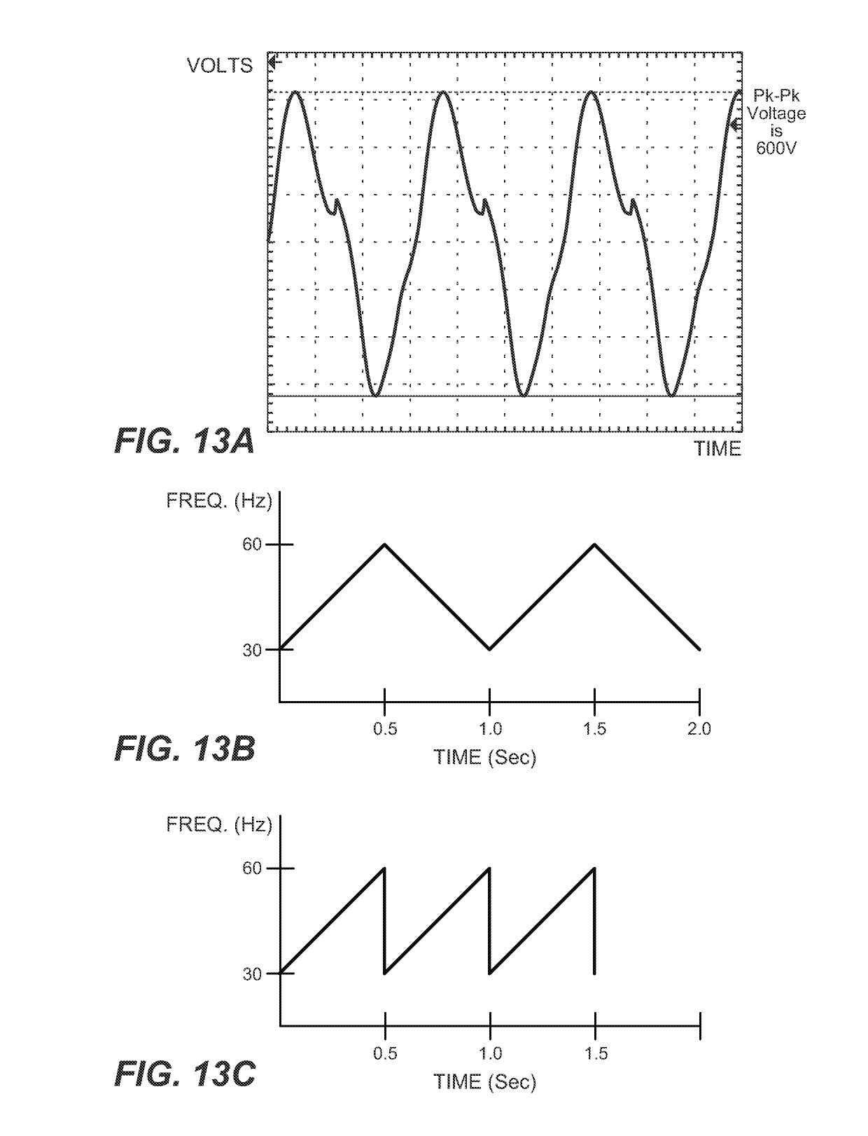

FIG. 14 is a block diagram illustrating an example of a control circuit 1100 for providing a voltage potential to electroporation electrode 35. Circuit 1100 includes a voltage input connector 1102, a voltage regulator 1104, a tri-color LED 1106, microcontroller 1108, switching power controller 1110, H-bridge circuits 1112 and 1114, transformer 1116, voltage divider 1118, sense resistor 1120 and output connector 1122.

Input connector 1102 receives the 12-Volt battery supply voltage from the main circuit board, shown in FIG. 12 for example, and supplies the voltage to voltage regulator 1104, switching power controller 1110 and H-bridge circuits 1112 and 1114. In a particular example, voltage regulator 1104 provides a 5 Volt output voltage for powering the various electrical components within the control circuit 1100, such as microcontroller 1108, LED 1106 and Switching power controller 1110. Any suitable voltage regulator can be used, such as an LM7805 regulator from Fairchild Semiconductor Corporation.

In this embodiment microcontroller 1108 has three main functions; providing a clock signal (SYNC) and an enable signal (ENABLE) to switching power regulator 1110, monitoring for fault conditions, and providing a user an indication of a fault condition through LED 1106. In one example, microcontroller 1108 comprises an ATtiny24 QPN Microcontroller available from ATMEL Corporation. Other controllers can be used in alternative embodiments.

The clock signal SYNC provides a reference frequency for switching power controller 1110. Enable signal ENABLE, when active, enables (or turns on) switching power controller 1110. Normally, microcontroller 1108 sets ENABLE to an active state and monitors the FAULT signal for a fault condition. When no fault condition is present, microcontroller 1108 selectively turns on one or more colors of the tri-color LED 1106. In one example, LED 1106 is a tri-color red, green, blue LED. However, multiple, separate LEDs can be used in alternative embodiments. Further, other types of indicators can be used in addition or in replace of LED 1106, such as any visual, audible or tactile indicator. In the present example, microcontroller 1108 illuminates a blue LED by pulling the respective cathode low when no fault condition is present.

When controller 1110 indicates a fault condition by activating the signal FAULT, microcontroller 1108, selectively pulses the ENABLE signal to an inactive state and then returns it to the active state to reset switching power controller 1110. If the fault condition clears, microcontroller continues to illuminate the blue LED. If the fault condition remains active, then microcontroller turns off the blue LED and illuminates a red LED. The green LED is not used, but could be used in alternative embodiments. Other user indication patterns can be used in alternative embodiments.

In one example, switching power controller 1110 includes a TPS68000 CCFL Phase Shift Full Bridge CCFL Controller available from Texas Instruments. However, other types of controllers can be used in alternative embodiments.

Based on the SYNC signal, switching power controller 1110 provides gate control signals to the gates of switching transistors within the H-bridge circuits 1112 and 1114. In one example, H-bridge circuits 1112 and 1114 each include an FDC6561AN Dual N-Channel Logic Level MOSFET (although other circuits can be used), which are connected together to form an H-bridge inverter that drives the primary side of transformer 1116 with the desired voltage pattern, such as that shown in FIG. 13. Transformer 1116 has a 1:100 turn ratio, which steps the drive voltage from about 10V-13V peak-to-peak up to about 1000V to 1300 V peak-to-peak (about 600 V rms), for example, when liquid is being dispensed from the apparatus. The output drive voltage is applied to the electroporation electrode 35 through output connector 1122.

Voltage divider 1118 comprises a pair of capacitors that are connected in series between the primary side of the transformer and ground to develop a voltage that is feed back to switching power controller 1110 and represents the voltage developed on the secondary side of the transformer. This voltage level is used to detect an over-voltage condition. If the feedback voltage exceeds a given threshold, switching power controller 1110 will activate fault signal FAULT.

Sense resistor 1120 is connected between the primary side of the transformer and ground to develop a further feedback voltage that is feed back to switching power controller 1110 and represents the current flowing through the secondary side of the transformer. This voltage level is used to detect an over-current condition. If the feedback voltage exceeds a given threshold, switching power controller 1110 will activate fault signal FAULT, indicating a fault in the transformer.

In addition, the source of the bottom transistor in one leg of the H-bridge is fed back to switching power controller 1110, as shown by arrow 1124. This feedback line can be monitored to measure the current in the primary side of the transformer, which can represent the current delivered to the load through electroporation electrode 35. Again, this current can be compared against a high and/or a low threshold level. The result of the comparison can be used to set the state of fault signal FAULT.

### 7. Other Exemplary Apparatus for Delivering Electrical Charge through an Output Liquid

The features and methods described herein, such as those of the electrolysis cell and/or the electroporation electrode, can be used in a variety of different apparatus, for example, including on a spray bottle, a mobile surface cleaner, and/or a free-standing or wall-mount platform.

For example, they can be implemented onboard (or off-board) a mobile surface cleaner, such as a mobile hard floor surface cleaner, a mobile soft floor surface cleaner or a mobile surface cleaner that is adapted to clean both hard and soft floors or other surfaces, an all-surface cleaner, truck-mounted sprayer, high-pressure bathroom sprayer, toilets and urinals, for example.

### 7.1 Mobile surface Cleaner Example

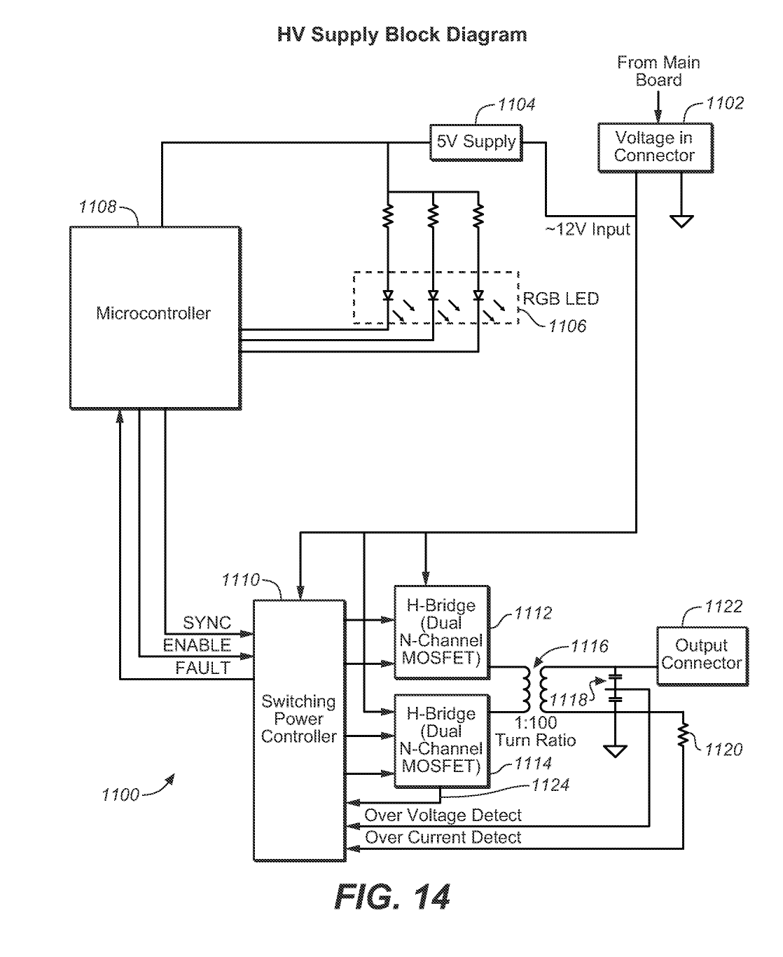

FIG. 15 illustrates an example of a mobile hard and/or soft floor surface cleaner 1200 disclosed in Field et al. U.S. Publication No. 2007/0186368 A1, which can be modified to implement one or more of the above-described features and/or methods. FIG. 15 is a perspective view of cleaner 1200 having its lid in an open position.

In this example, cleaner 1200 is a walk-behind cleaner used to clean hard floor surfaces, such as concrete, tile, vinyl, terrazzo, etc. in other examples, cleaner 1200 can be configured as a ride-on, attachable, or towed-behind cleaner for performing a cleaning and/or sanitizing operation as described herein. In a further example, cleaner 1200 can be adapted to clean soft floors, such as carpet, or both hard and soft floors in further embodiments. Cleaner 1200 may include electrical motors powered through an on-board power source, such as batteries, or through an electrical cord. Alternatively, for example, an internal combustion engine system could be used either alone, or in combination with, the electric motors.

Cleaner 1200 generally includes a base 1202 and a lid 1204, which is attached along one side of the base 1202 by hinges (not shown) so that lid 1204 can be pivoted up to provide access to the interior of base 1202. Base 1202 includes a tank 1206 for containing a liquid or a primary cleaning and/or sanitizing liquid component (such as regular tap water) to be treated and applied to the floor surface during cleaning/sanitizing operations. Alternatively, for example, the liquid can be treated onboard or offboard cleaner 1200 prior to containment in tank 1206. In addition, cleaner 1200 includes an electrolysis cell 1208, which treats the liquid prior to the liquid being applied to the floor being cleaned. Electrolysis cell 1208 can include, for example, one or more electrolysis cells (in parallel or in series with one another) similar to the one shown and discussed above with reference to FIG. 5 or for example, one or more of the electrolysis cells disclosed in Field et al. U.S. Publication No. 2007/0186368 A1, including but not limited to the electrolysis cells (e.g., functional generators) disclosed in FIGS. 8A and 8B. For example, the electrolysis cell shown in FIGS. 8A and 8B can include an unmodified or modified Emco Tech “JP102” cell found within the JP2000 ALKABLUE LX, which is commercially available from Emco Tech Co., LTD, of Yeupdong, Goyang-City, Kyungki-Do, South Korea. This particular cell has a DC range of 27 Volts, a pH range of about 10 to about 5.0, a cell size of 62 mm by 109 mm by 0.5 mm, and five electrode plates. In an example modified version, the JP102 cell is modified to remove a valve mechanism that is supplied with the JP102 cell (and selectively routes the anolyte and catholyte to separate, respective outlets) such that produced anolyte and catholyte mix together to form blended anolyte and catholyte EA water, for example, which is directed to an outlet of the cell. Other types of electrolysis cells can also be used, which can have various different specifications.

The treated liquid can be applied to the floor directly and/or through a cleaning head 1210, for example. The treated liquid that is applied to the floor can include an anolyte EA liquid stream, a catholyte EA liquid stream, both and anolyte and catholyte EA liquid streams and/or a combined anolyte and catholyte EA liquid stream, as described above with reference to FIG. 2, for example. The cell 1208 can include an ion selective membrane or be configured without an ion selective membrane.

In one example, to enhance the electroporation/electrohydraulic shock properties of the output liquid, the liquid flow path is applied directly to the floor to avoid disruption of the electrical conduction path between the electrolysis cell and the floor that is formed by the liquid flow path. The liquid can be applied in any form, such as a stream, an aerosolizing mist, and/or a spray.

In one example, (with or without electrolysis cell 1208), cleaner 1200 is further modified to include a further electrical conductor or lead, for example an electroporation electrode (such as electrode 35 shown in FIGS. 1 and 6), at any location along, or in appropriate relation to, the liquid flow path. This electrode can become electrically connected to the floor being treated via liquid flowing through the flow path. In one example, the electrode is located at a position very near the point at which the liquid is output from the cleaner, such as along a dispensing tube 1212 near cleaning head 1210. Alternatively or in addition, the electrode can be located near a spray nozzle that dispenses an output spray or stream ahead of cleaning head 1210, onto or through the cleaning head, or behind the cleaning head, for example, with respect to a direction of travel of cleaner 1200. The electrode can have any suitable construction, shape or material, for example.

If desired, further structures of one or more particular non-limiting examples of the mobile cleaner 1200 are shown and described in more detail in Field et al U.S. Publication No. 2007/018368, which is incorporated by reference in its entirety above. These structures can be used in any of the embodiments disclosed herein and modifications thereof. The details of at least one particular example are described in FIGS. 10A-10C and 11, for example, of U.S. Publication No. 2007/018368.

Field et al. U.S. Publication No. 2007/0186368 A1 also discloses other structures on which the various structural elements and processes disclosed herein can be utilized either separately or together. For example, Field et al. disclose a wall mount platform for generating anolyte and catholyte EA liquid. Any of these apparatus can be configured according to disclosure herein in order to provide an electric field to a surface being treated while the surface is being cleaned and/or sanitized.

In another embodiment, the mobile cleaner 1200 does not include an electrolysis cell but e.g. in addition or instead includes a detergent dispenser, which dispenses detergent with source liquid to the surface being cleaned. The detergent in combination with a mechanical action of the cleaning head can suspend microorganisms in liquid on the surface so that they may be more easily electroporated by an electric field applied by an electroporation electrode as disclosed herein.

### 7.2 All Surface Cleaner Example

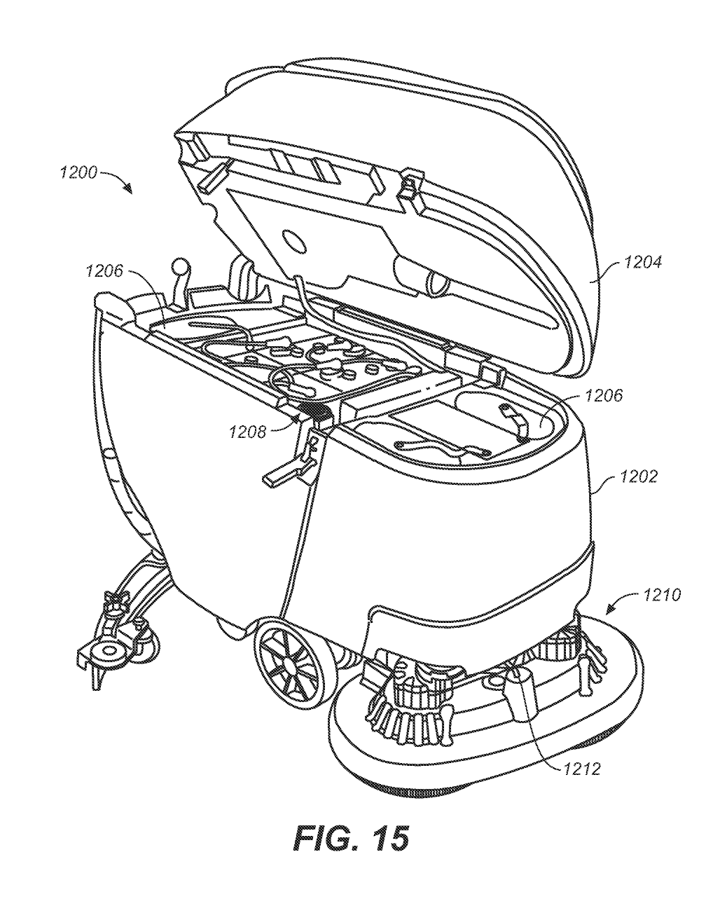

FIG. 16 is a perspective view of an example of an all surface cleaning assembly 1300, which is described in more detail in U.S. Pat. No. 6,425,958, which is incorporated herein by reference in its entirety. The cleaning assembly 1300 is modified to include a liquid distribution path with one or more electrolysis cells and/or one or more electroporation electrodes described herein such as but not limited to those shown or described with reference to FIGS. 1-3 and 5-6, for example, or any of the other embodiments disclosed herein.

Cleaning assembly 1300 can be constructed to deliver and optionally recover one or more of the following liquids, for example, to and from the floor being cleaned: anolyte EA water, catholyte EA water, blended anolyte and catholyte EA water, or other electrically-charged liquids. For example, liquid other than or in addition to water can be used.

Cleaning assembly 1300 can be used to clean hard surfaces in restrooms or any other room having at least one hard surface, for example. Cleaning assembly 1300 includes the cleaning device and the accessories used with the cleaning device for cleaning the surfaces, as described in U.S. Pat. No. 6,425,958. Cleaning assembly 1300 includes a housing 1301, a handle 1302, wheels 1303, a drain hose 1304 and various accessories. The accessories can include a floor brush 1305 having a telescoping and extending handle 1306, a first piece 1308A and a second piece 1308B of a two piece double bend wand, a spray gun 1310 and various additional accessories not shown in FIG. 16, including a vacuum hose, a blower hose, a sprayer hose, a blower hose nozzle, a squeegee floor tool attachment, a gulper tool, and a tank fill hose (which can be coupled to ports on assembly 1300). The assembly has a housing that carries a tank or removable liquid container and a recovery tank or removable recovery liquid container. The cleaning assembly 1300 is used to clean surfaces by spraying the cleaning liquid through a sprayer hose and onto the surfaces. The blower hose is then used to blow dry the surfaces and to blow the fluid on the surfaces in a predetermined direction. The vacuum hose is used to suction the fluid off of the surfaces and into the recovery tank within cleaning device 1300, thereby cleaning the surfaces. The vacuum hose, blower hose, sprayer hose and other accessories used with cleaning assembly 1300 can be carried with the cleaning device 1300 for easy transportation. Spray gun 1310 is attached to a liquid outlet 1312 of cleaner 1300 through a hose 1314.

An electroporation electrode can be located at any location along, or in appropriate relation to, the liquid flow path, which for example can become electrically connected to the surface being treated by via liquid flowing through the flow path. For example, the electrode can be located at the spray head of spray gun 1310, along the spray hose and/or at any suitable location on the assembly, such as near the outlet 1303. The cleaning device also carries the control circuits for the electrolysis cell and the electroporation electrode.

In another example, a wall-mounted platform supports an electrolysis cell and/or electroporation electrode along the liquid flow path from an inlet of the platform to an outlet of the platform. In this embodiment, a hose or other liquid dispenser, for example, would carry the liquid to the point of application to the surface being treated.

### 10. Flat Mop Example

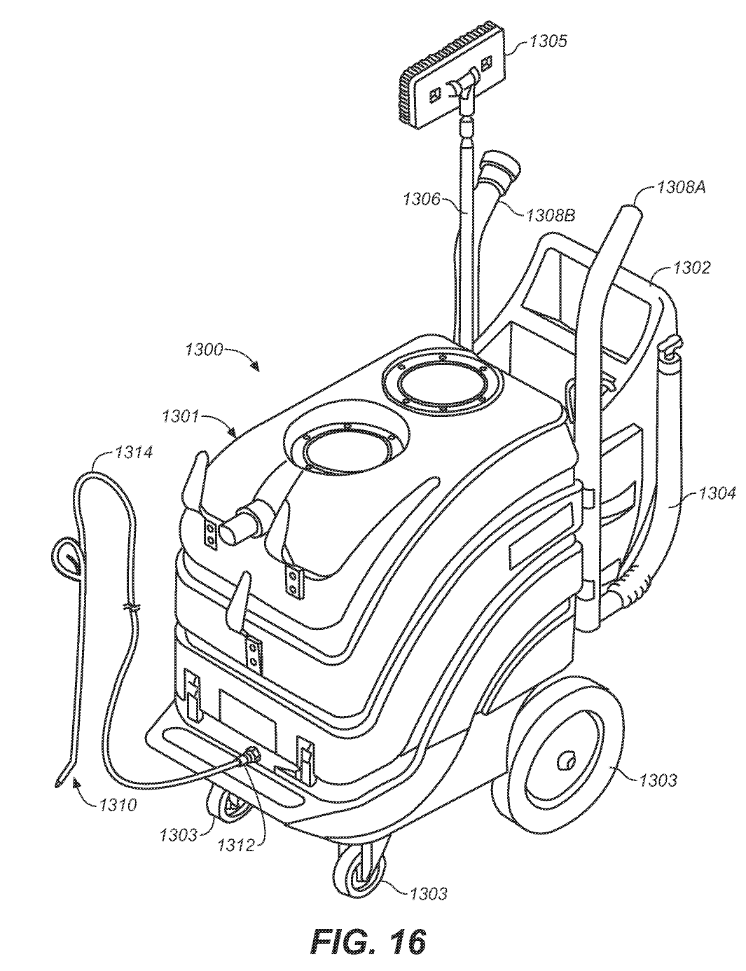

FIG. 17 is a diagram illustrating an example of a flat mop embodiment, which includes at least one electrolysis cell and/or at least one electrical conductor, lead and/or electromagnetic component to impart, induce or otherwise cause an electrical potential in the liquid output spray, for example an electroporation electrode, such as those described herein in the present disclosure.

In this example, flat mop 1400 includes a stiff backing 1402, which can be fitted with a cleaning pad 1404, such as a micro-fiber pad or cloth. A handle 1405 extends from the backing 1402 and carries a reservoir 1406 and a compartment 1408. Reservoir 1406 is adapted to hold a source liquid, such as regular tap water, and can be filled through a fill port 1410. Reservoir 1406 supplies the source liquid to compartment 1408, which can include, for example, a pump, at least one electrolysis cell and/or at least one electroporation electrode, and respective and/or combined control electronics.

On one particular example, compartment 1408 includes the component parts of the hand-held spray device shown and described with reference to FIGS. 5, 6, 10A-10C and 11-14 (or any of the other examples or embodiments described herein, for example). Compartment 1408 includes a spray nozzle 1412, similar to spray nozzle 508 in FIGS. 10A-10C. An electroporation electrode is coupled at any suitable location in the liquid flow path from reservoir 1406 to nozzle 1412, such as at a location close to the nozzle. Nozzle sprays or otherwise dispenses an output spray or stream 1414 toward the surface being cleaned and/or sanitized, wherein the dispensed liquid can be electrochemically activated as described herein, for example. In addition, or in the alternative, the electroporation electrode applied an electric field through the output spray 1414 to the surface, which for example, is sufficient to cause irreversible electroporation of microorganisms on the surface.

Handle 1405 includes a switch 1416, which is operable by a user similar to trigger 570 in FIGS. 10A-10C, to selectively energize the pump, electrolysis cell, and electroporation electrode. For example, switch 1416 can include a momentary or non-momentary push button or trigger.

### 11. Stationary (or Portable) Device Example

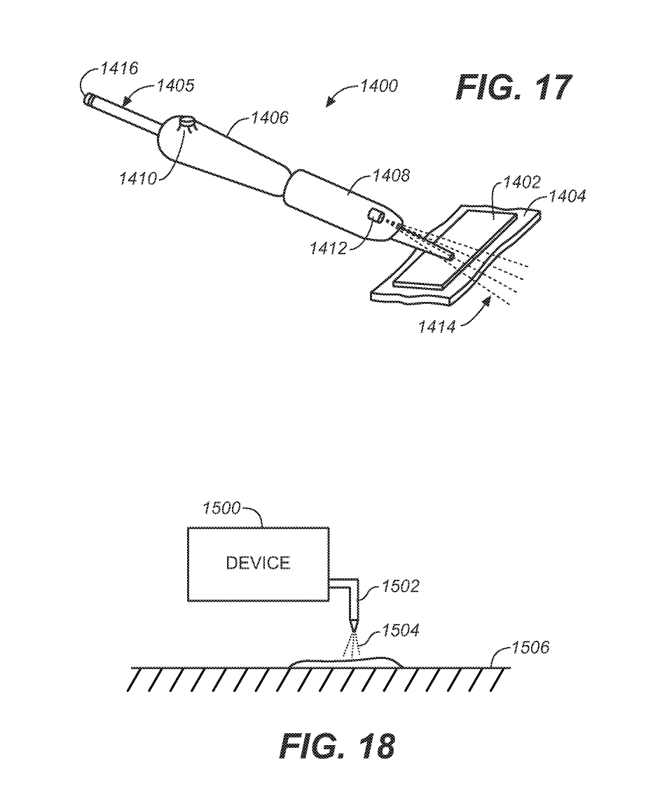

FIG. 18 is a diagram illustrating an example device 1500, which can be stationary or movable relative to a surface 1502. In one example, device 1500 includes the component parts of the hand-held spray device shown and described with reference to FIGS. 5, 6, 10A-10C and 11-14 (or any of the other examples or embodiments described herein, for example), which can include, for example, a pump, at least one electrolysis cell and/or at least one electroporation electrode, and respective and/or combined control electronics. Device 1500 includes an outlet 1502, which sprays or otherwise dispenses an output spray or stream 1504 to the surface 1506 and/or item being cleaned and/or sanitized. Surface 1506 can be stationary and/or movable relative to device 1500. The arrangement can be adapted to clean and/or sanitize the surface 1506 itself and/or one or more items carried by the surface. For example, the surface can include a table surface or a conveyor carrying product. The dispensed liquid 1504 can be electrochemically activated as described herein. In addition, or in the alternative, an electroporation electrode can be coupled at any suitable location in the liquid flow path, such as at a location close to the outlet 1502, wherein the electroporation electrode applies an electric field through the dispensed liquid 1504 to the surface or item, which for example, is sufficient to cause irreversible electroporation of microorganisms on the surface or item.

### 12. Further System Example

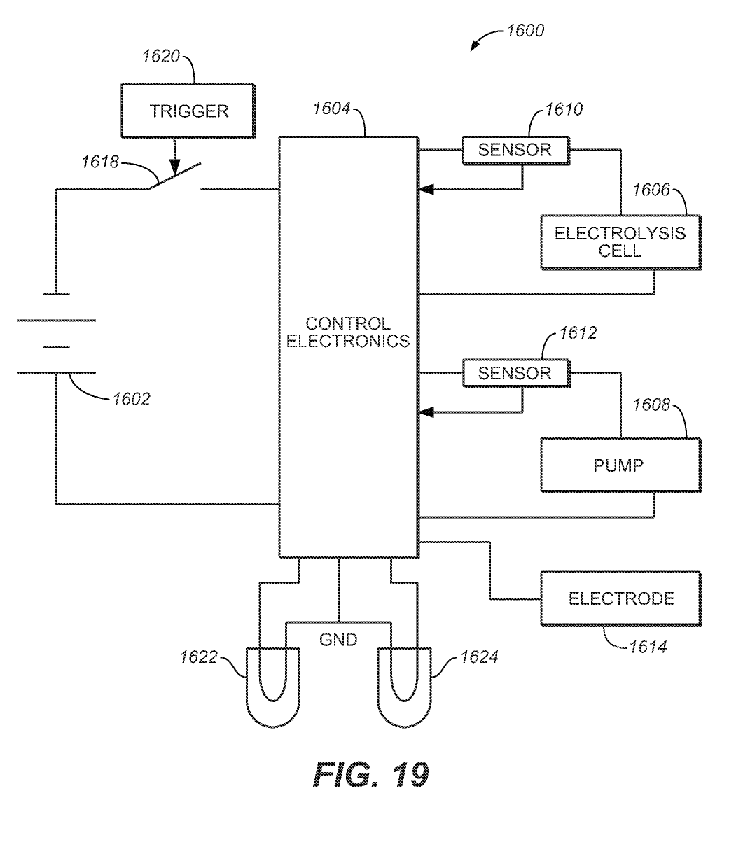

FIG. 19 is a diagram, which illustrates a system 1600 according to an example embodiment of the disclosure, which can be incorporated into any of the embodiments disclosed herein, for example. System 1600 includes power supply (such as a battery) 1602, control electronics 1604, electrolysis cell 1606, pump 1608, current sensors 1610 and 1612, an electroporation electrode 1614, switch 1618 and trigger 1620. For simplicity, the liquid inputs and outputs of electrolysis cell 1604 are not shown in FIG. 19. All elements of system 1600 can be powered by the same power supply 1602 or by two or more separate power supplies, for example.

Control electronics 1604 are coupled to control the operating state of electrolysis cell 1606, pump 1608 and electrode based on the present operating mode of system 1600 and user control inputs, such as trigger 1620. In this example, switch 1618 is coupled in series between power supply 1602 and control electronics 1604 and serves to couple and decouple power supply 1602 to and from power inputs of control electronics 1604 depending on the state of trigger 1620. In one embodiment, switch 1618 includes a momentary, normally-open switch that closes when trigger 1620 is depressed and opens when trigger 1620 is released.

In an alternative example, switch 1618 is configured as an on/off toggle switch, for example, that is actuated separately from trigger 1620. Trigger 1620 actuates a second switch that is coupled to an enable input of control electronics 1604. The same switch 1618 can be used to control power to the various devices 1606, 1608 and 1614 or separate switches can be used. Also, the same or separate power supplies and/or sources can be used to power the various devices 1606, 1608 and 1614. In addition, the same or separate control circuits can be used to control the voltages applies the electrolysis cell 1606, pump 1608 and electrode 1614. Other configurations can also be used.

In one example, when trigger 1620 is depressed, control electronics 1604 is enabled and generates appropriate voltage outputs for driving electrolysis cell 1606, pump 1608 and electrode 1614. For example, control electronics 1604 can produce a first voltage pattern for driving the electrolysis cell 1606, a second voltage pattern for driving pump 1608, and a third voltage pattern for electrode 1614, such as those patterns described herein. When trigger 1620 is released, control electronics is powered off and/or otherwise disabled from producing the output voltages to cell 1606 and pump 1608.

Current sensors 1610 and 1612 are coupled in electrical series with electrolysis cell 1606 and pump 1608, respectively, and each provide a signal to control electronics 1604 that is representative of the respective electrical current drawn through cell 1606 or pump 1606. For example, these signals can be analog or digital signals. Control electronics 1604 compares the sensor outputs to predetermined threshold current levels or ranges and then operates indicators 1614 and 1616 as a function of one or both of the comparisons. The threshold current levels or ranges can be selected to represent predetermined power consumption levels, for example. The bottle can also be provided with a visually perceptible indicator(s), such as one or more LEDs 1622 and 1624, which can illuminate in different colors or illumination patterns to indicate different operating states, for example.

In addition, a switch can be placed in series with electrode 1614 (or as a control input to control electronics 404) to selectively disable electrode 1614 when enhanced sanitization properties are not needed. Disabling electrode 1614 may lengthen the battery life or charge state of power source 1602, when a small power supply is used.

### 13. Test Results

### Examples

The present disclosure is more particularly described in the following examples that are intended as illustrations only, since numerous modifications and variations within the scope of the present disclosure will be apparent to those skilled in the art. Unless otherwise noted, all parts, percentages, and ratios reported in the following examples are on a weight basis, and component weight percents are based on the entire weight of the membrane, excluding any reinforcement matrix used. All reagents used in the examples were obtained, or are available, from the chemical suppliers described below, from general chemical suppliers such as Sigma-Aldrich Company, Saint Louis, Mo., or may be synthesized by conventional techniques.

### 13.1 Example 1

### Electric Field Measurements

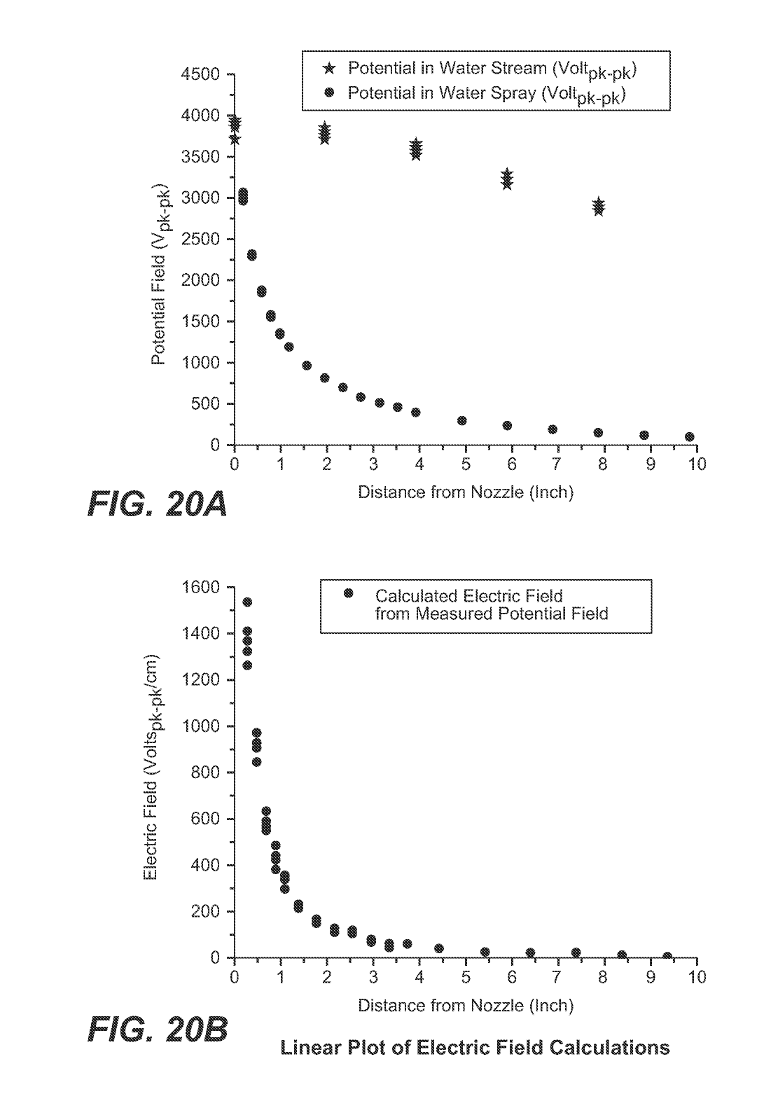

Electric field measurements were conducted on a spray bottle of Example 1, which was based on the embodiments shown and described with reference to FIGS. 5, 6, 10A-10C and 11-14 above. Five measurements were made at each linear position from the spray nozzle of Example 1 along the spray axis. The average results are plotted in FIG. 20. For comparison purposes with the water spray results, a length of rubber hose was attached to the outlet of the spray bottle and the electrical potential relative to ground was measured across a 1 MegaOhm load at the end of this water stream. The rubber hose was then shortened and the measurement repeated until the measurement position was near the sprayer nozzle. The water stream forms a true electrical conductive path, and four measurements were taken at each position.

FIG. 20A plots the potential field (Vpeak-peak) as a function of distance from the nozzle (inches). FIG. 20B plots the electric field (Volts peak-peak/cm) linearly as a function of distance from the nozzle (inches), which was calculated from the potential field data using two-point numerical differentiation.

As seen in FIGS. 20A and 20B, the magnitude of the electric field and/or potential delivered to the surface (and thus a microorganism on or suspended near the surface) depends in part on the distance between the nozzle tip and the surface. The maximum distance for applying a given electric field to a surface will vary based on the electrical parameters of the control circuit, the applied voltage and waveforms, etc. and the magnitude of the desired field to be delivered. In one example of the hand-held spray device shown in FIGS. 5-6 and 10-14, a suitable electric field was delivered at distances from zero to about eight inches. In other embodiments, a suitable field was delivered at distances of up to six inches. Again, these distances can vary from one embodiment to the next and depending on the type of microorganisms being treated. Suitable ranges for the distance between the nozzle and the surface for effecting irreversible electroporation of one or more microorganisms on the surface include, for example, zero to ten inches, zero to eight inches, zero to six inches, zero to 4 inches and zero to 3 inches. In one example, a desired distance is 3-4 inches.

Experimental test results also showed a correlation between the nozzle/surface distance and the spray duration for removing and killing microorganisms (e.g., bacteria). In general, the closer the nozzle is to the receiving surface, a shorter the spray duration may be. For example, a spray duration of two seconds at a distance ranging from 3-4 inches between nozzles and the receiving surfaces achieved substantial kill results against Escherichia coli (E. coli) and Bacillus bacteria. This is believed to be due to the greater magnitudes of the electric fields and/or potentials that were delivered to the surfaces due to the reduced nozzle/surface distances.

### 13.2 Example 2

### Antimicrobial Efficacy

The efficacy of a spray bottle of Example 2 in reducing bacteria concentrations was also measured. The experiment was performed pursuant to American Society for Testing and Materials (ASTM) E1153-03, established by ASTM International, West Conshohocken, Pa., which is a test method used to evaluate antimicrobial efficacy of sanitizers on inanimate, non-porous, non-food contact surfaces. Separate samples of treated carriers contained Staphylococcus aureus (ATCC #6538) and E. coli (ATCC #11229).

The spray bottle of Example 2 was the same as the spray bottle of Example 1, described above, where the spray bottle of Example 2 was also filled with tap water for the experiment. The test method was modified by spraying the treated carriers for four seconds with the spray bottle of Example 2 at a distance of ranging from three to four inches from the treated carriers, and with an ambient temperature of 20° C. One-third of the treated carriers were then wiped after being sprayed with a wipe to simulate a wiping action, where the wipe used was commercially available under the trade designation “WYPALL” All Purpose Wipes from Kimberly-Clark Corporation, Neenah, Wis. Another third of the treated carriers remained unwiped to measure the efficacy of the spray itself. The final third of the treated carriers were oversprayed, which involved spraying a fine mist in the air, which then deposited onto the treated carriers. Each test was performed in duplicate, referred to as Run 1 and Run 2.

Tables 1 and 2 illustrate the antimicrobial efficacy of the spray bottle of Example 2 respectively against Staphylococcus aureus and E. coli. “CFU” refers to “colony forming unit”, and the “average percent reduction” and the “average log₁₀ reduction” were calculated based on the averages of Runs 1 and 2.

TABLE 1
Staphylococcus Aureus

| | | Log₁₀ | | Average |
|---|---|---|---|---|
| | | CFU/ | Average % | Log₁₀ |
| Example | Test | Carrier | Reduction | Reduction |
| Example 2 | Carrier - Run 1 | <1.6 | >99.999% | >5.2 |
| Example 2 | Carrier - Run 2 | <1.6 | | |
| Example 2 | Wipe - Run 1 | <1.6 | >99.999% | >5.2 |
| Example 2 | Wipe - Run 2 | <1.6 | | |
| Example 2 | Overspray - Run 1 | <1.6 | >99.999% | >5.2 |
| Example 2 | Overspray - Run 2 | <1.6 | | |

TABLE 2
E. coli

| | | Log₁₀ | | Average |
|---|---|---|---|---|
| | | CFU/ | Average % | Log₁₀ |
| Example | Test | Carrier | Reduction | Reduction |
| Example 2 | Carrier - Run 1 | <1.6 | >99.999% | >5.2 |
| Example 2 | Carrier - Run 2 | <1.6 | | |
| Example 2 | Wipe - Run 1 | <1.6 | >99.999% | >5.2 |
| Example 2 | Wipe - Run 2 | <1.6 | | |
| Example 2 | Overspray - Run 1 | <1.6 | >99.999% | >5.2 |
| Example 2 | Overspray - Run 2 | <1.6 | | |

The results shown in Tables 1 and 2 illustrate the efficacy of the spray bottle of the present disclosure for removing and killing a variety of microorganisms. The sprayed carrier (without wiping), the wiped carrier, and the oversprayed carrier each provided an antimicrobial efficacy greater than 99.999% for each of the tested microorganisms.

### 13.3 Examples 3 and 4

### Antimicrobial Efficacy

The efficacy of spray bottles of Examples 3 and 4 in reducing bacteria concentrations was also measured. The experiment was performed in the same manner as discussed above for Example 2, where separate samples of treated carriers contained E. coli O157:H7 (ATCC # 35150), Salmonella enterica (ATCC #10708), Pseudomonas aeruginosa (ATCC #15442), Vancomycin-resistant Enterococcus (VRE) (ATCC #51575), and Methicillin-resistant Staphylococcus aureus (MRSA) (ATCC #33592).

The spray bottles of Examples 3 and 4 were the same as the spray bottle of Example 1, described above, where the spray bottles of Examples 3 and 4 were also filled with tap water for the experiment. The test method was modified by spraying the treated carriers for six seconds with the spray bottles of Examples 3 and 4 at a distance of ranging from three to four inches from the treated carriers, and with an ambient temperature of 21° C. One-third of the treated carriers were then wiped after being sprayed with a wipe to simulate a wiping action, where the wipe used was commercially available under the trade designation “WYPALL” All Purpose Wipes from Kimberly-Clark Corporation, Neenah, Wis. Another third of the treated carriers remained unwiped to measure the efficacy of the spray itself. The final third of the treated carriers were oversprayed, which involved spraying a fine mist in the air, which then deposited onto the treated carriers. Each test was performed in duplicate, referred to as Run 1 and Run 2.

Tables 3-7 illustrate the antimicrobial efficacy of the spray bottles of Examples 3 and 4 against the tested microorganisms, where the “average percent reduction” and the “average log₁₀ reduction” were calculated based on the averages of Runs 1 and 2.

TABLE 3
E. coli O157:H7

| | | Log₁₀ | | Average |
|---|---|---|---|---|
| | | CFU/ | Average % | Log₁₀ |
| Example | Test | Carrier | Reduction | Reduction |
| Example 3 | Carrier - Run 1 | <0.0 | >99.9999% | >6.7 |
| Example 3 | Carrier - Run 2 | <0.0 | | |
| Example 3 | Wipe - Run 1 | <1.6 | >99.999% | >5.1 |
| Example 3 | Wipe - Run 2 | <1.6 | | |
| Example 3 | Overspray - Run 1 | <1.7 | >99.999% | >5.0 |
| Example 3 | Overspray - Run 2 | <1.7 | | |
| Example 4 | Carrier - Run 1 | <0.0 | >99.9999% | >6.7 |
| Example 4 | Carrier - Run 2 | <0.0 | | |
| Example 4 | Wipe - Run 1 | <1.6 | >99.999% | >5.1 |
| Example 4 | Wipe - Run 2 | <1.6 | | |
| Example 4 | Overspray - Run 1 | <1.7 | >99.999% | >5.0 |
| Example 4 | Overspray - Run 2 | <1.7 | | |

TABLE 4
Salmonella Enterica

| | | Log₁₀ | | Average |
|---|---|---|---|---|
| | | CFU/ | Average % | Log₁₀ |
| Example | Test | Carrier | Reduction | Reduction |
| Example 3 | Carrier - Run 1 | 0.8 | >99.9999% | >6.2 |
| Example 3 | Carrier - Run 2 | <0.0 | | |
| Example 3 | Wipe - Run 1 | <1.6 | >99.99% | >4.9 |
| Example 3 | Wipe - Run 2 | <1.6 | | |
| Example 3 | Overspray - Run 1 | <1.7 | >99.99% | >4.9 |
| Example 3 | Overspray - Run 2 | <1.7 | | |
| Example 4 | Carrier - Run 1 | <0.0 | >99.9999% | >6.6 |
| Example 4 | Carrier - Run 2 | <0.0 | | |
| Example 4 | Wipe - Run 1 | <1.6 | >99.99% | >4.9 |
| Example 4 | Wipe - Run 2 | <1.6 | | |
| Example 4 | Overspray - Run 1 | <1.7 | >99.99% | >4.9 |
| Example 4 | Overspray - Run 2 | <1.7 | | |

TABLE 5
Pseudamonas Aeruginosa

| | | Log₁₀ | | Average |
|---|---|---|---|---|
| | | CFU/ | Average % | Log₁₀ |
| Example | Test | Carrier | Reduction | Reduction |
| Example 3 | Carrier - Run 1 | 0.3 | >99.9999% | >6.9 |
| Example 3 | Carrier - Run 2 | <0.0 | | |
| Example 3 | Wipe - Run 1 | <1.6 | >99.999% | >5.6 |
| Example 3 | Wipe - Run 2 | 1.6 | | |
| Example 3 | Overspray - Run 1 | 2 | >99.999% | 5.3 |
| Example 3 | Overspray - Run 2 | 1.7 | | |
| Example 4 | Carrier - Run 1 | <0.0 | >99.9999% | >6.9 |
| Example 4 | Carrier - Run 2 | 0.6 | | |
| Example 4 | Wipe - Run 1 | <1.6 | >99.999% | >5.6 |
| Example 4 | Wipe - Run 2 | <1.6 | | |
| Example 4 | Overspray - Run 1 | 2.3 | >99.99% | 4.7 |
| Example 4 | Overspray - Run 2 | 2.6 | | |

TABLE 6
VRE

| | | Log₁₀ | | Average |
|---|---|---|---|---|
| | | CFU/ | Average % | Log₁₀ |
| Example | Test | Carrier | Reduction | Reduction |
| Example 3 | Carrier - Run 1 | 1.51 | >99.9999% | >5.9 |
| Example 3 | Carrier - Run 2 | <0.0 | | |
| Example 3 | Wipe - Run 1 | <1.6 | >99.999% | >5.1 |
| Example 3 | Wipe - Run 2 | <1.6 | | |
| Example 3 | Overspray - Run 1 | <1.7 | >99.99% | >4.9 |
| Example 3 | Overspray - Run 2 | <1.7 | | |
| Example 4 | Carrier - Run 1 | 0.3 | >99.9999% | >6.5 |
| Example 4 | Carrier - Run 2 | <0.0 | | |
| Example 4 | Wipe - Run 1 | <1.6 | >99.999% | >5.1 |
| Example 4 | Wipe - Run 2 | <1.6 | | |
| Example 4 | Overspray - Run 1 | <1.7 | >99.99% | >4.9 |
| Example 4 | Overspray - Run 2 | <1.7 | | |

TABLE 7
MRSA

| | | Log₁₀ | | Average |
|---|---|---|---|---|
| | | CFU/ | Average % | Log₁₀ |
| Example | Test | Carrier | Reduction | Reduction |
| Example 3 | Carrier - Run 1 | 0.9 | >99.9999% | >6.2 |
| Example 3 | Carrier - Run 2 | <0.0 | | |
| Example 3 | Wipe - Run 1 | <1.6 | >99.999% | >5.1 |
| Example 3 | Wipe - Run 2 | <1.6 | | |
| Example 3 | Overspray - Run 1 | 4.7 | >99.9% | >3.5 |
| Example 3 | Overspray - Run 2 | <1.7 | | |
| Example 4 | Carrier - Run 1 | 1.58 | >99.999% | 5.2 |
| Example 4 | Carrier - Run 2 | 1.38 | | |
| Example 4 | Wipe - Run 1 | <1.6 | >99.999% | >5.1 |
| Example 4 | Wipe - Run 2 | <1.6 | | |
| Example 4 | Overspray - Run 1 | 6.6 | >99.7% | >2.5 |
| Example 4 | Overspray - Run 2 | <1.7 | | |

The results shown in Tables 3-7 illustrate the efficacy of the spray bottle of the present disclosure for removing and killing a variety of microorganisms. For the majority of the results, the sprayed carrier (without wiping), the wiped carrier, and the oversprayed carrier each provided an antimicrobial efficacy greater than 99.999% for each of the tested microorganisms. Several of the overspray runs, such as the overspray runs in Table 7, exhibited high levels of variability between the Run 1 and Run 2. The higher CFU/carriers are believed to be due to improper priming of the spray bottles prior to spraying the treated carriers.

### 13.4 Examples 5 and 6

### Antimicrobial Efficacy

The efficacy of spray bottles of Examples 5 and 6 in reducing concentrations of Influenza A (H1N1) virus was also measured. The experiment was performed pursuant to ASTM E1053-02 and ASTM E1482-04, where samples of treated carriers contained Influenza A (H1N1) virus (ATCC # VR-1469). The treated carriers were also loaded with 5% fetal bovine serum to function as an organic soil load.

The spray bottles of Examples 5 and 6 were the same as the spray bottle of Example 1, described above, where the spray bottles of Examples 5 and 6 were also filled with tap water for the experiment. The test method was modified by spraying the treated carriers for six seconds with the spray bottles of Examples 5 and 6 at a distance of ranging from three to four inches from the treated carriers, and with an ambient temperature of 24° C.

Following the exposure time, the plates were individually scraped with a cell scraper to re-suspend the contents. A 10.6 milliliter aliquot of virus-test substance mixture was recovered from the plate sprayed with the spray bottle of Example 5, and a 11.5 milliliter aliquot of virus-test substance mixture was recovered from the plate sprayed with the spray bottle of Example 6. The recovered mixtures were divided in half and immediately passed through two Sephadex gel filtration columns per unit utilizing the syringe plungers in order to detoxify the mixtures. The filtrates of each test unit were then pooled and titered by 10-fold serial dilution and assayed for infectivity and/or cytotoxicity.

All cell controls were negative for test virus infectivity. The titer of the input virus control was 7.5 log₁₀. The titer of the dried virus control was 6.5 log₁₀. Following exposure to the sprays from the spray bottles of Examples 5 and 6, test virus infectivity was not detected in the virus-test substance mixture for either lot at any dilution tested (≦1.2 log₁₀ for Example 5, and ≦1.3 log₁₀ for Example 6). Test substance cytotoxicity was also not observed in either lot at any dilution tested (≦1.2 log₁₀ for Example 5, and ≦1.3 log₁₀ for Example 6).

The neutralization control (non-virucidal level of the test substance) indicated that the test substance was neutralized at ≦1.2 log₁₀ for Example 5, and ≦1.3 log₁₀ for Example 6. Taking the cytotoxicity and neutralization control results into consideration, as well as the volume of test substance recovered following the exposure time, the reduction in viral titer was ≧5.3 log₁₀ for Example 5 and ≧5.2 log₁₀ for Example 6. Accordingly, under the conditions of tests and in the presence of a 5% fetal bovine serum soil load, the spray bottles of Examples 5 and 6 demonstrated complete inactivation of Influenza A (HINI) virus.

### 13.5 Examples 7 and 8

### Antimicrobial Efficacy

The efficacy of spray bottles of Example 7 and 8 in reducing bacteria concentrations was also measured. The experiment was performed pursuant to the U.S. Environmental Protection Agency (EPA) AOAC Germicidal Spray Method. Separate samples of treated carriers contained MRSA, E. coli, Listeria, Pseudomonas, Salmonella, E. coli O157:H7, and VRE.

The spray bottles of Examples 7 and 8 were the same as the spray bottle of Example 1, described above, where the spray bottles of Examples 7 and 8 were also filled with tap water for the experiment. For each test run for Examples 7 and 8, the test method was modified by spraying the treated carriers for six seconds with the given spray bottle for six seconds with the spray bottle at a distance of ranging from three to four inches from the treated carriers. One-third of the treated carriers were then wiped after being sprayed with a wipe to simulate a wiping action, where the wipe used was commercially available under the trade designation “WYPALL” All Purpose Wipes from Kimberly-Clark Corporation, Neenah, Wis. Another third of the treated carriers remained unwiped to measure the efficacy of the spray itself. The final third of the treated carriers were oversprayed, which involved spraying a fine mist in the air, which then deposited onto the treated carriers.

Each spray bottle test for Examples 7 and 8 was duplicated. In other words, the spray bottle of Example 7 was tested in two runs, and the spray bottle of Example 8 was tested in two runs. Tables 8 and 9 illustrate the antimicrobial efficacy of the spray bottle of Example 7 against the bacteria for Runs 1 and 2, respectively. Correspondingly, Tables 10 and 11 illustrate the antimicrobial efficacy of the spray bottle of Example 8 against the bacteria for Runs 1 and 2, respectively.

TABLE 8
Example 7 - Run 1

| Microorganism | Carrier | Wipe | Overspray |
|---|---|---|---|
| MRSA | 100.00% | 100.00% | poor |
| E. coli | 100.00% | 100.00% | 100.00% |
| Listeria Monocytogenes | 99.99% | 99.99% | poor |
| Pseudamonas Aeruginosa | 100.00% | 100.00% | 100.00% |
| Salmonella Enteritidis | 100.00% | 99.99% | 99.99% |
| E. coli O157:H7 | 100.00% | 100.00% | 100.00% |
| VRE | 100.00% | 100.00% | poor |

TABLE 9
Example 7 - Run 2

| Microorganism | Carrier | Wipe | Overspray |
|---|---|---|---|
| MRSA | 100.00% | 100.00% | 100.00% |
| E. coli | 100.00% | 100.00% | 100.00% |
| Listeria Monocytogenes | 99.99% | 99.99% | 99.99% |
| Pseudamonas Aeruginosa | 100.00% | 100.00% | 100.00% |
| Salmonella Enteritidis | 100.00% | 99.99% | 99.99% |
| E. coli O157:H7 | 100.00% | 100.00% | 100.00% |
| VRE | 100.00% | 100.00% | 100.00% |

TABLE 10
Example 8 - Run 1

| Microorganism | Carrier | Wipe | Overspray |
|---|---|---|---|
| MRSA | 100.00% | 100.00% | 100.00% |
| E. coli | 100.00% | 100.00% | 100.00% |
| Listeria Monocytogenes | 100.00% | 99.99% | 99.99% |
| Pseudamonas Aeruginosa | 100.00% | 100.00% | 100.00% |
| Salmonella Enteritidis | 100.00% | 99.99% | 99.99% |
| E. coli O157:H7 | 100.00% | 100.00% | 100.00% |
| VRE | 100.00% | 100.00% | 100.00% |

TABLE 11
Example 8 - Run 2

| Microorganism | Carrier | Wipe | Overspray |
|---|---|---|---|
| MRSA | 100.00% | 100.00% | poor |
| E. coli | 100.00% | 100.00% | 100.00% |
| Listeria Monocytogenes | 100.00% | 99.99% | 99.99% |
| Pseudamonas Aeruginosa | 100.00% | 100.00% | poor |
| Salmonella Enteritidis | 100.00% | 99.99% | 99.99% |
| E. coli O157:H7 | 100.00% | 100.00% | 100.00% |
| VRE | 100.00% | 100.00% | poor |

The results shown in Tables 8-11 further illustrate the efficacy of the spray bottle of the present disclosure for removing and killing a variety of different bacteria. As shown, the spray carrier and the spray/wiping combination each provided an antimicrobial efficacy of 99.999% for each of the tested bacteria. Furthermore, the results of the overspray provided an antimicrobial efficacy of 99.99% for most of the tested bacteria. The samples that provided poor antimicrobial efficacies are believed to be due to a lack of conductivity due to the overspray, which effectively eliminates the conductive conduit. This further shows that the conductivity generated from the spray bottle is providing the antimicrobial activity, rather than the water or solution produced from the electrolysis cell.

### 13.6 Examples 9-11

### Antimicrobial Efficacy

The efficacy of spray bottles of Example 9-11 in reducing bacteria concentrations was also measured pursuant to the same procedure described above for Example 2, except that the sprayed samples were not wiped. Separate samples of treated carriers contained E. coli O157:H7, Salmonella enteritidis, and Listeria monocytogenes. In comparison to the spray bottle of Example 2, which was filled with tap water, the spray bottles of Examples 9-11 were filled with water having different mineral concentrations. Tables 12-14 list the types of water supplied during various runs with the spray bottles of Examples 9-11 and with the spray bottle of Comparative Example A. The spray bottle of Comparative Example A incorporated an electrolysis cell for electrochemically activating the water, but did not include an electroporation electrode for generating and electric field through the sprayed water.

The “Bottled Water with Salt” was a mixture of 0.25% by volume sodium chloride in bottled water commercially available under the trade designation “FIJI” Natural Artesian Water from FIJI Water Company, LLC, Los Angeles, Calif. The “Tap Water” was standard tap water attained in Minneapolis, Minn. The “Tap Water with Salt” was a mixture of 0.25% by volume sodium chloride in the Tap water. The “Distilled Water” was a standard distilled water. Tables 12-14 illustrate the antimicrobial efficacy of the spray bottles of Examples 9-11 against E. coli O157:H7, Salmonella enteritidis, and Listeria monocytogenes, respectively.

TABLE 12
E. coli O157:H7

| | Bottle Water | Tap | Tap Water | Distilled |
|---|---|---|---|---|
| Example | with Salt | Water | with Salt | Water |
| Comparative | 99% | 0% | 99.9% | 0% |
| Example A | | | | |
| Example 9 | 99.999% | 99.999% | 99.999% | 99.9% |
| Example 10 | 99.999% | 99.999% | 99.999% | 99.9% |
| Example 11 | 99.9999% | 99.999% | 99.999% | 99.9% |

TABLE 13
Salmonella Enteritidis

| | Bottle Water | Tap | Tap Water | Distilled |
|---|---|---|---|---|
| Example | with Salt | Water | with Salt | Water |
| Comparative | 99.9% | 99.9% | 99.9% | 0% |
| Example A | | | | |
| Example 9 | 99.999% | 99.99% | 99.99% | 99.99% |
| Example 10 | 99.999% | 99.99% | 99.999% | 99.99% |
| Example 11 | 99.999% | 99.99% | 99.999% | 99.99% |

TABLE 14
Listeria Monocytogenes

| | Bottle Water | Tap | Tap Water | Distilled |
|---|---|---|---|---|
| Example | with Salt | Water | with Salt | Water |
| Comparative | 99.99% | 99% | 99.99% | 0% |
| Example A | | | | |
| Example 9 | 99.9999% | 99.999% | 99.9999% | 99.99% |
| Example 10 | 99.9999% | 99.999% | 99.9999% | 99.99% |
| Example 11 | 99.9999% | 99.999% | 99.9999% | 99.99% |

Each of the tested samples for Examples 9-11 achieved greater than a 99.99% reduction for each of the bacteria tested with the Bottled Water with Salt, the Tap Water, and the Tap Water with Salt, and exhibited greater killing efficacy compared to the results of Comparative Example A. This is particularly true with the Distilled Water, where the tested samples of Comparative Example A was ineffective in reducing the bacteria. Accordingly, the electroporation attainable with the spray bottle of the disclosure is capable of effectively removing and killing a variety of bacteria from surfaces, regardless of the mineral content of the water used with the spray bottle.

### 13.7 Example 12

### Water Analysis

The water used in the spray bottle of Example 1 was also measured to identify its pH, conductivity, and the concentrations of sodium, calcium, and magnesium ions in the water samples. The pH of the water was measured using a calibrated pH probe and meter. The conductivity of the water was measured using a calibrated one-centimeter conductivity probe and meter. The concentrations of the sodium, calcium, and magnesium ions in the water were determined using an Inductively Coupled Plasma—Atomic Emission Spectrometer pursuant to EPA Method 200.7. Additionally, the Total Hardness of the water was calculated from the determined calcium and magnesium concentrations pursuant to Equation 1:

Total Hardness=2.497*[calcium]+4.116*[magnesium] (Equation 7)

where the Total Hardness of the water is in milligrams/liter (mg/L) of CaCO₃, [calcium] is the concentration of calcium in the water in mg/L, and [magnesium] is the concentration of magnesium in the water in mg/L. Table 15 illustrates the measured pH, conductivity in microSiemens (μS), concentrations of sodium, calcium, and magnesium ions in parts-per-million (ppm), and the Total Hardness of the water in ppm.

TABLE 15

| Property | Results | |
|---|---|---|
| pH | 7 | |
| Conductivity | 1280 | μS |
| Sodium concentration | 167 | ppm |
| Calcium concentration | 19 | ppm |
| Magnesium concentration | 6 | ppm |
| Total Hardness | 73 | ppm CaCO₃ |

### 14. Example Uses in Various Industries

One or more of the examples and embodiments disclosed herein, or modifications thereof, can be implemented in the following industries and/or applications, which are provided as non-limiting examples:

A. Industrial Cleaning & Disinfection:

### Surface Cleaning & Disinfection

### Removal of Bio-Film & Algae

### Effective Biocide

### Clean-in-Place [CIP] Sanitizing & Disinfection

B. Health & Medical Care:

### Cold Sterilization of Medical Instruments

### Surface Cleaning & Disinfection.

### Production of Sterile Water

Linen disinfection when washed

### Fogging Disinfection of Air & Clean Rooms

C. Veterinarian Applications:

Increased vitality and disease resistance
Residue-free treatment of Infection and wound care
Increased nutritional benefit of food

D. Poultry Industry:

### General Disinfection.

### Surface Cleaning & Fog Misting Medium for Aerobic Bacteria

Elimination of pathogens in drinking water
Lice & Other Pest Control on feathers
Fog Misting to destroy Aerobic & Anaerobic Bacteria.
Equipment cleaning without further additives

E. Horticulture/Agriculture:

### Suppression of Pathogenic Fungi on Plants

### Disinfection of Irrigation Water for Crop Spraying & Pest Control.

Decreased Toxicity of Effluent Filtration into Water Aquifers

### Prolonged Shelf-Life of Vegetables, Fruit & Cut Flowers

Disinfection of seeds, stimulation and acceleration of plant growth with increased yield

### Disinfection of Stored Grain

F. Water, Waste Water & Sewage Treatment.

### Disinfect Municipal Effluent

### Neutralize Water

### Removal of Bio-Film & Algae

### Neutralize Odor Compounds

### Reduce Formation of Toxic By-Products.

### 15. Further Suspension Mechanisms

Another aspect of the disclosure relates to a process for deactivating or destroying microorganisms, by applying a potential or electrochemical pressure to microorganisms, in a medium that is capable of suspending the microorganisms using alternative and/or additional suspension mechanisms. As discussed above, such as for spray bottles 10, 300, 500 and/or any of the other apparatus 1200, 1300, 1400, 1500 described herein, microorganism suspension can be accomplished with electrochemically-activated liquids produced by one or more electrolysis cells. In addition, microorganisms can be suspended in the medium (e.g., a liquid) with use of chemical compounds, such as suspension additives (e.g., detergent surfactants), liquid-activating materials (e.g., zeolites), and the like. As discussed below, these materials are configured to treat a liquid to increase its suspension properties. The suspension additive(s) can be used in addition to or in replace of an electrolysis cell for promoting increased suspension of microorganisms in the liquid distributed from the apparatus, for example.

### 15.1 Suspension Additives

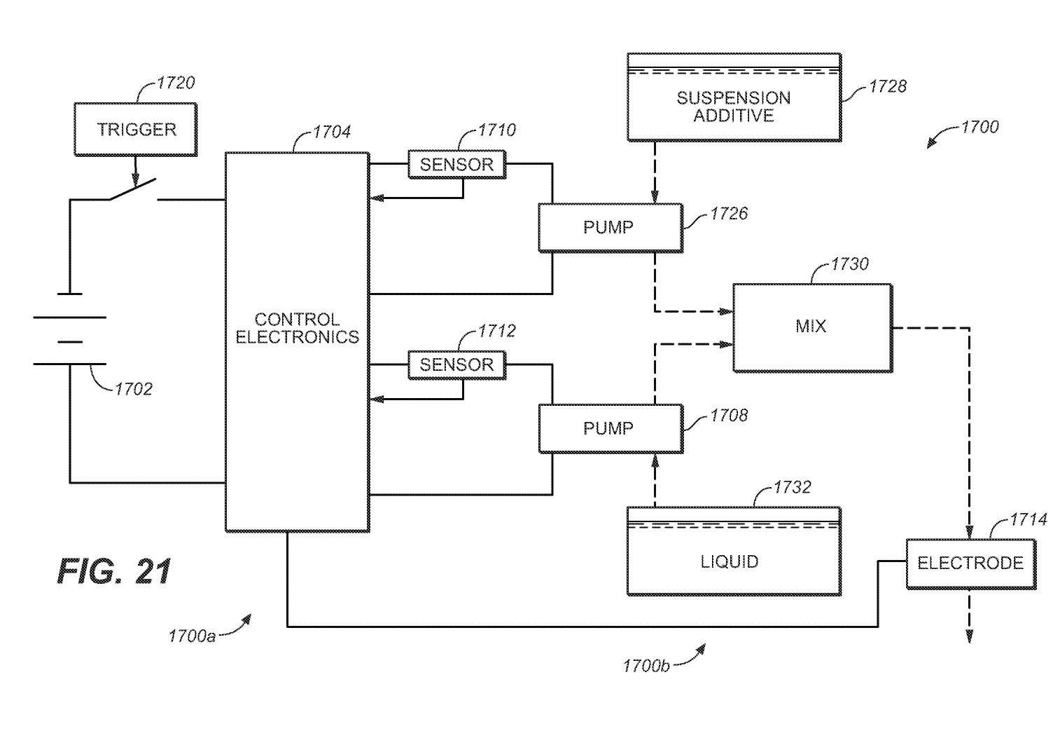

FIG. 21 is a diagram illustrating system 1700 according to an example embodiment of the disclosure, which can be incorporated into any of the embodiments disclosed herein, for example. System 1700 includes electrical subsystem 1700a and fluid subsystem 1700b, where electrical subsystem 1700a may function in the same manner as system 1600 (shown in FIG. 19), for example, and where the corresponding reference labels are increased by “100”. In the embodiment shown in FIG. 20, however, the component corresponding to electrolysis cell 1606 is replaced with pump 1726 for feeding a suspension additive from reservoir 1728 to mixing chamber 1730. This arrangement also allows pump 1708 to feed a liquid (e.g., tap water) from reservoir 1732 to mixing chamber 1730 to mix the suspension additive in the liquid. The components corresponding to LEDs 1622 and 1624 are omitted in FIG. 20 for ease of discussion. The suspension additive may be added to the liquid at any other location along the liquid flow path, such as directly in reservoir 1732, and may be mixed by any suitable method, with or without a pump, and/or supplied as part of the liquid introduced into reservoir 1732, for example.

The suspension additive (such as that in reservoir 1728) desirably includes one or more chemical compounds configured to assist in suspending particles and microorganisms in the liquid dispensed from reservoir 1732. As discussed above, the suspension mechanism may alter the ORP of the dispensed liquid (producing dispensed liquid having a positive ORP, a negative ORP or a combination of both). These enhanced cleaning effects can serve to suspend particles and microorganisms above the surface within the dispensed liquid, for example. Suitable chemical compounds for use in the suspension additive include, for example, compounds configured to reduce the surface tension of the liquid, such as surfactants (e.g., detergent surfactants).

Examples of suitable surfactants for use in the suspension additive include anionic, non-ionic, and cationic surfactants. Examples of anionic surfactants include alkyl sulfates, alkyl sulfonates, sulfosuccinates, and combinations thereof. Examples of suitable alkyl sulfates include primary and secondary alkyl sulfates, alkyl ether sulphates, fatty alcohol sulfates, and combinations thereof. Examples of suitable alkyl chain lengths for the alkyl sulfates range from C8 to C15 (e.g., C8 to C15 primary alkyl sulphates). Examples of suitable alkyl sulfonates include alkyl benzene sulfonates (e.g., linear alkyl benzene sulfonates with C8 to C15 alkyl chain lengths), alkyl xylene sulfonates, fatty acid ester sulfonates, and combinations thereof. Examples of suitable sulfosuccinates include dialkyl sulfosuccinates.

Examples of nonionic and cationic surfactants include alcohol ethoxylates (e.g., alkyl phenoxy polyethoxy ethanols), alkyl polyglycosides, polyhydroxyamides, monoethanolamine, diethanolamine, triethanolamine, glycerol monoethers, alkyl ammonium chlorides, alkyl glucosides, polyoxyethylenes, and combinations thereof.

The suspension additive may also include one or more additional materials to assist in the suspension and cleaning properties. Examples of suitable additional materials include oxidants, enzymes, defoaming agents, colorants, optical brighteners, corrosion inhibitors, perfumes, antimicrobial agents, anitbacterial agents, antifungal agents, pH modifiers, solvents, and combinations thereof. The additive materials may provide longer residence times and greater sanitizing effect on some surfaces, such as porous surfaces. For example, the additive materials may reside on a surface after the electric field (from electroporation electrode 1714) is removed.

The suspension additive may be provided to reservoir 1728 (and/or reservoir 1732) in a variety of media, for example fluids, solutions, pellets, blocks, powders, and the like. In the shown embodiment, the suspension additive is desirably a solution of the surfactant(s) and additional materials dissolved or otherwise suspended in a carrier medium (e.g., water).

During operation, when trigger 1720 is depressed, control electronics 1704 is enabled and generates appropriate voltage outputs for driving pumps 1708 and 1726 and electroporation electrode 1714. The relative feed rates of pumps 1708 and 1726 may vary depending on the desired concentration of the suspension additive in the liquid. Each of the pumps may include, for example, a controller that controls the operation of the pump through a control signal, for example. In accordance with one exemplary embodiment, the control signal can include a pulsed signal that provides power relative to ground and controls the duration over which the pump drives the suspension additive through mixing chamber 1730. Other types of control signals and control loops (open or closed) can be used. In addition, one or both of pumps 1726 and 1708 can be eliminated and the liquid and/or suspension additive can be fed by another mechanism, such as gravity. In addition, the operation of pumps may be monitored by current sensors 1710 and 1712, for example.

As discussed above, the suspension additive and the liquid are combined (such as in mixing chamber 1730) to form a solution. Mixing chamber 1730 may include a variety of geometries and designs configured to assist in the mixing process (e.g., baffled walls). Other examples of suitable mixing devices includes a Venturi tube and merging flow paths. The relative concentrations of surfactant(s) in the suspension additive (such as from reservoir 1728) and the liquid from reservoir 1732 may vary on the concentration of the surfactant(s) in the suspension additive and the relative feed rates, for example. Accordingly, upon exiting mixing chamber 1730 (and/or from a pre-mixed solution from reservoir 1732), the solution desirably includes a surfactant concentration that is great enough to suspend particles and/or microorganisms in the dispensed solution. Examples of suitable surfactant concentrations in the solution upon exiting mixing chamber 1730 (and/or reservoir 1732) range from about 0.1% by volume to about 15% by volume, with particularly suitable surfactant concentrations ranging from about 0.5% to about 10% by volume.

The resulting solution may exit mixing chamber 1730 (and/or reservoir 1732 for example) and come into contact with electroporation electrode 1714 prior to being dispensed (e.g., sprayed) onto a surface or volume and/or upon being dispensed. The suspension additive can serve to suspend particles and microorganisms above the surface within the dispensed solution. In particular, while not wishing to be bound by theory, it is believed that at least a portion of the surfactant(s) of the suspension additive, which contain hydrophobic and hydrophilic molecular chain ends, can reside at the liquid/surface/gas interfaces. As such the hydrophilic chain ends reside within the liquid and the hydrophobic chain ends extend out of the liquid, thereby reducing the surface tension of the liquid. When the hydrophobic chain ends contact particles and microorganisms on the surface, they can entrap and suspend the particles/microorganisms above the surface within the dispensed solution. Furthermore, in some embodiments, the surfactants can increase the potency of the liquid, and assist in penetrating the structures of the microorganisms.

As discussed above, electroporation electrode 1714 may apply an electric field through the solution to the surface, which can be sufficient to cause irreversible electroporation of (or otherwise inactivate or damage) the suspended microorganisms. A suspension additive in the solution allows the microorganisms to be suspended above the surface in the same or similar manner to an altered ORP that is achieved with an electrolysis cell, for example. By separating the microorganisms from the surface, for example, such that they are suspended in the solution above the surface, the electric field produced along the surface by electroporation electrode 1714 is applied more easily across the microorganism cells. Whereas, if the microorganism is in contact with the surface, the electric field is more easily discharged into the surface ground and may be less effective in creating irreversible electroporation of the organisms cells. With the cell suspended, the applied alternating field, for example, oscillates back and forth causing damage to the cells.

While illustrated in use with system 1700, suspension additives may be used with any of the embodiments of the disclosure. For example, the suspension additive may be introduced into reservoir 12 of spray bottle 10 (shown in FIG. 1) and in container 510 of spray bottle 500 (shown in FIGS. 10A-10C) in a batch manner when filling reservoir 12 with the liquid (and/or supplied from a separate reservoir carried by the apparatus). Furthermore, system 1700 may also be used in cleaner 1200 (shown in FIG. 15), surface cleaning assembly 1300 (shown in FIG. 16), flat mop 1400 (shown in FIG. 17), device 1500 (shown in FIG. 18), system 1600 (shown in FIG. 19), and the like. In these embodiments, the electrolysis cells (e.g., electrolysis cells 18, 552, 1208, and 1606) may be omitted. Alternatively, the electrolysis cells may be used in conjunction with the suspension additive to further increase the suspension of particles and microorganisms in the dispensed solution.

### 15.2 Liquid-Activating Materials

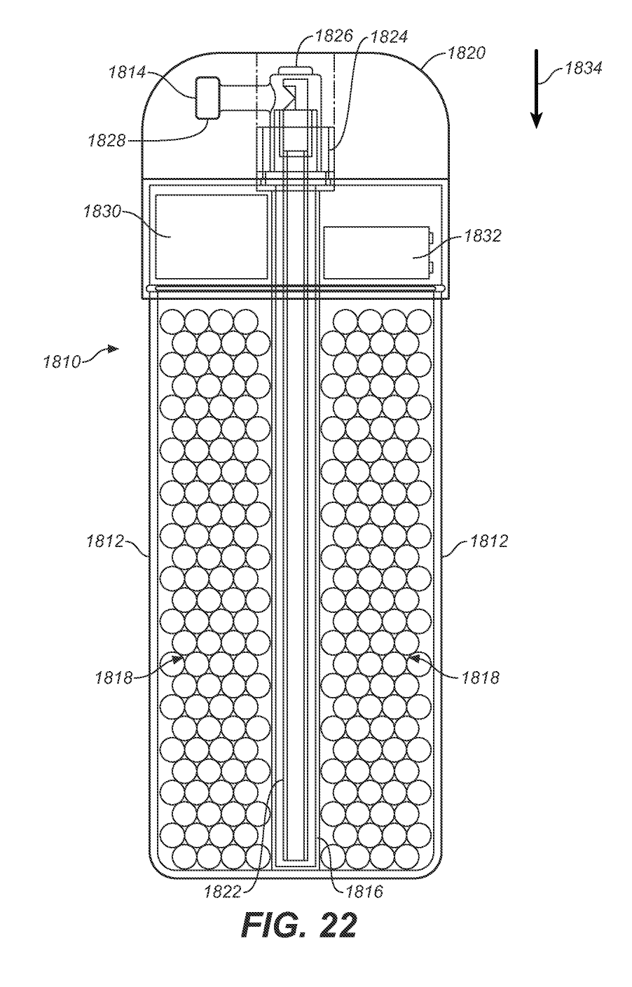

FIG. 22 is a schematic illustration of spray bottle 1810, which is an example of a hand-held spray device that is configured to retain one or more liquid-activating materials (e.g., zeolites) for altering the ORP of liquids retained and dispensed by spray bottle 1810. In another example, the spray device may form part of a larger device or system. In the embodiment shown in FIG. 22, spray bottle 1810 includes reservoir 1812, which is defined by a base housing of spray bottle 1810, and is configured to contain a liquid to be treated and then dispensed through nozzle 1814. Additionally, reservoir 1812 may contain filter 1816 and media 1818, where media 1818 compositionally includes one or more liquid-activating materials. Filter 1816 is a media filter configured to allow the liquid to pass through, but desirably prevents the macrosized particles of media 1818 from passing through. Reservoir may, for example, be configured as a replaceable cartridge that is engageable and disengageable with 1820.

Examples of suitable liquid-activating materials for use in media 1818 include porous minerals, such as porous aluminosilicate minerals (e.g., zeolites). Examples of suitable zeolites for use in media 1818 include hydrated and anhydrous structures of aluminosilicate minerals, which may contain one or more of sodium (Na), potassium (K), cerium (Ce), calcium (Ca), barium (Ba), strontium (Sr), lithium (Li), and magnesium (Mg). Examples of suitable zeoiltes for use in media 1818 include analcime, amicite, barrerite, bellbergite, bikitaite, boggsite, brewsterite, chabazite, clinoptilolite, cowlesite, dachiardite, edingtonite, epistilbite, erionite, faujasite, ferrierite, garronite, gismondine, gobbinsite, gmelinite, gonnardite, goosecreekite, harmotome, heulandite, laumontite, levyne, mazzite, merlinoite, montesommaite, mordenite, mesolite, natrolite, offretite, paranatrolite, paulingite, perlialite, phillipsite, pollucite, scolecite, stellerite, stilbite, thomsonite, tschernichite, wairakite, wellsite, willhendersonite, yugawaralite, anhydrous forms thereof, and combinations thereof. Examples of commercially available zeolites for use in media 1818 include clinoptilolites from KMI Zeolite, Inc., Sandy Valley, Nev., which have an average density of about 2.3 grams/cubic-centimeter and a nominal particle sizing of +40 mesh.

Non-zeolite materials or mechanisms may also be utilized. Examples of suitable non-zeolite minerals for use in media 1818 include resins, apophyllite, gyrolite, hsianghualite, kehoeite, lovdarite, maricopaite, okenite, pahasapaite, partheite, prehnite, roggianite, tacharanite, tiptopite, tobermorite, viseite, and combinations thereof. Examples of suitable resins include ion-exchange resins, such as those having cross-linked aromatic structures (e.g., cross-linked polystyrene) containing active groups (e.g., sulfonic acid groups, amino groups, carboxylic acid groups, and the like). The ion-exchange resins may be provided in a variety of media, such as in resin beads, for example. These non-zeolite minerals may be used in combination with or as alternatives to the zeolites in media 1818.

Media 1818 may be provided in a variety of media forms, such as in ceramic balls, pellets, powders, and the like. While retained in reservoir 1812, media 1818 treats the retained liquid, thereby imparting a negative ORP (and/or a positive ORP) on the retained liquid by ion exchange, for example. Media 1818 desirably imparts a negative ORP to the liquid of at least about of −50 mV and/or a positive ORP of at least about +50 mV. In another example, media 1818 imparts a negative ORP to the liquid of at least about of −100 mV and/or a positive ORP of at least about +100 mV. As discussed above, altering the ORP allows the dispensed treated liquid to suspend particles and microorganisms.

Spray bottle 1810 also includes cap housing 1820, tube 1822, pump 1824, actuator 1826, electroporation electrode 1828, circuit board and control electronics 1830, and batteries 1832. Cap housing 1820 desirably seals reservoir 1812 when closed, and may be depressed in the direction of arrow 1834 by a user to engage actuator 1826. Batteries 32 can include disposable batteries and/or rechargeable batteries, for example, or other appropriate portable or corded electrical source in addition to or in place of batteries, to provide electrical power to electroporation electrode 1828 when energized by circuit board and control electronics 30. In one embodiment, pump 1824 may also be electrically powered.

Pump 1824 draws liquid from reservoir 1812 through filter 1816 and tube 1822, and forces the liquid out nozzle 1814. While passing through nozzle 1814, the liquid contacts electroporation electrode 1828. As discussed above, electroporation electrode 1828 may apply a voltage (such as an alternative voltage) to the dispensed solution, creating an electric field through the dispensed solution to the surface, which can be sufficient to cause damage to the suspended microorganisms, such as by irreversible electroporation. The altered ORP of the dispensed liquid allows the microorganisms to be suspended above the surface in the same or similar manner to an altered ORP that is achieved with an electrolysis cell, for example. By suspending the microorganisms from the surface, for example such that they are suspended in the solution above the surface, the electric field produced along the surface by electroporation electrode 1828 is applied more easily across the microorganism cells. With the cell suspended, the applied alternating field oscillates back and forth causing damage to the cells, as discussed above.

While illustrated in use with system 1810, media 1818 may be used with any of the embodiments of the disclosure. For example, the suspension additive may be introduced into reservoir 12 of spray bottle 10 (shown in FIG. 1) and in container 510 of spray bottle 500 (shown in FIGS. 10A-10C) in a batch manner, for example, when filling reservoir 12 with the liquid. In these embodiments, the electrolysis cells (e.g., electrolysis cells 18 and 552) may be omitted. Alternatively, the electrolysis cells may be used in conjunction with media 1818 to further increase the suspension of particles and microorganisms in the dispensed solution.

In a further example, the reservoir 1812 may include a fill port or opening that may be used to fill (and/or refill) the reservoir with the liquid and/or media 1818. In yet a further example, bottle 1810 may include a fitting for receiving liquid from an external source, such as through a hose, wherein the liquid flows through media 1818.

Furthermore, media 1818 may also be used in cleaner 1200 (shown in FIG. 15), surface cleaning assembly 1300 (shown in FIG. 16), flat mop 1400 (shown in FIG. 17), device 1500 (shown in FIG. 18), system 1600 (shown in FIG. 19), and the like.

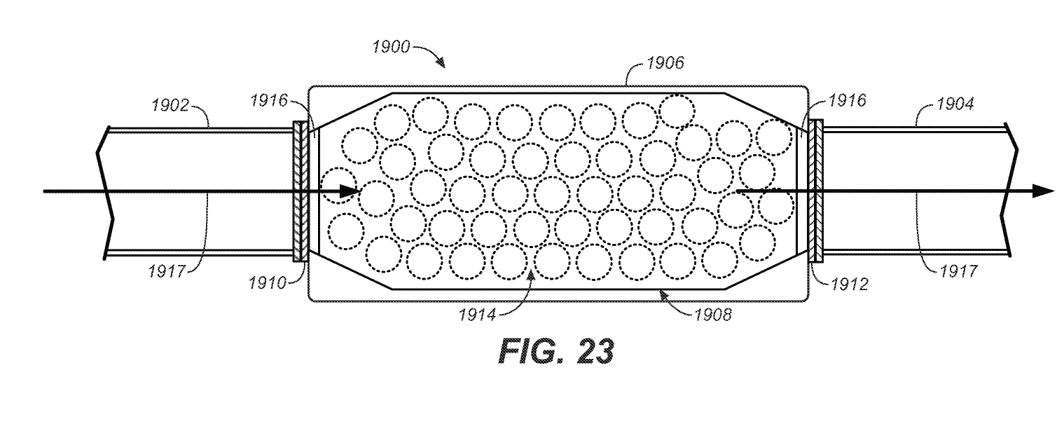

FIG. 23 is a schematic diagram of a cartridge 1900 that may be installed, for example, in a fluid line of a flow-through system, such as between fluid line segments 1902 and 1904. Cartridge 1900 may be positioned at any suitable location along the flow paths on any of the apparatus described herein, such as cleaner 1200 (shown in FIG. 15), surface cleaning assembly 1300 (shown in FIG. 16), flat mop 1400 (shown in FIG. 17), device 1500 (shown in FIG. 18), system 1600 (shown in FIG. 19), spray bottle 10 (shown in FIG. 1), spray bottle 300 (shown in FIG. 8), spray bottle 500 (shown in FIGS. 10A-10C), and spray bottle 1810 (shown in FIG. 22).

In the embodiment shown in FIG. 23, cartridge 1900 includes housing 1906, which defines interior chamber 1908, and interfaces 1910 and 1912. Interfaces 1910 and 1912 desirably allow cartridge 1900 to mate respectively with fluid line segments 1902 and 1904 in a manner that is lockable and unlockable, or otherwise removably engagable. This arrangement allows multiple cartridges to interchangably mate with fluid line segments 1902 and 1904. For example, when a cartridge 1900 eventually expires over multiple uses, the expired cartridge 1900 may be removed from fluid line segments 1902 and 1904, and replaced with a fresh cartridge 1900. Interfaces 1910 and 1912 can also include simple male and/or female fittings.

Interior chamber 1908 retains media 1914 for treating liquids passing through cartridge 1900 with the use of media filters 1916, where the flow of the liquids through cartridge is represented by arrows 1917). Suitable materials for media 1914 include those discussed above for media 1818 (shown in FIG. 22), for example. Accordingly, media 1914 treats the liquid flowing through interior chamber 1908, thereby imparting a negative ORP (and/or a positive ORP) on the flowing liquid by ion exchange. The volume of interior changer 1908 and the amount of media 1914 within interior chamber 1908 are desirably selected to provide a suitable residence time of the flowing liquid to sufficiently alter the ORP. These parameters may vary depending on the volumetric flow rate of the liquid through fluid line segments 1902 and 1904. In a further example, media 1914 is contained in one or more of the liquid reservoirs/tanks carried by the various apparatus described herein, such as cleaner 1200 (shown in FIG. 15), surface cleaning assembly 1300 (shown in FIG. 16), flat mop 1400 (shown in FIG. 17), device 1500 (shown in FIG. 18), system 1600 (shown in FIG. 19), and the like.

Media 1914 desirably imparts a negative ORP to the liquid of at least about of −50 mV and/or a positive ORP of at least about +50 mV, and in another embodiment at least about of −100 mV and/or a positive ORP of at least about +100 mV. As discussed above, altering the ORP allows the dispensed treated liquid to suspend particles and microorganisms. The treated liquid may then exit interior chamber 1908 into fluid line segment 1904 to be dispensed from the system, such as discussed above for cleaner 1200, surface cleaning assembly 1300, flat mop 1400, device 1500, system 1600, and the like.

Interchangeable cartridges or other supply containers of media 1818 and/or 1914 may be configured in many different ways to engage with and disengage from the particular apparatus with which it is used. For example, with the spray bottle embodiments of the disclosure, the base housings of spray bottles 10, 500 and 1810 (respectively containing reservoir 12, container 510, reservoir 1812) may be removably engageable with the head portion (and/or any other portion) of the respective spray bottle, thereby allowing multiple cartridge base portions to interchangeably mate with a single head portion. In another example, any part of the spray bottles, such as the base portions or head portions may be configured to removably engage a cartridge of media 1818 and/or 1914. In a further example, the spray bottle can be configured to engage such a cartridge within the base of the bottle or at the head of the bottle, such as at base 502 and/or at the location of electrolysis cell 552 in the head portion of spray bottle 500 shown in FIGS. 10A-10C. The replaceable cartridges may be configured to allow multiple interchangeable cartridges to readily mate with, and disengage from, the fluid lines of the spray bottle, for example.

In one particular example, the base of a spray bottle is configured to receive a cylindrical cartridge containing media 1818, 1914. For example, looking at FIG. 1, the reservoir 12 of bottle 10 (shown in FIG. 1) can be modified to eliminate electrolysis cell 18 and to include a circular opening within the base of the reservoir to receive a cylindrical cartridge. One end of the cylindrical cartridge is insertable along its longitudinal axis into the opening. The opposite end may include an appropriate latch and sealing mechanism. For example, the bottom end of the cartridge may have an annular shoulder with an o-ring that seals against the bottom of reservoir 12, about a circumference of the opening, when the cylindrical cartridge is fully inserted into the reservoir so as to seal the interior of the reservoir about the base of the cylindrical cartridge. The length of the cartridge may extend into the reservoir by any suitable distance, such as but not limited to half or a third of the height of the reservoir. The cartridge can have any suitable mechanism to lock the cartridge into place, such as by rotating the cartridge about its axis upon insertion. Examples include mating threads and other locking mechanisms.

The walls of the cylinder can have any suitable configuration to permit interaction between the media 1818, 1914 contained within the cartridge and the liquid contained in the reservoir. For example, the cylinder may include one or more apertures sufficient to allow the liquid to pass into the interior cavity of the cylindrical cartridge. In a particular example, the side walls have a plurality of apertures formed by openings in a mesh, screen, and/or perforated side wall, for example.

The apertures may be closed, for example, when not in use, such as before insertion, to reduce potential contamination of the media contained in the cartridge. In one example, the cartridge may be supplied with a removable film or sleeve that covers the apertures during storage. This film or sleeve may be removed prior to (or after) insertion of the cartridge into the base of the bottle. In another example, the cartridge is configured with a sealing mechanism that automatically seals the one or more apertures when the cartridge is not inserted into and/or engaged with the bottle. For example, the cartridge may include an inner cylindrical side wall and an outer cylindrical sleeve that is coaxial with and movable relative to the inner cylindrical side wall. The inner cylindrical side wall contains the media 1818, 1914 and has the one or more apertures discussed above. The outer cylindrical sleeve is movable, such as in a circumferential or axial direction, between a closed position and an open position. In the closed position, the cylindrical sleeve covers one or more of the apertures of the inner cylindrical side wall so as to seal the interior cavity of the cartridge from contamination, for example. In the open position, the outer cylindrical sleeve uncovers one or more of the apertures in the inner cylindrical side wall. For example, the outer cylindrical sleeve covers one or more of the apertures of the inner cylindrical side wall so as to seal the interior cavity of the cartridge from contamination, for example. In one embodiment, the cylindrical outer sleeve includes a plurality of apertures that align with the apertures in the inner cylindrical side walls when in the open position. In the closed position, the apertures in the outer cylindrical sleeve do not align with the apertures in the inner cylindrical side wall such that the material of one cylinder seals or otherwise covers the apertures in the other cylinder. Many other arrangements and constructions for engaging a cartridge with a reservoir are possible and contemplated in the present disclosure.

Movement between the open and closed position may be manual or automatic, for example. In one embodiment, the outer sleeve is biased into the closed position, by a mechanism, such as a spring action. Upon insertion into the reservoir, the outer sleeve is biased into the open position, such by a lever or surface engagement with the reservoir or other element, for example.

Similarly, in embodiments in which media 1818, 1914 is used in apparatus such as cleaner 1200 (shown in FIG. 15), surface cleaning assembly 1300 (shown in FIG. 16), flat mop 1400 (shown in FIG. 17), device 1500 (shown in FIG. 18), system 1600 (shown in FIG. 19), and the like, the media may be contained in replaceable cartridges, for example. These cartridges may be configured to allow multiple interchangeable cartridges to readily mate with, and disengage from, the fluid lines of the apparatus. For example, the cartridge may be accessible/insertable from an interior of the apparatus or from an exterior of the apparatus. In one example, the cartridge is accessible/insertable through a side wall of the apparatus.

In embodiments incorporating media 1818 and/or media 1914, for example, electrolysis cells (e.g., electrolysis cells 18, 552, 1208, and 1606) may be omitted. Alternatively, the electrolysis cells may be used in conjunction with a further suspension mechanism to further increase the suspension of particles and microorganisms in the dispensed solution. The use of further (or alternative) suspension mechanisms, such as suspension additives (e.g., detergent surfactants) and liquid-activating materials (e.g., zeolites), increases the versatility of the systems discussed herein for suspending particles and microorganisms in dispensed liquids for use with a sanitization process such as, for example, by electroporation.

An aspect of the disclosure relates to an apparatus comprising: a container configured to engage a liquid and at least one compound configured to increase suspension properties of the liquid to provide a treated liquid; a liquid flow path coupled to the container; a liquid dispenser coupled in the liquid flow path, adapted to dispense the treated liquid to a surface or volume of space; an electrode electrically coupled to the liquid flow path; and a control circuit adapted to generate an alternating electric field between the electrode and the surface or volume of space, through the dispensed treated liquid, without a corresponding return electrode.

The container can include but is not limited to any suitable container such as various elements described herein as containers, reservoir, tanks, chambers, cartridges, compartments, etc, for example. For example, the container can include a liquid source container (for example containers 12, 510, 1206, 1406, 1732, 1812), an additive container (for example container 1728), a mixing chamber 1730, cartridge 1900 (flow-through and/or source, for example), compartment 1408, etc., merging fluid lines, etc.

The container may engage a liquid with at least one compound in any suitable manner, including but not limited to active and/or passive mixing, blending, combining, etc.; containing; and/or enabling interaction, contact and/or reaction between. For example, engagement may include a pre-mixed solution of the liquid and the compound being contained in a container. In another example, the container may enable a liquid to engage a least one compound supplied from a separate source, such as in a mixing chamber, for example. In another example the container may enable interaction between a liquid and at least one compound within a flow-through and/or source cartridge. Other arrangements are also envisioned.

At least one compound can include but is not limited to at least one surfactant, at least one liquid-activating material. At least one liquid-activating material can include, but is not limited to a material selected from the group including zeolites, ion-exchange resins, and combinations thereof.

Although the present disclosure has been described with reference to one or more embodiments, workers skilled in the art will recognize that changes may be made in form and detail without departing from the scope of the disclosure and/or the issued claims appended hereto. Also while certain embodiments and/or examples have been discussed herein, the scope of the invention is not limited to such embodiments and/or examples. One skilled in the art may implement variations of these embodiments and/or examples that will be covered by one or more issued claims appended hereto.

What is claimed is:

1. An apparatus comprising:
a mobile body configured to travel over a surface;
a first liquid line configured to receive a liquid;
a container coupled to the first liquid line, the container retaining media of at least one liquid-activating material, the media of the at least one liquid-activating material being configured to modify an oxidation reduction potential of the received liquid to provide a treated liquid;
a second liquid line coupled to the container and configured to receive the treated liquid from the container; and
a dispenser operably coupled to the second liquid line at a location downstream from the container, the dispenser being configured to dispense the treated liquid.

2. The apparatus of claim 1, wherein the at least one liquid-activating material comprises a material selected from the group consisting of zeolites, ion-exchange resins, and combinations thereof.

3. The apparatus of claim 1, and further comprising a reservoir configured to retain the liquid, the reservoir being coupled to the first liquid line at a location upstream from the container.

4. The apparatus of claim 1, wherein the container comprises a cartridge that is configured to be removably engaged with the first liquid line and the second liquid line.

5. The apparatus of claim 4, wherein the cartridge comprises:
a housing that defines an interior chamber configured to retain the media;
a first housing interface configured to provide access to the interior chamber, and further configured to be removably engaged with first liquid line; and
a second housing interface configured to provide access to the interior chamber, and further configured to be removably engaged with the second liquid line.

6. The apparatus of claim 1, and further comprising:
at least one wheel configured to move the surface cleaning apparatus over the surface;
a pump operably coupled to the first liquid line; and
a motor configured to operably drive the at least one wheel.

7. The apparatus of claim 1, and further comprising:
an electrode electrically coupled to the dispenser; and
a control circuit adapted to generate an alternating electric field between the electrode and the surface, through the dispensed treated liquid, without a corresponding return electrode.

8. The cartridge of claim 1, wherein the at least one liquid-activating material comprises a zeolite.

9. The apparatus of claim 8, wherein the zeolite is selected from the group consisting of analcime, amicite, barrerite, bellbergite, bikitaite, boggsite, brewsterite, chabazite, clinoptilolite, cowlesite, dachiardite, edingtonite, epistilbite, erionite, faujasite, ferrierite, garronite, gismondine, gobbinsite, gmelinite, gonnardite, goosecreekite, harmotome, heulandite, laumontite, levyne, mazzite, merlinoite, montesommaite, mordenite, mesolite, natrolite, offretite, paranatrolite, paulingite, perlialite, phillipsite, pollucite, scolecite, stellerite, stilbite, thomsonite, tschernichite, wairakite, wellsite, willhendersonite, yugawaralite, anhydrous forms thereof, and combinations thereof.

10. The cartridge of claim 1, wherein the at least one liquid-activating material comprises a material selected from the group consisting of apophyllite, gyrolite, hsianghualite, kehoeite, lovdarite, maricopaite, okenite, pahasapaite, partheite, prehnite, roggianite, tacharanite, tiptopite, tobermorite, viseite, and combinations thereof.

11. The cartridge of claim 1, wherein the at least one liquid-activating material comprises an ion-exchange resin.

12. A cartridge for use with a surface cleaning apparatus, the cartridge comprising:
a housing that defines an interior chamber;
a first housing interface configured to provide access to the interior chamber, and further configured to be removably engaged with an upstream liquid line of the surface cleaning apparatus;
a second housing interface configured to provide access to the interior chamber, and further configured to be removably engaged with a downstream liquid line of the surface cleaning apparatus; and
media of at least one liquid-activating material retained in the interior chamber of the housing, the media of the at least one liquid-activating material being configured to modify an oxidation reduction potential of liquid flowing through the interior chamber of the cartridge.

13. The cartridge of claim 12, wherein the at least one liquid-activating material comprises a zeolite.

14. The cartridge of claim 13, wherein the zeolite is selected from the group consisting of analcime, amicite, barrerite, bellbergite, bikitaite, boggsite, brewsterite, chabazite, clinoptilolite, cowlesite, dachiardite, edingtonite, epistilbite, erionite, faujasite, ferrierite, garronite, gismondine, gobbinsite, gmelinite, gonnardite, goosecreekite, harmotome, heulandite, laumontite, levyne, mazzite, merlinoite, montesommaite, mordenite, mesolite, natrolite, offretite, paranatrolite, paulingite, perlialite, phillipsite, pollucite, scolecite, stellerite, stilbite, thomsonite, tschernichite, wairakite, wellsite, willhendersonite, yugawaralite, anhydrous forms thereof, and combinations thereof.

15. The cartridge of claim 12, wherein the at least one liquid-activating material comprises a material selected from the group consisting of apophyllite, gyrolite, hsianghualite, kehoeite, lovdarite, maricopaite, okenite, pahasapaite, partheite, prehnite, roggianite, tacharanite, tiptopite, tobermorite, viseite, and combinations thereof.

16. The cartridge of claim 12, wherein the at least one liquid-activating material comprises an ion-exchange resin.

17. The cartridge of claim 12, and further comprising:
a first media filter disposed adjacent to the first housing interface; and
a second media filter disposed adjacent to the second housing interface.

18. A method for operating a surface cleaning apparatus, the method comprising:
moving the surface cleaning apparatus relative to a surface;
introducing a liquid from a first liquid line of the surface cleaning apparatus to a container carried by the surface cleaning apparatus, the container retaining media of at least one liquid-activating material;
treating the introduced liquid with the media to modify an oxidation reduction potential of the introduced liquid to provide a treated liquid; and
dispensing the treated liquid from a dispenser of the surface cleaning apparatus located downstream from the container.

19. The method of claim 18, wherein the at least one liquid-activating material comprises a material selected from the group consisting of zeolites, ion-exchange resins, and combinations thereof.

20. The method of claim 19, wherein the container comprises a cartridge, and wherein the method further comprises:
engaging a first housing interface of the cartridge with the first liquid line such that the cartridge is located downstream from the first liquid line; and
engaging a second housing interface of the cartridge with a second liquid line of the cleaning apparatus, the second liquid line between located upstream from the dispenser.

21. The method of claim 20, and further comprising:
disengaging the first housing interface of the cartridge from the first liquid line; and
disengaging the second housing interface of the cartridge from the second liquid line.

22. The method of claim 18, wherein dispensing the treated liquid from the surface cleaning apparatus to a surface creates an electrically conductive path by the treated liquid from the surface cleaning apparatus to the surface, and wherein the method further comprises, during the step of dispensing, generating an alternating electric field from the surface cleaning apparatus to the surface, through the treated liquid along the conductive path, wherein the electric field is sufficient to destroy at least one microorganism from the surface.

23. The method of claim 18, wherein treating the introduced liquid with the media modifies the oxidation reduction potential of the introduced liquid by at least about 50 millivolts.

24. An apparatus for cleaning a surface, the apparatus comprising:
a first liquid line configured to receive a liquid;
a container coupled to the first liquid line, the container being configured to retain media of at least one liquid-activating material, the media of the at least one liquid-activating material being configured to modify an oxidation reduction potential of the received liquid to provide a treated liquid;
a second liquid line coupled to the container and configured to receive the treated liquid from the container; and
a dispenser operably coupled to the second liquid line at a location downstream from the container, the dispenser being configured to dispense the treated liquid.
